(19) United States
(12) Patent Application Publication
(10) Pub. No.: US 2005/0153918 A1
(43) Pub. Date: Jul. 14, 2005

(54) Methods and compositions relating to hnRNP A1, A1B, A2, and B1 nucleic acid molecules

(76) Inventors: Benoit Chabot, Sherbrooke (CA); Louise Bouchard, Fleurimont (CA); Pascale Labrecque, St-Elie d'Orford (CA); Caroline Patry, Sherbrooke (CA); Raymund Wellinger, Sherbrooke (CA)

Correspondence Address: CLARK & ELBING LLP, 101 FEDERAL STREET, BOSTON, MA, 02110, US

(21) Appl. No.: 10/943,441

(22) Filed: Sep. 17, 2004

Related U.S. Application Data
(63) Continuation-in-part of application No. PCT/CA03/00816, filed on May 30, 2003.
(60) Provisional application No. 60/384,309, filed on May 30, 2002.

(51) Int. Cl. A61K 48/00; C07H 21/02

(52) U.S. Cl. 514/44; 536/23.1

(57) ABSTRACT

The present invention provides methods for inducing cell death using hnRNP A1, A1^{B}, A2, and B1 nucleic acid molecules. The invention further provides therapeutic and diagnostic methods for neoplasia treatment relating to hnRNP A1, A1^{B}, A2, and B1 nucleic acid molecules.

### CROSS-REFERENCE TO RELATED APPLICATIONS

This application is a Continuation-in-part application of and claims priority to International Application No. PCT/CA03/00816, filed May 30, 2003, which was published in English under PCT Article 21(2), and which claims the benefit of U.S. provisional application No. 60/384,309, filed May 30, 2002, both of which are incorporated herein by reference in their entirety.

### BACKGROUND OF THE INVENTION

The invention features methods and compositions for treating neoplasia.

Telomeres are found at the ends of vertebrate chromosomes and are comprised of variable numbers of TTAGGG repeats in double-stranded form followed by a single-stranded overhang of G-rich repeats. The size of the overhang is estimated to be approximately 150-300 nucleotides in length and at least a portion of this extension invades the preceding double-stranded telomeric DNA to form a T-loop. The mammalian proteins TRF1 and TRF2 bind directly to double-stranded telomeric DNA and are important for telomere biogenesis. Proteins that interact specifically with the single-stranded repeats include the heterogenous nuclear ribonucleoproteins (hnRNPs) A1 and A2, as well as the recently discovered hPot1 protein.

The ribonucleoprotein enzyme telomerase directs the synthesis of telomeric repeats onto the G-rich strand, a process that counteracts the loss of sequence that occurs at each cell division. A gradual loss of telomeric sequences is thought to lead to cellular senescence. Mutagenic events resulting in mutant cells that are able to maintain stable telomeres may precede the development of neoplasia. In approximately 85% of all tumors, stabilized telomeres are thought to be a direct consequence of the reactivation of the telomerase enzyme. Distinct mechanisms involving other pathways have also been uncovered. Telomere function is absolutely essential for the growth of neoplastic cells, irrespective of their origin. Consequently, many studies aimed at reversing the neoplastic phenotype of cells have targeted the activity of proteins involved in telomere biogenesis.

For example, the expression of a catalytically inactive form of telomerase in human neoplastic cell lines was shown to promote telomere shortening, ultimately leading to growth arrest and cell death. The use of telomerase inhibitors to promote telomere shortening in neoplastic cells is also being explored. It should be noted that the longer the telomeres are when telomerase inhibitors are administered, the more divisions a neoplastic cell sustains before telomeres reach a critical length that elicits genomic instability. Meanwhile, alternative pathways for telomere maintenance may arise and bypass the requirement for telomerase function, thereby neutralizing the effect of telomerase inhibitors.

Proteins involved in the capping function of telomeres are another attractive target for therapeutic intervention. The capping function is likely to be mediated, at least in part, by proteins that recognize the single-stranded G-rich extension at the ultimate end of chromosomes. The enzyme telomerase is probably not essential for capping because stable chromosomes exist in the absence of telomerase. Strategies that interfere with the capping function of telomeres in neoplastic cells may lead to rapid growth cell arrest and cell death. The double-stranded DNA binding telomeric protein, TRF2, likely plays a role in capping, based on its function in T-loop formation and in the ability of a dominant negative version of TRF2 to promote chromosome fusions and rapid p53-dependent programmed cell death.

hnRNP Proteins

hnRNP proteins are some of the most abundant nuclear proteins in mammalian cells. There are over 20 hnRNP proteins in human cells that associate with precursor mRNAs. Many of these influence pre-mRNA processing and other aspects of mRNA metabolism and transport. The best-characterized hnRNP protein, hnRNP A1, plays a role in the control of pre-mRNA splicing. hnRNP A1 also binds with high-affinity to telomeric single-stranded DNA sequences, and can interact simultaneously with telomerase RNA in vitro. hnRNP A1 may interact simultaneously with telomeric DNA and the human telomerase RNA in vitro. Importantly, defective A1 expression in mouse erythroleukemic cells produces short telomeres whose length is increased when normal levels of hnRNP A1 are restored or when UP1, a smaller version of A1 that is defective in alternative splicing function, is expressed. Overexpressing A1 also elicits telomere elongation in human HeLa cells.

A close homolog of hnRNP A1 is the hnRNP A2 protein (A2), which shares 69% amino acid identity with hnRNP A1. Although hnRNP A2 can bind specifically to single-stranded telomeric sequence in vitro, its role in telomere biogenesis has not yet been confirmed.

For both A1 and A2, less abundant splice variants, A1 and B1, respectively, have been described. Interestingly, A1 is overexpressed in colon cancers, and the A2/B1 proteins have been used as early markers for lung cancer.

In approximately 85% of all tumors, stabilized telomeres are thought to be a direct consequence of the reactivation of the telomerase enzyme. Telomere function is absolutely essential for the growth of neoplastic cells. Given that approximately 556,500 Americans died of neoplasia in 2003, efficient methods for the treatment of neoplasia are urgently needed.

### SUMMARY OF THE INVENTION

The present invention features methods and compositions for the modulation of hnRNP A1, A1^{B}, A2, and B1 nucleic acid molecules.

We have discovered that mammalian hnRNP A1 and A2 proteins, which bind to single-stranded extensions within telomeres, are expressed at high levels in a variety of human cancers and human and mouse neoplastic cell lines. Inhibiting expression of hnRNP A1 and hnRNP A2, or any splice variants or isoforms thereof (e.g., isoforms A1^{B} and B1, respectively), using nucleic acid molecules such as small interfering RNAs or antisense nucleobase oligomers promotes rapid apoptotic cell death, which is specifical to neoplastic cells. Since A1B is an alternatively spliced isoform of A1 and has several identical exons, a preferred antisense nucleobase oligomer or siRNA molecule will target the shared exons to downregulate the expression of both the A1 and A1B genes. Similarly, since B1 is an alternatively spliced isoform of A2 and has several identical exons, a preferred antisense nucleobase oligomer or siRNA molecule will target the shared exons to downregulate the expression of both the A2 and B1 genes. The terms “A1/A1B” or “A2/B1” are used throughout the specification to refer to the nucleic acid sequences shared by both isoforms but it will be understood that the invention also features antisense or siRNA molecules that target the unique exons of each gene, thereby downregulating the expression of only one of the isoforms.

In one aspect, the invention provides a method of inducing cell death in a cell by inhibiting the expression of hnRNP A1 or A1^{B}, preferably both, and hnRNP A2 or B1, preferably both, nucleic acid molecules or polypeptides. In one embodiment, the method involves administering to the cell (i) a first nucleic acid molecule having at least one strand that is at least 80%, preferably 85%, 90%, 95%, 99%, or 100% complementary to at least a portion of the sequence of hnRNP A1 or hnRNP A1^{B}, or splice variants or isoforms thereof, and (ii) a second nucleic acid molecule having at least one strand that is at least 80%, preferably 85%, 90%, 95%, 99%, or 100% complementary to at least a portion of the sequence of hnRNP A2 or hnRNP B1, or splice variants or isoforms thereof, where the first and second nucleic acid molecules are administered in an amount sufficient to reduce or inhibit the expression of endogenous hnRNP A1 or A1^{B}, preferably both, and hnRNP A2 or B1, preferably both, nucleic acid molecules or proteins. In preferred embodiments, the first and second nucleic acid molecules are double stranded (ds) RNA, siRNA, shRNA, or antisense nucleic acid molecules, or any combination thereof. In a preferred embodiment, the administered first and second nucleic acid molecules are stably expressed in the cell. In another preferred embodiment, the cell is a neoplastic cell. In another preferred embodiment, the neoplastic cell is a mammalian cell (e.g., a human cell or a mouse cell). In another preferred embodiment, the human or mouse cell is in vivo. In another preferred embodiment, the cell death is caused by telomere uncapping. In another preferred embodiment, the method is sufficient to induce apoptosis in a neoplastic cell, but not in a normal cell.

In additional preferred embodiments, the first nucleic acid molecule is an siRNA molecule having 100% nucleic acid identity to at least 18, preferably 19, 20, 21, 22, 23, 24, 25, 35, 45, 50 or more consecutive nucleotides of SEQ ID NOs: 27, 31, or 32, and nucleic acid molecule (ii) is an siRNA molecule having 100% nucleic acid identity to at least 18, preferably 19, 20, 21, 22, 23, 24, 25, 35, 45, 50 or more consecutive nucleotides of SEQ ID NOs: 28 or 33.

In additional preferred embodiments, the first nucleic acid molecule is an antisense nucleobase oligomer complementary to at least 10, preferably 20, 30, 40, 50, 60, 70, 80, 90, 100, or more consecutive nucleotides of SEQ ID NOs: 27, 31, or 32, and nucleic acid molecule (ii) is an antisense nucleobase oligomer complementary to at least 10, preferably 20, 30, 40, 50, 60, 70, 80, 90, 100 or more consecutive nucleotides of SEQ ID NOs: 28 or 33.

In another aspect the invention provides a method of treating a subject having a neoplasm. This method involves administering to the cell (i) a first nucleic acid molecule having at least one strand that is at least 80%, preferably 85%, 90%, 95%, 99%, or 100% complementary to at least a portion of the sequence of hnRNP A1 or hnRNP A1^{B}, or splice variants or isoforms thereof, and (ii) a second nucleic acid molecule having at least one strand that is at least 80%, preferably 85%, 90%, 95%, 99%, or 100% complementary to at least a portion of the sequence of hnRNP A2 or hnRNP B1, or splice variants or isoforms thereof, where the nucleic acid molecules are administered in an amount and for a time sufficient to reduce or inhibit the expression of endogenous hnRNP A1 or A1^{B} and hnRNP A2 or B1 nucleic acid molecules or proteins. In preferred embodiments, the nucleic acid molecules (i) and (ii) are double stranded (ds) RNA, siRNA, shRNA, or antisense nucleic acid molecules, or any combination thereof. In a preferred embodiment, the administered nucleic acid molecules (i) and (ii) are stably expressed in the cell. In another preferred embodiment, the cell is a neoplastic cell. In another preferred embodiment, the neoplastic cell is a mammalian cell (e.g., a human cell or a mouse cell). In another preferred embodiment, the human or mouse cell is in vivo. In another preferred embodiment, the cell death is caused by telomere uncapping. In another preferred embodiment, the method is sufficient to induce apoptosis in a neoplastic cell, but not in a normal cell. In other embodiments, the subject has bladder, blood, bone, brain, breast, cartilage, colon kidney, liver, lung, lymph node, nervous tissue, ovary, pancreatic, prostate cancer, skeletal muscle, skin, spinal cord, spleen, stomach, testes, thymus, thyroid, trachea, urogenital tract, ureter, urethra, uterus, or vaginal cancer. In another preferred embodiment, the method is administered in combination with any standard cancer therapy (e.g., chemotherapy, small molecule therapy, antibody therapy, radiation therapy, and surgery). Therapeutically effective concentrations of the nucleic acid molecules are determined by alterations in the concentration or activity of the DNA, RNA or gene product of A1 or A1^{B} and A2 or B1, tumor regression, or a reduction of the pathology or symptoms associated with the neoplasm.

In a related aspect, the invention provides a method of decreasing the length of single-stranded telomere extensions of chromosomes in a cell, the method comprising administering to a cell (i) a first nucleic acid molecule having at least one strand that is at least 80%, preferably 85%, 90%, 95%, 99%, or 100% complementary to at least a portion of the sequence of hnRNP A1 or hnRNP A1^{B}, preferably both, and (ii) a second nucleic acid molecule having at least one strand that is at least 80%, preferably 85%, 90%, 95%, 99%, or 100% complementary to at least a portion of the sequence of hnRNP A2 or hnRNP B1, preferably both, where the first and second nucleic acid molecules are administered in an amount sufficient to reduce the expression of endogenous hnRNP A1 or A1^{B} and hnRNP A2 or B1 nucleic acid molecules or proteins. In one embodiment, the cell death is the result of increased telomere or chromosome fusion, or telomere uncapping.

In any of the methods of the invention, the nucleic acid molecule can be a dsRNA, siRNA, shRNA, or antisense nucleic acid molecule. In preferred embodiments the nucleic acid molecule is an siRNA molecule with 100% nucleic acid sequence identity to at least 18, 19, 20, 21, 22, 23, 24, 25, 35, 45, 50 or more consecutive nucleotides of any one of SEQ ID NOs: 27, 28, and 31-33. In additional preferred embodiments, the nucleic acid molecule is an antisense molecule that is at least 80%, preferably 85%, 90%, 95%, 99%, or 100% complementary to at least 10, 20, 30, 40, 50, 60, 70, 80, 90, or 100 consecutive nucleotides of any one of SEQ ID NOs: 27, 28, and 31-33.

1. In another aspect, the invention features a purified siRNA molecule having at least one strand that is at least 80%, preferably 85%, 90%, 95%, 99%, or 100% complementary to at least a portion of any one of the sequences set forth in SEQ ID NOs: 27, 31, and 32, where the siRNA molecule can reduce the level of a nucleic acid having at least one of SE ID NOs 27, 31, and 32 in a cell in which the nucleic acid is expressed. In preferred embodiments, the siRNA molecule is 100% complementary to at least 18, preferably 19, 20, 21, 22, 23, 24, 25, 35, 45, 50 or more consecutive nucleotides of any one of the sequences set forth in SEQ ID NOs: 27, 31, and 32. In additional preferred embodiments, the siRNA has at least one strand that is 100% complementary to at least 18 consecutive nucleotides of both SEQ ID NOs: 27 and 32 or both SEQ ID NOs: 31 and 32. Desirably, the siRNA has at least one strand that is 100% complementary to at least a portion of one of the following sequences: nucleotides 1 to 865 of SEQ ID NO: 27 and nucleotides 857 to 1769 of SEQ ID NO: 27.

In another aspect, the invention features a purified nucleic acid molecule having at least one strand that is at least 80%, preferably 85%, 90%, 95%, 99%, or 100% complementary to at least a portion of any of one of the sequences set forth in SEQ ID NOs: 28 and 33, and where the nucleic acid molecule can reduce the level of a nucleic acid having the sequence set forth in SEQ ID NOs: 28 or 33 in a cell. In preferred embodiments, the nucleic acid molecule is an siRNA molecule. Desirably, the siRNA molecule is 100% complementary to at least 18, preferably 19, 20, 21, 22, 23, 24, 25, 35, 45, 50 or more consecutive nucleotides of any one the sequence set forth in SEQ ID NOs: 28 and 33. In a preferred embodiment, the siRNA has at least one strand that is 100% complementary to at least 18 consecutive nucleotides of both SEQ ID NOs: 28 and 33. Desirably, the siRNA has at least one strand that is 100% complementary to at least 18 consecutive nucleotides of the following sequences: nucleotides 1 to 176 of SEQ ID NO: 28 and nucleotides 177 to 1714 of SEQ ID NO: 28.

In other preferred embodiments, the nucleic acid molecule is an antisense nucleobase oligomer molecule. Desirably, the antisense nucleobase oligomer is 80%, 85%, 90%, 95%, or 100% complementary to at least 10, preferably 20, 30, 40, 50, 60, 70, 80, 90, 100, or more consecutive nucleotides of any one the sequence set forth in SEQ ID NOs: 28 and 33. In a preferred embodiment the antisense nucleobase oligomer has at least one strand that is 100% complementary to at least 10 consecutive nucleotides of the following sequences: nucleotides 1 to 176 of SEQ ID NO: 28 and nucleotides 177 to 1714 of SEQ ID NO: 28.

In another aspect, the invention features a pharmaceutical composition comprising (i) a first nucleic acid molecule having at least one strand that is at least 80%, preferably 85%, 90%, 95%, 99%, or 100% complementary to at least a portion of a nucleic acid sequence of SEQ ID NOs: 27, 31, or 32, and (ii) a second nucleic acid molecule comprising at least one strand that is at least 80%, preferably 85%, 90%, 95%, 99%, or 100% complementary to at least a portion of a nucleic acid sequence of SEQ ID NOs: 28 or 33, where the first nucleic acid molecule reduces the level expression of a nucleic acid having at least one of SEQ ID NOs: 27, 31, or 32, and the second nucleic acid molecule reduces the level of a nucleic acid having at least one of SEQ ID NOs: 28 or 33. In preferred embodiments, the first and second nucleic acid molecules are dsRNA, siRNA, shRNA, or antisense nucleobase oligomer. In other preferred embodiments, the first siRNA molecule is 100% complementary to at least 18, 19, 20, 21, 22, 23, 24, 25, 35, 45, 50 or more consecutive nucleotides of SEQ ID NOs: 27, 31, or 32, and the second siRNA is 100% complementary to at least 18, 19, 20, 21, 22, 23, 24, 25, 35, 45, 50 or more consecutive nucleotides of SEQ ID NOs: 28 or 33.

In a preferred embodiment, the first nucleic acid molecule is selected from SEQ ID NOs: 1-16 and 29-30, and the second nucleic acid molecule is selected from SEQ ID NOs: 17-26. In another preferred embodiment, the first siRNA molecule is 100% complementary to at least 18, 19, 20, 21, 22, 23, 24, 25, 35, 45, 50 or more consecutive nucleotides of both SEQ ID NOs: 27 and 32 or both 31 and 32, and the second siRNA is 100% complementary to at least 18, 19, 20, 21, 22, 23, 24, 25, 35, 45, 50 or more consecutive nucleotides of both SEQ ID NOs: 28 and 33. Desirably, the first siRNA is 100% complementary to 18 to 25 consecutive nucleotides of nt 1 to 865 of SEQ ID NO: 27 or nt 857 to 1769 of SEQ ID NO: 27, and the second siRNA is 100% complementary to 18 to 25 consecutive nucleotides of nt 1 to 176 of SEQ ID NO: 28 or 177 to 1714 of SEQ ID NO: 28.

In another preferred embodiment, the first and second nucleic acid molecules are antisense nucleobase oligomers. In preferred embodiments, the antisense nucleobase oligomer is complementary to at least 10, 20, 30, 40, 50, 60, 70, 80, 90, or 100 consecutive nucleotides of SEQ ID NOs: 27, 31, or 32, and the second si/rna is 100% complementary to at least 10, 20, 30, 40, 50, 60, 70, 80, 90, or 100 consecutive nucleotides of SEQ ID NOs: 28 or 33.

In another preferred embodiment, the first antisense nucleobase oligomer is 100% complementary to at least 10, 20, 30, 40, 50, 60, 70, 80, 90, or 100 consecutive nucleotides of both SEQ ID NOs: 27 and 32 or both 31 and 32, and the second antisense nucleobase oligomer is 100% complementary to at least 10, 20, 30, 40, 50, 60, 70, 80, 90, or 100 consecutive nucleotides of both SEQ ID NOs: 28 and 33. Desirably, the first antisense nucleobase oligomer is 100% complementary to 10, 20, 30, 40, 50, 60, 70, 80, 90, or 100 consecutive nucleotides of nt 1 to 865 of SEQ ID NO: 27 or nt 857 to 1769 of SEQ ID NO: 27, and the second antisense nucleobase oligomer is 100% complementary to 10, 20, 30, 40, 50, 60, 70, 80, 90, or 100 consecutive nucleotides of nt 1 to 176 of SEQ ID NO: 28 or 177 to 1714 of SEQ ID NO: 28.

In a related aspect, the invention provides a pharmaceutical composition containing at least one pair of double stranded nucleic acid molecules selected from the following group of pairs of double stranded nucleic acid molecules: SEQ ID NOs: 1 and 2, 3 and 4, 5 and 6, 7 and 8, 9 and 10, 11 and 12, 13 and 14, 15 and 16, 17 and 18, 19 and 20, 21 and 22, 23 and 24, 25 and 26, and 29 and 30, in a pharmaceutically acceptable carrier. The pharmaceutical composition can also include at least two of the above pairs of double stranded nucleic acid molecules. In a preferred embodiment, the first pair is selected from the following pairs of nucleic acid molecules: SEQ ID NOs: 1 and 2; 3 and 4; 5 and 6; 7 and 8; 9 and 10; 11 and 12; 13 and 14; 15 and 16; and 29 and 30, and the second pair is selected from the following pairs of nucleic acid molecules: SEQ ID NOs: 17 and 18; 19 and 20; 21 and 22; 23 and 24; 25 and 26. In preferred embodiments any of the pairs of nucleic acid molecules are joined by a base linker region.

In another aspect, the invention features a composition comprising at least one antisense nucleobase oligomer selected from the group consisting of any one or more of the following SEQ ID NOs: 18, 20, 22, 24, and 26.

Any of the above compositions can also include a pharmaceutically acceptable carrier.

In another aspect, the invention features a kit for the treatment of a neoplasia in a patient comprising any of the nucleic acid molecules of the invention. In a preferred embodiment, the kit includes at least one pair of double stranded nucleic acid molecules selected from the following group of pairs of double stranded nucleic acid molecules: SEQ ID NOs 1 and 2, 3 and 4, 5 and 6, 7 and 8, 9 and 10, 11 and 12, 13 and 14, 15 and 16, 17 and 18, 19 and 20, 21 and 22, 23 and 24, 25 and 26, and 29 and 30. The kit can also include at least two of the above pairs of double stranded nucleic acid molecules. In a preferred embodiment, the first pair is selected from the following pairs of nucleic acid molecules: SEQ ID NOs: 1 and 2; 3 and 4; 5 and 6; 7 and 8; 9 and 10; 11 and 12; 13 and 14; 15 and 16; and 29 and 30, and the second pair is selected from the following pairs of nucleic acid molecules: SEQ ID NOs: 17 and 18; 19 and 20; 21 and 22; 23 and 24; 25 and 26.

In a preferred embodiment, the kit includes (i) a first nucleic acid molecule having at one strand that is at least 95% complementary to at least a portion of any one of the sequences set forth in SEQ ID NOs: 27, 31, and 32, and (ii) a second nucleic acid molecule having at least one strand that is at least 95% complementary to at least a portion of any one of the sequences set forth in SEQ ID NOs: 28 and 33, wherein the first nucleic acid molecule reduces the level of at least one of the nucleic acid sequences set forth in SEQ ID NOs: 27, 31, and 32 in a cell, and the second nucleic acid molecule reduces the level of at least one of said nucleic acid sequences set forth in SEQ ID NOs: 28 and 33 in a cell. In preferred embodiments, the first and second nucleic acid molecules can also induce apoptosis in the cell.

In a related aspect, the invention features a kit for the treatment of a neoplasia in a patient comprising at least one antisense nucleobase oligomer selected from the group consisting of SEQ ID NO: 18, 20, 22, 24, and 26.

In another aspect, the invention features a method of diagnosing a patient as having, or having a propensity to develop, a neoplasia, the method comprising determining the level of expression of an hnRNPA1 or A1^{B} or hnRNPA2 or B1 nucleic acid molecule or polypeptide in a patient sample, where an increased level of expression relative to the level of expression in a control sample, indicates that the patient has or has a propensity to develop a neoplasia. In one embodiment, the method involves determining the level of expression of the hnRNPA1 or A1^{B} nucleic acid molecule. In another embodiment, the method involves determining the level of expression of the hnRNPA2 or B1 nucleic acid molecule. In a preferred embodiment, the method involves determining the level of expression of hnRNPA1 or A1^{B} and hnRNPA2 or B1 nucleic acid molecules. In another preferred embodiment, the method involves determining the level of expression of the hnRNP A1 or A1^{B} and hnRNPA2 or B1 polypeptides. In one embodiment, the level of polypeptide expression is determined in an immunological assay.

In another aspect, the invention features a diagnostic kit for the diagnosis of a neoplasia in a patient comprising a nucleic acid sequence, or fragment thereof, of at least one of an hnRNPA1 or A1^{B} and at least one of hnRNPA2 or B1 nucleic acid molecule. In a preferred embodiment, the diagnostic kit includes (i) a first nucleic acid molecule having at one strand that is at least 95% complementary to at least a portion of any one of the sequences set forth in SEQ ID NOs: 27, 31, and 32, and (ii) a second nucleic acid molecule having at least one strand that is at least 95% complementary to at least a portion of any one of the sequences set forth in SEQ ID NOs: 28 and 33

In another aspect, the invention features a method of identifying a candidate compound that ameliorates a neoplasia, the method comprising contacting a cell that expresses hnRNPA1 or A1^{B}, or both, and an hnRNPA2 or B1, or both, nucleic acid molecule with a candidate compound, and comparing the level of expression of the nucleic acid molecule in the cell contacted by the candidate compound with the level of expression in a control cell not contacted by the candidate compound, where a decrease in expression of the hnRNP A1, A1^{B}, hnRNP A2, or B1 nucleic acid molecules, or any combination thereof, identifies the candidate compound as a candidate compound that ameliorates a neoplasia. In one embodiment, the decrease in expression is a decrease in transcription. In another embodiment, the decrease in expression is a decrease in translation.

In another aspect, the invention features a method of identifying a candidate compound that ameliorates a neoplasia, the method comprising contacting a cell that expresses an hnRNP A1, A1^{B}, hnRNP A2 or B1 polypeptide with a candidate compound, and comparing the level of expression of the polypeptide in the cell contacted by the candidate compound with the level of polypeptide expression in a control cell not contacted by the candidate compound, where a decrease in the expression of the hnRNP A1, A1^{B}, hnRNP A2 or B1 polypeptide, or any combination thereof, identifies the candidate compound as a candidate compound that ameliorates a neoplasia. In one embodiment, the decrease in expression is assayed using an immunological assay, an enzymatic assay, or a radioimmunoassay.

In another aspect, the invention features a method of inducing cell death in a cell by inhibiting the expression of an hnRNP A2 or B1, or splice variants or isoforms thereof, nucleic acid molecule or polypeptide. In a preferred embodiment, the method involves administering to the cell a nucleic acid molecule having at least one strand that is at least 80%, preferably 85%, 90%, 95%, 99%, or 100% complementary to at least a portion of the sequence of hnRNP A2 or B1, where the nucleic acid molecule is administered in an amount sufficient to reduce the expression of an hnRNP A2 or B1 nucleic acid molecule or protein. In a preferred embodiment, the cell death is caused by telomere uncapping. In another preferred embodiment, the administered nucleic acid molecules are double stranded nucleic acid molecules, siRNA or antisense nucleic acid molecules. In additional preferred embodiments, the nucleic acid is stably expressed in the cell (e.g., a neoplastic human or mouse cell). In additional preferred embodiment, the cell is in vivo.

In another aspect, the invention features a vector comprising any of the nucleic acid molecules of the invention. In preferred embodiments, the nucleic acid molecule is positioned for expression, where the nucleic acid molecule encodes a nucleic acid molecule having at least one strand that is at least 80%, preferably 85%, 90%, 95%, 99%, or 100% complementary to at least a portion of the sequence of one of SEQ ID NOs: 27, 31, or 32. In preferred embodiments, the nucleic acid molecule is a double stranded nucleic acid molecule, preferably an siRNA molecule. In additional preferred embodiments the nucleic acid molecule is an siRNA molecule that is 100% complementary to at least 18, 19, 20, 21, 22, 23, 24, 25, 35, 45, 50 or more consecutive nucleotides of SEQ ID NOs: 27, 31, or 32.

In another aspect, the invention features a vector comprising a nucleic acid molecule positioned for expression, where the nucleic acid molecule encodes a nucleic acid molecule having at least one strand that is at least 80%, preferably 85%, 90%, 95%, 99%, or 100% complementary to at least a portion of the sequence of SEQ ID NOs: 28 or 33, and reduces or inhibits the expression of SEQ ID NOs: 28 or 33. In preferred embodiments, the nucleic acid molecule is a double stranded nucleic acid molecule, an siRNA molecule or an antisense molecule. In additional preferred embodiments the nucleic acid molecule is an siRNA molecule that is 100% complementary to at least 18, 19, 20, 21, 22, 23, 24, 25, 35, 45, 50 or more consecutive nucleotides of SEQ ID NOs: 28 or 33. In yet additional preferred embodiments, the nucleic acid molecule is an antisense nucleobase oligomer molecule that is 100% complementary to at least 10, 20, 30, 40, 50, 60, 70, 80, 90, or 100 nucleotides of SEQ ID NOs: 28 or 33.

In another aspect, the invention features a vector comprising a nucleic acid molecule positioned for expression, where the first nucleic acid molecule encodes (i) a first nucleic acid molecule having at least one strand that is at least 80%, preferably 85%, 90%, 95%, 99%, or 100% complementary to at least a portion of the sequence of any one of SEQ ID NOs: 27, 31, and 32, and (ii) a second nucleic acid molecule having at least one strand that is c, where the first nucleic acid molecule reduces the level of at least one of the nucleic acid sequence of SEQ ID NOs: 27, 31, or 32 in a cell, and the second nucleic acid molecule reduces the level of at least one of the nucleic acid sequence of SEQ ID NOs: 28 or 33 in a cell. In preferred embodiments, the first or second nucleic acid molecule is an siRNA molecule, where the first siRNA is 100% complementary to at least 18, 19, 20, 21, 22, 23, 24, 25, 35, 45, 50 or more consecutive nucleotides of SEQ ID NOs: 27, 31, or 32, and the siRNA of (ii) is 100% complementary to at least 18, 19, 20, 21, 22, 23, 24, 25, 35, 45, 50 or more consecutive nucleotides of SEQ ID NOs: 28 or 33. In preferred embodiments, the first nucleic acid molecule is at least 80%, preferably 85%, 90%, 95%, 99%, or 100% complementary to at least a portion of the sequence of both SEQ ID NOs: 27 and 32 or 31 and 32, and the second nucleic acid molecule is at least 80%, preferably 85%, 90%, 95%, 99%, or 100% complementary to both SEQ ID NOs: 28 and 33. In another preferred embodiment, the first or second nucleic acid molecule or both is an antisense nucleobase oligomer. In preferred embodiments the first antisense nucleic acid molecule is complementary to at least 10, 20, 30, 40, 50, 60, 70, 80, 90, or 100 consecutive nucleotides of SEQ ID NOs: 27, 31, or 32, and the second antisense nucleic acid molecule is complementary to at least 10, 20, 30, 40, 50, 60, 70, 80, 90, or 100 nucleotides of SEQ ID NOs: 28 or 33. The first and second molecule and either be siRNA or an antisense nucleobase oligomer or any combination thereof.

In another aspect, the invention features a method of using the nucleic acid molecules of the previous aspects to induce apoptosis in a cell. In preferred embodiments, the cell is a neoplastic cell. In other preferred embodiment, the neoplastic cell is in a human or a mouse.

In another aspect, the invention features a method of using a nucleic acid molecule of any previous aspect to treat a subject having a neoplasm. In a preferred embodiment, the subject is a mammal, preferably a human.

In another aspect, the invention features a method of using the nucleic acid molecule of any of the previous aspects to decrease the length of single-stranded telomere extensions of chromosomes in a cell.

In various preferred embodiments of any of the aspects of the invention, the nucleic acid molecules are dsRNAs, siRNAs, shRNAs, or antisense nucleic acid molecules. In preferred embodiments, the methods of the invention also include the use of any combination of nucleic acid molecules of the invention. In other preferred embodiments of any of the above aspects, the nucleic acid molecules are stably expressed in a cell (e.g., a mammalian, human, or neoplastic cell). In preferred embodiments of any of the above aspects, the human cell is in vivo. In other embodiments of the above aspects, cell death is caused by telomere uncapping.

In other preferred embodiments of any of the above aspects, the nucleic acid is an siRNA that is 85%, 90%, 95%, or 100% complementary to at least 15, 16, 17, 18, 19, 20, 21, 22, 23, 24, 25, 34, 45, 50 or more consecutive nucleotides of SEQ ID NOs: 27, 28, or 31-33. Preferred siRNA molecules include any one of SEQ ID NOs: 1, 2, 3, 4, 5, 6, 7, 8, 9, 10, 11, 12, 13, 14, 15, 16, 17, 18, 19, 20, 21, 22, 23, 24, 25, and 26, 29, and 30. In other embodiments of any of the above aspects, the nucleic acid molecule is an antisense nucleic acid molecule that is 85%, 90%, 95%, or 100% complementary to at least 5, 10, 20, 30, 40, 50, 60, 70, 80, 90, 100, 200, 300, 400, or 500 consecutive nucleotides of SEQ ID NOs: 27, 28, or 31-33.

By “antisense nucleobase oligomer” is meant a nucleic acid sequence, regardless of length, that is complementary to the coding strand, or mRNA, of an hnRNP A1, hnRNP A1^{B}, hnRNP A2, or hnRNP A2/B1 gene. Preferably, the antisense nucleobase oligomer is capable of reducing or inhibiting the expression of at least one of the following: hnRNP A1, hnRNP A1^{B}, hnRNP A2, or hnRNP A2/B1 in a cell by at least 10%, 20%, 30%, 40%, or more preferably by at least 50%, 60%, 70%, or 75%, or even by as much as 80%, 90%, or 95% relative to an untreated control cell. By a “nucleobase oligomer” is meant a compound that includes a chain of at least eight nucleobases, preferably at least twelve, and most preferably at least sixteen bases, joined together by linkage groups. Included in this definition are natural and non-natural nucleic acid molecules, both modified and unmodified, as well as oligonucleotide mimetics such as Protein Nucleic Acids, locked nucleic acids, and arabinonucleic acids. Examples of numerous nucleobases and linkage groups that may be used in the nucleobase oligomers of the invention can be found in U.S. Patent Publication Nos. 20030114412 (see, for example, paragraphs 27-45 of the publication) and 20030114407 (see for example paragraphs 35-52 of the publication). The nucleobase oligomer can also be targeted to the translational start and stop sites. An antisense nucleobase oligomer may also contain at least 10, 15, 20, 25, 30, 40, 60, 85, 120, or more consecutive nucleotides that are complementary to hnRNP A1, hnRNP A1^{B}, hnRNP A2, or hnRNP A2/B1 mRNA or DNA, and may be as long as a full-length hnRNP A1, hnRNP A1^{B}, hnRNP A2, or hnRNP A2/B1 gene or mRNA. Preferably an antisense nucleobase oligomer includes from about 8 to 30 nucleotides.

By “candidate compound” is meant any nucleic acid molecule, polypeptide, or other small molecule, that is assayed for its ability to alter gene or protein expression levels, or the biological activity of a gene or protein by employing one of the assay methods described herein. Candidate compounds include, for example, peptides, polypeptides, synthesized organic molecules, naturally occurring organic molecules, nucleic acid molecules, and components thereof.

By “cell death” is meant apoptosis. Apoptosis is a highly regulated form of cell death characterized by one or more of the following features: cell shrinkage, membrane blebbing, internucleosomal DNA cleavage, and chromatin condensation culminating in cell fragmentation.

By “complementary” or “complementarity” is meant polynucleotides (i.e., a sequence of nucleotides) related by the nucleobase-pairing rules. For example, for the sequence “A-G-T,” is complementary to the sequence “T-C-A.” Complementarity may be “partial,” in which only some of the nucleic acid bases are matched according to the base pairing rules. In preferred embodiments, a partially or substantially complementary nucleic acid molecule has at least 80%, preferably 85%, 90%, 95%, or 99% of its bases matched to the bases in the comparison molecule according to the base pairing rules. Or, there may be “complete” or “total” complementarity between the nucleic acids. Sequence complementarity is measured using the same methods as described for measuring sequence identity, below. In a preferred embodiment, sequence complementarity is measured for a given number of consecutive residues and excludes additional residues such as overhang residues.

By “hybridize” is meant pair to form a double-stranded molecule between complementary polynucleotide sequences, or portions thereof, under various conditions of stringency. (See, e.g., Wahl, G. M. and S. L. Berger (1987) Methods Enzymol. 152: 399; Kimmel, A. R. (1987) Methods Enzymol. 152: 507) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196: 180, 1977); Grunstein and Hogness (Proc. Natl. Acad. Sci., USA 72: 3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “differentially expressed” is meant a difference in the expression level of a nucleic acid molecule or polypeptide. This difference may be either an increase or a decrease of at least 10%, 20%, 30%, 40%, 50%, 60%, 70%, 80%, 90% or 95% in expression, relative to a reference or to control expression.

By “effective amount” is meant an amount sufficient to arrest, ameliorate, or inhibit the continued proliferation, growth, or metastasis (e.g., invasion, or migration) of a neoplasia.

By “neoplastic cell” is meant a cell multiplying or growing in an abnormal, uncontrolled manner. A neoplastic cell grows in conditions that would inhibit the proliferation of a normal cell.

By “decreasing telomere length” is meant reducing the overall number of terminal repeats (TTAGGG) found in the telomere. In general the overall length of a shortened telomere, as used herein, includes telomeres from 3 kB to 12 kB, more preferably 5 kB to 10 kB, most preferably 5 kB to 8 kB. In general the rate of telomere shortening will range from 20 to 200 nucleotides per population doubling, with a more preferable rate of 30 to 150 nucleotides per population doubling, and a most preferable rate of 40 to 100 nucleotides per population doubling.

By “decreasing single-stranded G-rich strand telomeric overhang” is meant reducing the number of single-stranded TTAGGG repeats found at the very 3′-end of chromosomes. The preferred length of telomere 3′ single stranded G-rich overhang is 50 to 400 nucleotides and more preferably 125 to 275 nucleotides (Cimino-Reale et al., Nucl. Acids Res. 27: e35, 2001; Wright et al., Genes and Dev. 11: 2801-2809, 1997).

By “dsRNA” is meant a ribonucleic acid molecule having both a sense and an anti-sense strand.

By “hnRNP A1 nucleic acid molecule” or “A1 nucleic acid molecule” is meant a nucleic acid molecule (e.g., DNA, cDNA, genomic, mRNA, RNA, dsRNA, antisense RNA, shRNA) substantially identical to GenBank accession number NM_{—}002136 (SEQ ID NO:27) or NM_{—}010447 (SEQ ID NO: 31), including any splice variants or isoforms thereof.

By “hnRNP A1^{B}” or “A1^{B}” is meant a nucleic acid molecule (e.g., DNA, cDNA, genomic, mRNA, RNA, dsRNA, antisense RNA, shRNA) substantially identical to GenBank accession number NM_{—}031157 (SEQ ID NO: 32), including any splice variants or isoforms thereof. “hnRNP A1^{B}” is an alternatively spliced isoform of hnRNP A1 that utilizes exon 7B resulting in an mRNA product with an additional 156 nucleotides as compared to hnRNP A1.

By “hnRNPA2 nucleic acid molecule” or “A2 nucleic acid molecule” is meant a nucleic acid molecule (e.g., DNA, cDNA, genomic, mRNA, RNA, dsRNA, antisense RNA, shRNA) that is substantially identical to GenBank accession number NM_{—}002137 (SEQ ID NO: 28), including any splice variants or isoforms thereof.

By “hnRNP A2/B1” or “A2/B1” is meant a nucleic acid molecule (e.g., DNA, cDNA, genomic, mRNA, RNA, dsRNA, antisense RNA, shRNA) substantially identical to GenBank accession number NM_{—}031243 (SEQ ID NO: 33), including any splice variants or isoforms thereof “hnRNP B1” is an alternatively spliced isoform of hnRNP A2 that includes exon 2 and has an additional 36 nucleotides at the beginning of the coding region and, as a result, has a different amino-terminus than hnRNP A2. hnRNP B0 is an alternatively spliced isoform of hnRNP A2 that lacks exon 9.

By “hnRNP A1 polypeptide” or “A1 polypeptide” is meant a polypeptide encoded by an hnRNP A1 nucleic acid sequence. By “hnNPA1^{B}” polypeptide is meant a polypeptide encoded by an hnRNP A1^{B} nucleic acid sequence or a polypeptide substantially identical to GenBank accession number NP_{—}112420 (SEQ ID NO.: 34). Such polypeptides belong to the A/B subfamily of ubiquitously expressed hnRNPs. The biological activities of hnRNP A1 polypeptide include binding to RNA, and contributing to the regulation of pre-mRNA processing, mRNA metabolism, mRNA transport, and telomere biogenesis.

By “hnRNP A2 polypeptide” or “A2 polypeptide” is meant a protein encoded by an hnRNP A2 nucleic acid molecule. By “hnRNP A2/B1” polypeptide” is meant a polypeptide encoded by an hnRNP A2/B1 nucleic acid sequence of a polypeptide substantially identical to GenBank accession number NP_{—}112533 (SEQ ID NO: 35). Such polypeptides belong to the A/B subfamily of ubiquitously expressed hnRNPs. The biological activities of hnRNP A1 polypeptide include binding to RNA, and contributing to the regulation of pre-mRNA processing, mRNA metabolism, mRNA transport, and telomere biogenesis.

By “neoplasm” is meant an abnormal tissue that grows by a rapid, uncontrolled cellular proliferation and continues to grow after the stimuli that initiated the new growth cease. Neoplasms show partial or complete lack of structural organization and functional coordination with the normal tissue, and usually form a distinct mass of tissue, which may be either benign or malignant.

By “neoplasia” is meant a disease characterized by the pathological proliferation of a cell or tissue. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

By “nucleic acid molecule” is meant any chain of nucleotides or nucleic acid mimetics. Included in this definition are natural and non-natural oligonucleotides, both modified and unmodified.

By “pharmaceutically acceptable carrier” is meant a carrier that is physiologically acceptable to the treated mammal while retaining the therapeutic properties of the compound with which it is administered. One exemplary pharmaceutically acceptable carrier substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa. The term “pharmaceutically acceptable salts” refers to salts that retain the desired biological activity of the parent compound and do not impart undesired toxicological effects thereto. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., J. Pharma Sci., 66: 1-19, 1977). The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides and other nucleobase oligomers, suitable pharmaceutically acceptable salts include (i) salts formed with cations such as sodium, potassium, ammonium, magnesium, calcium, polyamines such as spermine and spermidine, etc.; (ii) acid addition salts formed with inorganic acids, for example hydrochloric acid, hydrobromic acid, sulfuric acid, phosphoric acid, nitric acid and the like; (iii) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (iv) salts formed from elemental anions such as chlorine, bromine, and iodine.

By “reduce or inhibit” is meant the ability to cause an overall decrease by 20%, 30%, or 40%, more preferably by 50%, 60%, or 70%, most preferably by 80%, 90%, or even 100% in the level of protein or nucleic acid as compared to samples not treated with the nucleic acid molecules of the invention. This reduction or inhibition of RNA or protein expression can occur through targeted mRNA cleavage or degradation. Assays for protein expression or nucleic acid expression are known in the art and include, for example, ELISA and western blot analysis for protein expression, Southern blotting or PCR for DNA analysis, and northern blotting, PCR, or RNase protection assays for RNA.

By “RNA interference (RNAi)” is meant the administration of a nucleic acid molecule (e.g., antisense, shRNA, siRNA, dsRNA), regardless of length, that inhibits the expression of an hnRNP A1, hnRNP A1^{B}, hnRNP A2, or hnRNP A2/B1 gene, and splice variants or isoforms thereof, or any combination thereof. Typically, the administered nucleic acid molecule contains one strand that is complementary to the coding strand of an mRNA of hnRNP A1, hnRNP A1^{B}, hnRNP A2, or hnRNP A2/B1 gene. RNAi is a form of post-transcriptional gene silencing initiated by the introduction of double-stranded RNA (dsRNA) or antisense RNA. Preferably, RNAi is capable of decreasing the expression of at least one, more preferably two, three, or all four of hnRNP A1, hnRNP A1^{B}. hnRNP A2, or hnRNP A2/B1 in a cell by at least 10%, 20%, 30%, or 40%, more preferably by at least 50%, 60%, or 70%, and most preferably by at least 75%, 80%, 90%, 95% or more. The double stranded RNA or antisense RNA is at least 10, 20, or 30 nucleotides in length. Other preferred lengths include 40, 60, 85, 120, or more consecutive nucleotides that are complementary to a hnRNP A1, hnRNP A1^{B}, hnRNP A2, or hnRNP A2/B1 mRNA or DNA, or splice variants or isoforms thereof, and may be as long as a full-length hnRNP A1, hnRNP A1^{B}, hnRNP A2, or hnRNP A2/B1 gene, mRNA, or DNA, or splice variants or isoforms thereof. The double stranded nucleic acid may contain a modified backbone, for example, phosphorothioate, phosphorodithioate, or other modified backbones known in the art, or may contain non-natural internucleoside linkages. In one preferred embodiment, short 19, 20, 21, 22, 23, 24, or 25 nucleotide double stranded RNAs are used to down regulate the expression or biological activity of hnRNP A1, hnRNP A1^{B}, hnRNP A2, or hnRNP A2/B1 expression and that may be used, for example, as therapeutics to treat a variety of neoplasias. Such RNAs are effective at down-regulating gene expression in mammalian tissue culture cell lines (Elbashir et al., Nature 411: 494-498, 2001, hereby incorporated by reference). The further therapeutic effectiveness of this approach in mammals was demonstrated in vivo by McCaffrey et al. (Nature 418: 38-39. 2002).

By “small interfering RNA” or “siRNA” is meant an isolated RNA molecule, preferably greater than 10 nucleotides in length, more preferably greater than 15 nucleotides in length, and most preferably 18, 19, 20, 21, 22, 23, 24, 25, 26, 27, 28, 29, 30, 35, 45, 50 or more nucleotides in length that is used to identify a target gene or mRNA to be degraded. A range of 19-25 nucleotides is the most preferred size for siRNAs. siRNAs can also include short hairpin RNAs (shRNA) in which both strands of an siRNA duplex are included within a single RNA molecule. Double-stranded siRNAs generally consist of a sense and anti-sense strand. Single-stranded siRNAs generally consist of only the anti-sense strand that is complementary to the target gene. siRNA includes any form of RNA, preferably dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the 21 to 23 nucleotide RNA or internally (at one or more nucleotides of the RNA). In a preferred embodiment, the RNA molecule contains a 3′hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incoporation) can be found in the published U.S. application publication number 20040019001 (see Summary of the Invention section). Collectively, all such altered RNAs are referred to as modified siRNAs. In particular embodiments, siRNAs can be synthesized or generated by processing longer double-stranded RNAs, for example, in the presence of the enzyme dicer under conditions in which the dsRNA is processed to RNA molecules of about 18 to about 25 nucleotides.

siRNAs of the present invention need only be sufficiently similar to natural RNA such that it has the ability to mediate RNAi. As used herein “mediate RNAi” refers to the ability to distinguish or identify which RNAs are to be degraded. Preferably, RNAi is capable of decreasing the expression of hnRNP A1, hnRNP A1^{B}, hnRNP A2, or hnRNP A2/B1, or splice variants or isoforms thereof, in a cell by at least 10%, 20%, 30%, or 40%, more preferably by at least 50%, 60%, or 70%, and most preferably by at least 75%, 80%, 90%, 95% or more. In one preferred embodiment, short 21, 22, 23, 24, or 25 nucleotide double stranded RNAs are used to down regulate hnRNP A1, hnRNP A1^{B}, hnRNP A2, or hnRNP A2/B1 expression (Elbashir et al., Nature 411: 494-498, 2001).

By “shRNA” is meant an RNA comprising a duplex region complementary to an mRNA. For example, a short hairpin RNA (shRNA) may comprise a duplex region containing nucleotides, where the duplex is between 19 and 29 bases in length, and the strands are separated by a single-stranded 3, 4, 5, 6, 7, 8, 9, or 10 base linker region. Optimally, the linker region is 6 bases in length.

By “subject” is meant a mammal, including, but not limited to, a human or non-human mammal, such as a bovine, equine, canine, ovine, or feline.

By “substantially identical” is meant a polypeptide or nucleic acid exhibiting at least 75%, but preferably 85%, more preferably 90%, most preferably 95%, or even 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, by “substantially identical” is also meant “substantially complementary.” For nucleic acids, the length of comparison sequences will generally be at least 10, 20, 30, 40, 50, 60, 70, 80, 90, 100, 120, 150, 200, or more nucleotides.

Sequence identity is typically measured using publicly available computer programs. Computer program methods to determine identity between two sequences include, but are not limited to, the GCG program package (Devereux et al., Nucleic Acids Research 12: 387, 1984), BLASTP, BLASTN, and FASTA (Altschul et al., J. Mol. Biol. 215: 403 (1990). The well-known Smith Waterman algorithm may also be used to determine identity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). These software programs match similar sequences by assigning degrees of homology to various substitutions, deletions, and other modifications. Conservative substitutions for amino acid comparisons typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “telomerase” is meant the enzyme responsible for the addition of TTAGGG repeats to the ends of telomeres.

By “telomere” is meant the end section of a eukaryotic chromosome, composed of several hundred terminal repeats of the sequence TTAGGG.

By a “therapeutic amount” is meant an amount of a compound, alone or in combination with known therapeutics that is sufficient to inhibit neoplasia growth, progression, or metastasis in vivo. The effective amount of an active compound(s) used to practice the present invention for therapeutic treatment of neoplasms (i.e., neoplasia) varies depending upon the manner of administration, the age, body weight, and general health of the subject. Ultimately, the attending physician or veterinarian will decide the appropriate amount and dosage regimen. An effective amount of an hnRNP A1, hnRNP A1^{B}, hnRNP A2, or hnRNP A2/B1 therapeutic for the treatment of neoplasia is as little as 0.005, 0.01, 0.02, 0.025, 0.05, 0.075, 0.1, 0.133 mg per dose, or as much as 0.15, 0.399, 0.5, 0.57, 0.6, 0.7, 0.8, 1.0, 1.25, 1.5, 2.0 or 2.5 mg per dose. The dose may be administered once a day, once every two, three, four, seven, fourteen, or twenty-one days. The amount administered to treat neoplasia is based on the activity of the therapeutic compound. It is an amount that is sufficient to effectively reduce cell proliferation, tumor size, neoplasia progression, or metastasis. It will be appreciated that there will be many ways known in the art to determine the therapeutic amount for a given application. For example, the pharmacological methods for dosage determination may be used in the therapeutic context.

Other features and advantages of the invention will be apparent from the following description of the preferred embodiments thereof, and from the claims.

### BRIEF DESCRIPTION OF DRAWINGS

FIG. 1 shows a Western blot of hnRNP A1 and hnRNP A2 expression in siRNA transfected HeLaS3 cells. Cells from the cervical carcinoma HeLaS3 cell line were seeded in 6-well plates (65,000 cells/well) and were transfected at 24 and 48 hours. Control samples were treated with oligofectamine in the absence of siRNA. Cells were collected 96 hours after the first transfection. Ponceau S-staining of the nitrocellulose membrane was used to confirm that equal amounts of protein were loaded in each lane. The hnRNP A1, hnRNP A2 proteins, and their respective spliced isoforms A1^{B} and B1 were quantitated with the polyclonal anti-A1/A2 antibody (see Patry et al., Cancer Res., 63: 7679-7688, 2003). A1#1-A1#7: sense and antisense siRNAs targeting the human hnRNP A1 mRNA; A2#1-A2#5: sense and antisense siRNAs targeting the human hnRNP A2 mRNA; A1#1M: control siRNA containing a mismatched version of A1#1, control: lipofectamine without siRNA. (Note: these abbreviations have this meaning throughout the figures).

FIG. 2 is a histogram showing cell growth of siRNA transfected HeLaS3 cells. The siRNA targeted either hnRNP A1 (A1#1-A1#7, A1 mismatched control A1#M) or hnRNP A2 (A2#1-A2#5). 96 hours post-transfection, adherent cells were photographed and both adherent and floating cells were harvested and counted. Cell viability was evaluated by trypan blue dye exclusion. The hatched area indicates that cells show the characteristic morphology associated with apoptosis.

FIG. 3 shows micrographs of siRNA-transfected HeLaS3 cells under phase contrast microscopy (200× magnification). Control: lipofectamine; siA1: siRNA targeting hnRNP A1; siA1M: mismatch hnRNP A1 control; siA2: siRNA targeting hnRNP A2. (Note: these abbreviations have this meaning throughout the figures).

FIG. 4A upper panel shows a Western blot analysis with a monoclonal antibody that recognizes both the 33 kDa inactive pro-caspase-3 as well as the activated 20 kDa form found in apoptotic cells. HeLaS3 cells were transfected as described above and cells were harvested 96 hours after the first transfection. FIG. 4A lower panel shows a western analysis performed on the same protein samples with an antibody that recognizes the PARP enzyme, which is a substrate for the activated caspase-3.

FIG. 4B shows a TUNEL assay on HeLaS3 cells treated with lipofectamine (control) a combination of siRNAs targeting hnRNP A1 and A2 (siA1+siA2), a mismatch control combination (siA1M+siA2) or staurosporin.

FIG. 4C shows DNA content in siRNA-treated cells that were fixed and stained with propidium iodide prior to DNA content analysis by cytometry. “n” refers to the haploid DNA content. Note that the appearance of subG₁ DNA associated with apoptosis is seen in HeLa S3 cells.

FIG. 5A shows the results of an oligonucleotide ligation assay to measure the length of telomeric single-stranded extensions. Seventy-two hours after the first transfection, HeLaS3 cells were harvested and cellular DNA was extracted. The oligonucleotide ligation assay was performed using 5 μg of cellular DNA and the ligation products were resolved on a sequencing gel, detected by autoradiography. The gel was scanned, and the histogram beside the gel shows the band intensity of the scanned image. Lane 1:combination of A1#1 and A2#1; lane 2: A1#1M and A2.

FIG. 5B is a graph showing the quantitation of the oligonucleotide ligation assay of FIG. 5A. Similar results were seen 48 hours after the first transfection. The image was analyzed using Quantity One quantification software™ (Bio-Rad). The value of the intensity of each band of the ligation products ladder was normalized by dividing for the number of concatenated oligonucleotide probes in the band. This value was then normalized to the total intensity and plotted as relative frequency of the 3′-overhang length.

FIG. 5C provides a measurement of the telomeric single-stranded 3′-overhang in HeLaS3 cells treated with staurosporine (lane 2) for 24 hours or with DMSO as control (lane 1). The gel was scanned, and the histogram beside the gel shows the band intensity of the scanned image.

FIG. 5D provides a quantitation of the telomeric probe ligation products of the assay ligation assay shown in FIG. 5C. The image was analyzed using Quantity One quantification software™ (Bio-Rad). The value of the intensity of each band of the ligation products ladder was normalized by dividing for the number of concatenated oligonucleotide probes in the band. This value was then normalized to the total intensity and plotted as relative frequency of the 3′-overhang length.

FIG. 6 is a histogram showing the effect of varying siRNA concentrations targeting hnRNP A1 and hnRNP A2 on HeLaS3 cell viability. HeLaS3 cells were seeded in 6-well plates (65,000 cells/well) 24 hours before transfection. Cells were transfected twice with the indicated concentrations of siRNA and cell viability analysis was performed using Trypan Blue dye exclusion assays 96 hours after transfection. NT: Non-transfected; control: lipofectamine without siRNA; lamin A/C.

FIG. 7A is a graph showing cell viability measurements of HeLaS3 cells at various time points after siRNA transfection. HeLaS3 cells were seeded in 6-well plates (65,000 cells/well) 24 hours before transfection. Cells were transfected with siRNA at 24 hours and 48 hours and cell viability analysis was performed using Trypan Blue exclusion assays at 72 hours, 96 hours, 120 hours, and 144 hours after the first transfection.

FIG. 7B shows hnRNPA1 and hnRNP A2 protein expression in HeLaS3 cell extracts assayed after siRNA transfection. Extracts from 40,000 cells transfected as above were harvested at 72 hours, 96 hours, 120 hours, and 144 hours after transfection, separated by SDS-PAGE, transferred to a membrane, and immunoblotted using a polyclonal antibody against A1/A2/A1^{B}/B1. Oligofect: control transfection without siRNA.

FIG. 8 is a Western blot showing the impact of treatment with siRNAs on hnRNP A1 and hnRNP A2 expression in a HCT116 neoplastic cell line.

FIG. 9A is a histogram showing the effect of siRNA targeting hnRNP A1 and A2 on HCT116 colorectal carcinoma cell line on cell growth. The bottom portion shows a western analysis of the A1/A2 expression.

FIG. 9B shows photomicrographs of siRNA treated HCT116 cells. Seventy-two hours post transfection, cells were harvested and processed to determine the impact on hnRNP A1 hnRNP A2 expression on the phenotype.

FIG. 10 is a Western blot showing the impact of treatment with siRNAs on hnRNP A1 and hnRNP A2 expression in the HT1080 fibrosarcoma neoplastic cell line.

FIG. 11A is a histogram and Western blot showing the effect of siRNA transfection on cell growth and hnRNP A1 and hnRNP A2 expression in HT1080 cells. siA1M+siA2: mismatch control combination; control: lipofectamine treatment without siRNA present; siA1+siA2: siRNA combination targeting hnRNP A1 and hnRNP A2.

FIG. 11B shows photomicrographs of HT1080 cells transfected with the indicated siRNAs. siA1M+siA2: mismatch control combination; control is lipofectamine treatment without siRNA present; siA1+siA2: siRNA combination targeting hnRNP A1 and hnRNP A2.

FIG. 12A is a histogram showing the effect of siRNA targeting hnRNP A1 and A2 on the growth of the MCF-7 breast neoplastic cell line. The bottom portion shows a western analysis of the hnRNP A1 and A2 expression.

FIG. 12B shows photomicrographs of siRNA treated MCF-7 cells. Seventy-two hours post transfection, cells were harvested and processed to determine the impact on hnRNP A1 hnRNP A2 expression on the phenotype.

FIG. 13A is a histogram and Western blot showing the effect of siRNA transfection on cell growth and hnRNP A1 and hnRNP A2 expression in CCD-18Co cells.

FIG. 13B shows photomicrographs of CCD-18Co cells transfected with the indicated siRNAs. siA1M+siA2: mismatch control combination; Control is lipofectamine treatment without siRNA present; siA1+siA2: siRNA combination targeting hnRNP A1 and hnRNP A2.

FIG. 14A is a histogram and Western blot showing the effect of siRNA transfection on cell growth and hnRNP A1 and hnRNP A2 expression in mortal BJ cells.

FIG. 14B shows photomicrographs of mortal BJ cells transfected with the indicated siRNAs.

FIG. 15A is a graph and Western blot showing the effect of siRNA transfection on cell growth and hnRNP A1 and hnRNP A2 expression in HIEC cells.

FIG. 15B shows photomicrographs of immortalized HIEC cells transfected with the indicated siRNAs.

FIG. 16A is a graph and western blot showing the effect of siRNA transfection on cell growth and hnRNP A1 and hnRNP A2 expression in immortalized BJ-TIELF cells.

FIG. 16B shows photomicrographs of immortalized BJ-TIELF cells transfected with the indicated siRNAs.

FIG. 17 shows DNA content analysis after RNAi on BJ-TIELF cells.

FIG. 18 is a table showing the effects of RNAi on hnRNP A1 and hnRNP A2 expression in various cell lines.

FIG. 19A shows a micrograph of hnRNP A1 and hnRNP A2 expression in lung tissue from a normal patient.

FIG. 19B shows a micrograph of hnRNPA1 and hnRNPA2 expression from a patient with lung adenocarcinoma.

FIG. 19C shows immunohistochemistry analysis of hnRNP A1 and hnRNP A2 expression in a pancreatic tissue from a normal patient.

FIG. 19D shows immunohistochemistry analysis of hnRNP A1 and hnRNP A2 expression in a pancreatic tissue from a patient with pancreatic adenocarcinoma. Magnification, 40×.

FIG. 20A is a western blot showing the effect of siRNA treatment on A1/A2 expression (using a rabbit polyclonal anti-A1/A2 antibody) in mouse testicular embryonic carcinoma F9 cells 72 hours after the first transfection.

FIG. 20B is a graph showing the effect of siRNA treatment on cell growth in mouse F9 cells. Growth is compared to untreated cells (control) which are given a value of 1 (RU: Relative Unit). The white histogram indicates that cells displayed an altered morphology.

FIG. 20C is a graph showing the effect of siRNA treatment on mouse F9 cells after transfection with a larger collection of siRNAs 72 hours after transfection. Data is presented as in FIG. 20B.

FIG. 20D is a series of photomicrographs showing phase contrast microscopy (×200 magnification) of F9 cells treated with siRNAs.

FIG. 21A is a western blot showing the effect of siRNA treatment on A1/A2 expression (using a rabbit polyclonal anti-A1/A2 antibody) in 4T1, J774A.1 and P19 mouse neoplastic cell lines 72 hours after the first transfection.

FIG. 21B is a graph showing the effect of siRNA treatment on cell growth in 4T1, J774A.1 and P19 mouse neoplastic cell lines (relative to control). The white area indicates that cells show an altered morphology.

FIG. 21C is a series of photomicrographs showing phase contrast microscopy (×200 magnification) of 4T 1, P19 and J774A.1 cells treated with siRNAs.

FIG. 22A is a western blot showing the effect of siRNA treatment on activated caspase-3expression (using an anti-activated caspases-3 antibody) in mouse F9 cells 72 hours after the beginning of each treatment.

FIG. 22B shows DNA content analysis of the siRNA treated cells. siRNA-treated cells were fixed and stained with propidium iodide before DNA content analysis by cytometry. n refers to the haploid DNA content.

FIG. 23A is a western blot showing the effect of siRNA treatment on A1/A2 expression in NIH/3T3 cells and mouse embryonic fibroblasts (MEF) 72 hours after the first transfection.

FIG. 23B is a graph showing the effect of various siRNA treatments on cell growth (relative to control).

FIG. 23C shows photomicrographs of cells treated with siRNAs using phase contrast microscopy (×200 magnification).

FIG. 23D shows DNA content analysis of the siRNA treated cells. n refers to the haploid DNA content.

FIG. 24A is a graph showing the effect of siRNA treatment and mouse hnRNP A1 expression on cell growth in HeLA S3 cells 96 hours after the first transfection.

FIG. 24B is a western blot showing the effect of siRNA treatment on A1 and A2 expression 96 hours after the first transfection. The expression of transfected mouse myc-hnRNPA1 using an anti-myc antibody is shown in the bottom panel. Further analyses indicated that myc-A1 co-migrates with the low abundance human B1 protein. The asterisk (*) indicates the position of the myc-A1 and B1 proteins. Based on the intensity of this band when all human A and B proteins are targeted (hA1-1+hmA2-1), we estimate that myc-A1 may represent as much as 50% of the total amount of A1 proteins in the untreated HeLa S3 cell clone.

FIG. 24C is a series of photomicrographs showing phase contrast microscopy (×200 magnification) of cells treated with siRNAs.

FIG. 24D is a western blot showing the effect of siRNA treatment on the expression of activated caspase-3.

### DETAILED DESCRIPTION OF THE INVENTION

The invention provides methods and compositions for treating and preventing neoplasia.

As reported in more detail below, we have discovered that inhibiting expression of hnRNP A1 and hnRNP A2, or alternatively spliced isoforms such as A1^{B} and B1, respectively, using RNA interference or antisense promotes rapid apoptotic cell death specifically in neoplastic cells.

We used RNA interference mediated by small interfering RNAs (siRNAs) to reduce levels of hnRNP A1 and hnRNP A2 proteins simultaneously, and the alternatively spliced isoforms A1^{B} and B1, respectively, in human and mouse neoplastic cell lines. This treatment promoted specific and rapid cell death by apoptosis in cell lines derived from cervical, colon, breast, ovarian and brain cancer. Neoplastic cell lines that lack p53 or that express a defective p53 protein were also sensitive to an siRNA-mediated decrease in hnRNP A1 and hnRNP A2 expression. Remarkably, comparable decreases in the expression of hnRNP A1 and hnRNP A2 in several mortal human and mouse fibroblastic and epithelial cell lines did not elicit cell death demonstrating tumor specificity. We have also demonstrated that the expression of mouse A1 cDNA protects human HeLa cells from apoptosis when human A1 and A2 proteins are targeted by RNA interference. These results establish A1 and A2 as specific proteins required for the viability of transformed murine and human cells and as such, they can be selectively targeted by the novel cancer therapeutics described herein.

Since A1B is an alternatively spliced isoform of A1 and has several identical exons, a preferred antisense nucleobase oligomers or siRNA molecule targets the common regions, thereby downregulating the expression of both the A1 and A1B genes. Similarly, since B1 is an alternatively spliced isoform of A2 and has several identical exons, a preferred antisense nucleic acid molecule or siRNA molecule targets the common regions thereby downregulating the expression of both the A2 and B1 genes.

### EXAMPLES

We examined the relationship between hnRNP A1 and hnRNP A2 expression and different types of human cancers. In addition, we determined the effect of alterations in hnRNP A1 and/or A2 protein levels on the growth of neoplastic and normal mortal cell lines using RNA interference (RNAi) to reduce the expression of hnRNP A1 and hnRNP A2 proteins in both human and mouse cell lines.

Our results on the expression profile of A1 and A2 identified these proteins as potential markers for many types of tumors. Most importantly, we showed that a combined reduction in hnRNP A1 and A2 expression promoted apoptosis in all neoplastic cell lines tested. A similar decrease in hnRNP A1 and hnRNP A2 protein levels in normal mortal cell lines had no significant effect on cell growth. The specificity of cell death mediated by siRNA targeting hnRNPA1 and hnRNP A2 was demonstrated by showing that the mouse hnRNPA1 protein protects human neoplastic cells from apoptosis when these cells sustain a reduction in human hnRNPA1 and hnRNP A2 proteins. Without being tied to a particular model, our results demonstrate that inhibiting hnRNP A1 and hnRNP A2 expression is a powerful and specific approach to prevent or inhibit the growth of neoplastic cells.

### Example 1

### Effects of RNAi on HeLaS3 Cell Growth and Protein hnRNP A1 and hnRNP A2 RNAi in HeLaS3 Cells

If hnRNP A1 and hnRNP A2 proteins are involved in the formation of a telomeric cap, inhibiting their expression should result in uncapping, cell growth arrest, and rapid cell death. To test this hypothesis, we needed to promote a specific reduction in the level of A1 and/or A2 proteins in human neoplastic cells. We accomplished this using siRNAs to carry out RNA interference assays.

Optimal conditions for siRNA transfection were identified using a fluorescent oligonucleotide and siRNA complementary to lamin A/C in HeLaS3 cells. We designed a variety of 19 base pair double-stranded RNAs containing a 2-nucleotide extension at the 3′ end and corresponding to portions of the A1 and A2 mRNAs. The sequences of specific siRNAs are provided below.

| | |
|---|---|
| A1#1: | |
| 5′-UGGGGAACGCUCACGGACUdTdT-3′ | (SEQ ID NO: 1) |
| 3′-dTdTACCCCUUGCGAGUGCCUGA-5′ | (SEQ ID NO: 2) |
| A1#1M: | |
| 5′-UGGGGAACCGUCACGGACUdTdT-3′ | (SEQ ID NO: 3) |
| 3′-dTdTACCCCUUGGGAGUGCCUGA-5′ | (SEQ ID NO: 4) |
| A1#2: | |
| 5′-UGAGAGAUCCAAACACCAAdTdT-3′ | (SEQ ID NO: 5) |
| 3′-dTdTACUCUCUAGGUUUGUGGUU-5′ | (SEQ ID NO: 6) |
| A1#3: | |
| 5′-GCGCUCCAGGGGCUUUGGGdTdT-3′ | (SEQ ID NO: 7) |
| 3′-dTdTCGCGAGGUCCCCGAAACCC-5′ | (SEQ ID NO: 8) |
| A1#4: | |
| 5′-UCGAAGGCCACACAAGGUGdTdT-3′ | (SEQ ID NO: 9) |
| 3′-dTdTAGCUUCCGGUGUGUUCCAC-5′ | (SEQ ID NO: 10) |
| A1#5: | |
| 5′-AUCAUGACUGACCGAGGCAdTdT-3′ | (SEQ ID NO: 11) |
| 3′-dTdTUAGUACUGACUGGCUCCGU-5′ | (SEQ ID NO: 12) |
| A1#6: | |
| 5′-CUUUGGUGGUGGUCGUGGAdTdT-3′ | (SEQ ID NO: 13) |
| 3′-dTdTGAAACCACCACCAGCACCU-5′ | (SEQ ID NO: 14) |
| A1#6M: | |
| 5′-CUUUGGUGUGGGUCGUGGAdTdT | (SEQ ID NO: 29) |
| 3′ dTdTGAAACCACACCCAGCACCU | (SEQ ID NO: 30) |
| A1#7: | |
| 5′-UUUUGGAGGUGGUGGAAGCdTdT-3′ | (SEQ ID NO: 15) |
| 3′-dTdTAAAACCUCCACCACCUUCG | (SEQ ID NO: 16) |
| A2#1: | |
| 5′-GCUUUGAAACCACAGAAGAdTdT-3′ | (SEQ ID NO: 17) |
| 3′-dTdTCGAAACUUUGGUGUCUUCU-5′ | (SEQ ID NO: 18) |
| A2#2: | |
| 5′-CCACAGAAGAAAGUUUGAGdTdT-3′ | (SEQ ID NO: 19) |
| 3′-dTdTGGUGUCUUCUUUCAAACUC-5′ | (SEQ ID NO: 20) |
| A2#3: | |
| 5′-GAAGCUGUUUGUUGGCGGAdTdT-3′ | (SEQ ID NO: 21) |
| 3′-dTdTCUUCGACAAACAACCGCCU-5′ | (SEQ ID NO: 22) |
| A2#4: | |
| 5′-AUUUCGGACCAGGACCAGGdTdT-3′ | (SEQ ID NO: 23) |
| 3′-dTdTUAAAGCCUGGUCCUGGUCC-5′ | (SEQ ID NO: 24) |
| A2#5: | |
| 5′-CUUUGGUGGUAGCAGGAACdTdT-3′ | (SEQ ID NO: 25) |
| 3′-dTdTGAAACCACCAUCGUCCUUG-5′ | (SEQ ID NO: 26) |

Each of these RNAs was tested as follows.

Double-stranded siRNAs complementary to a portion of A1 or A2 were individually introduced into HeLaS3 cells by performing two successive transfections with an A1 and an A2 siRNA (20 nM). The second transfection was performed 24-hours after the first. Seven different siRNAs complementary to a portion of A1 and five siRNAs complementrary to a portion of A2 were tested. Control samples were treated with oligofectamine in the absence of an siRNA. As an additional negative control, the siRNA A1-1M was used. This control contained a mismatched version of A1-1 having a mutation at two adjacent positions (GC to CG).

Cells were counted after Trypan blue staining and cell growth was evaluated by calculating the number of cell divisions (expressed as the number of population doublings) 96 hours after the first transfection.

### Example 2

### hnRNP A1 and A2 Protein Expression in siRNA Transfected Cells

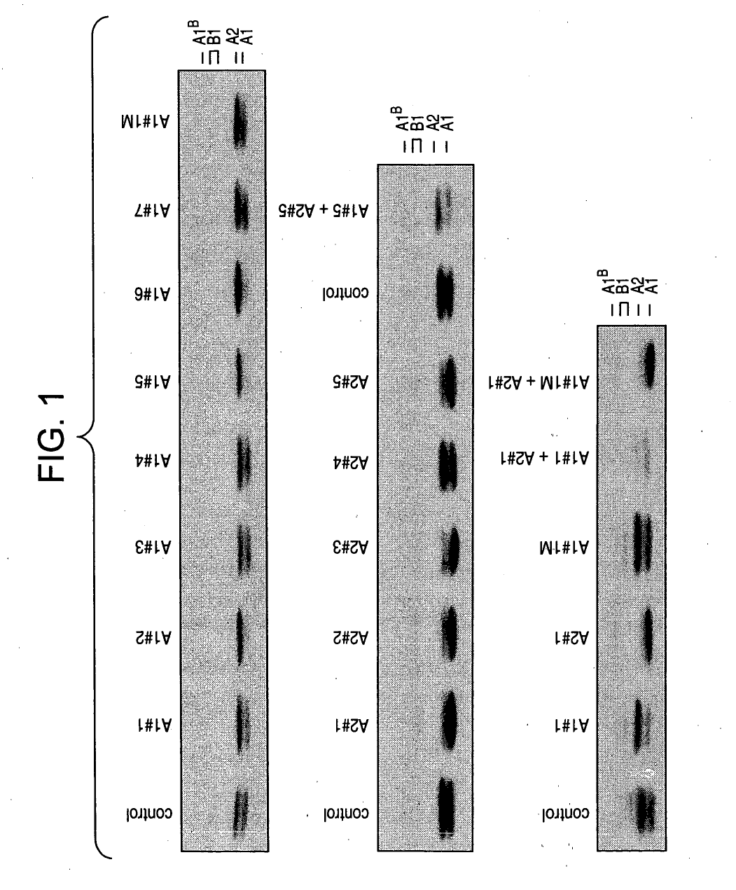

Ninety-six hours after the first transfection (as described in Example 1), total proteins were isolated and the abundance of A1 and A2 proteins was assessed by western analysis using a rabbit polyclonal antibody that binds A1, A2, and their lower abundance splice isoforms, A1^{B} and B1 (FIG. 1).

Protein extracts from cells transfected with siRNAs targeting either hnRNP A1 or hnRNP A2 (A1-1, A1-2, A1-5 and A1-6) showed a marked reduction in the protein expression level of A1. All siRNAs against A2, with the exception of A2-4, promoted a strong decrease in A2 protein level. siRNA A1-1M did not promote a reduction in hnRNP A1. Thus, we identified several siRNAs that reduced the expression of hnRNP A1 and A2.

### Example 3

### Cell Growth Assays in siRNA A1 and A2 Transfected Cells

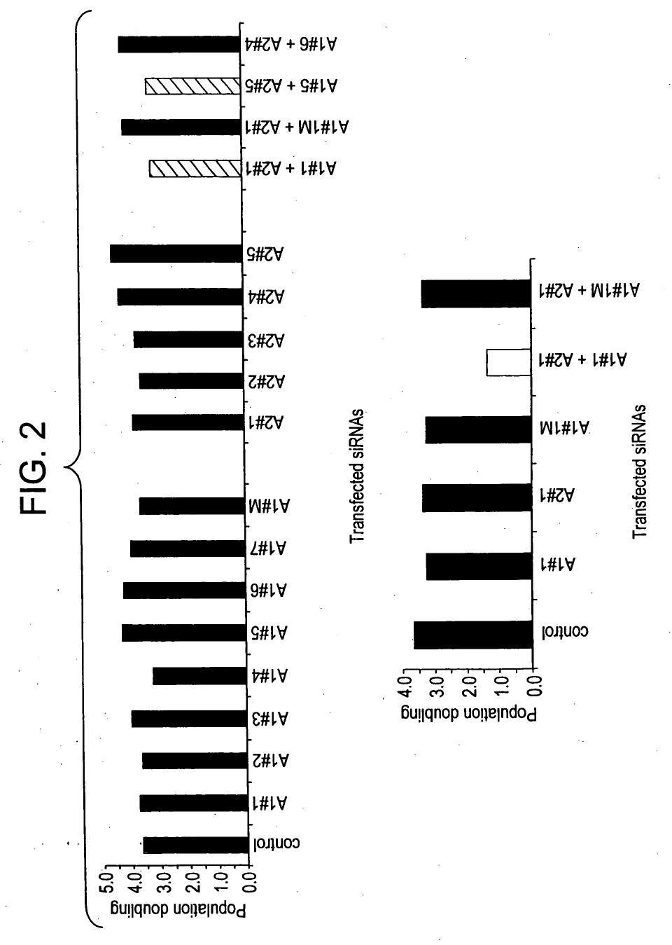

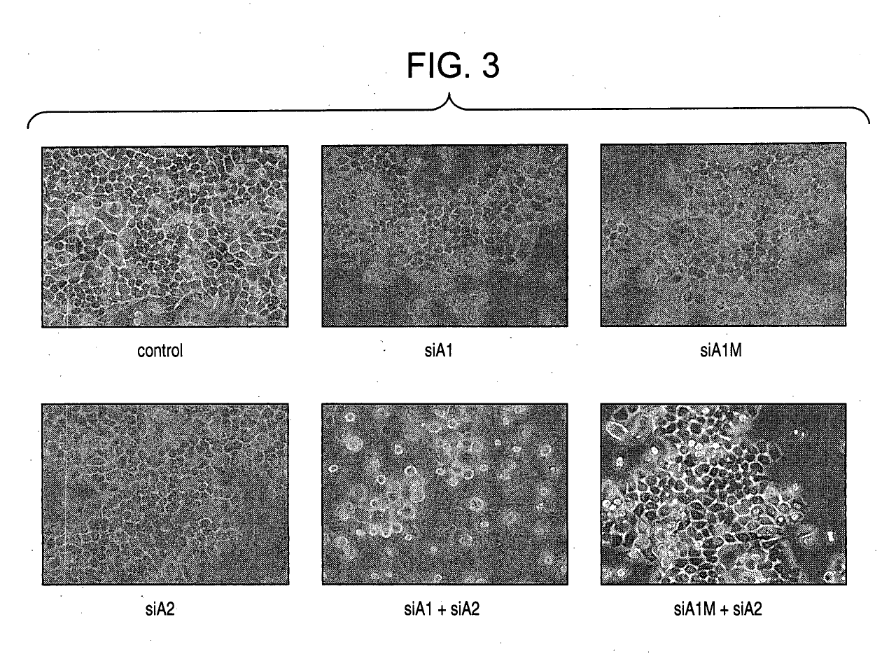

To determine the effect of siRNAs that target A1 and A2 affected cell growth in human cells (FIG. 2), we transfected HeLaS3 cells with individual siRNAs, combinations of siRNAs, and control mixtures. Adherent and non-adherent cells were collected and counted 96 hours after the first transfection. We also assessed gross cellular morphology by microscopic inspection (FIG. 3). Individual siRNAs that decreased either A1 or A2 expression levels did not affect cell growth nor did they change cell morphology.

Combinations of siRNAs that promoted a reduction in the abundance of both hnRNP A1 and A2 (siRNAs A1-1/A2-1 and A1-5/A2-5) affected cell growth and cell morphology. In fact, the morphology of cells treated with these combinations that targeted hnRNP A1 and hnRNP A2 resembled apoptotic cells. In some experiments, the reduction in cell growth was less apparent, but the majority of the cells examined were round and loosely adherent. We attribute the variations in cell growth between experiments to differences in the timing of cell death.

Trypan blue exclusion staining indicated that the majority of the cells treated with siRNA combinations targeting both hnRNP A1 and hnRNP A2 always produced increased numbers of dead cells relative to cells treated with individual siRNAs targeting hnRNP A1 or hnRNP A2. Pairs of siRNAs that affected only hnRNP A1 or hnRNP A2 did not elicit these effects (e.g. A1-6/A2-4). Likewise, the mismatch control siRNA (A1-1M/A2-1) pair, which promoted a decrease in hnRNP A2 protein levels, but did not produce a decrease in hnRNP A1 protein levels, did not affect cell growth and cell morphology. Thus, specific combinations of siRNAs that targeted both hnRNP A1 and hnRNP A2 (A1-1/A2-1 siRNA), were effective at reducing A1 and A2 protein expression and at promoting cell death. The experiment shown in FIG. 2 was conducted at a concentration of 80 nM for individual siRNA and a total concentration of 80 nM when pairs of siRNAs (40 nM of each) were used. This experiment was repeated many times (n>10) with identical results. Although mixtures of siRNAs at 20 nM were active, lower concentrations did not efficiently reduce cell viability. A fifty percent decrease in the level of hnRNP A1 and hnRNP A2 protein levels relative to untreated cells almost invariably promoted cell death. Treatment with individual siRNA targeting hnRNP A1 or hnRNP A2 had no effect on cell growth when tested at a concentration of 120 nM, 210 nM and 300 nM, 300 nM being the highest concentration tested.

We also tested HeLaS3 cells grown at low concentrations of serum. Under these conditions, the number of cell divisions for the control mixture remained low (less than 3 population doublings in 96 hours), but specific siRNA-induced cell death was as dramatic. The reduction in cell growth, the change in cell morphology and the results of differential staining for live cells using trypan blue all suggested that siRNAs combinations targeting both hnRNP A1 and hnRNP A2 promoted cell death.

### Example 4

### Apoptotic Assays in siRNA Transfected Cells

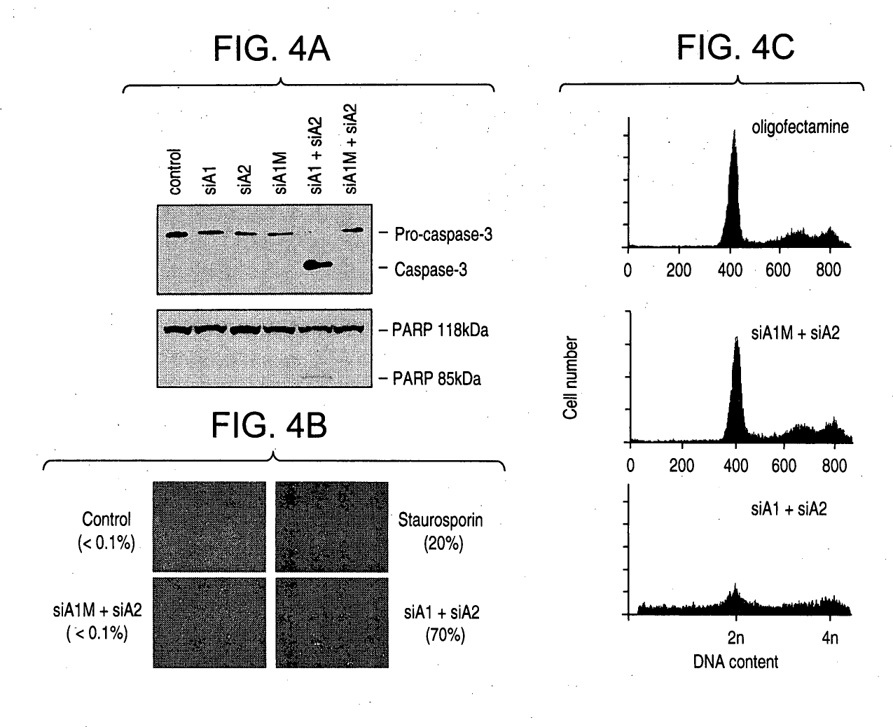

To confirm that this cell death was occurring by apoptosis, we carried out a variety of assays, including PARP, pro-caspase-3 cleavage, and DNA content analysis assays (FIGS. 4A-4C). The siRNA combination targeting both hnRNP A1 and hnRNP A2 (A1-1/A2-1) resulted in cell death by apoptosis as assayed by pro-caspase 3 protein cleavage.

DNA content analysis indicated that a characteristic subG1 increase due to DNA fractionation was observed with the siRNA combination that targeted hnRNP A1 and hnRNP A2 (A1-1/A2-1), but not with the siRNA mismatch combination control (A1-1M/A2-1) (FIG. 4C). We also carried out TUNEL assays (FIG. 4B) that specifically stain apoptotic cells. These assays indicated that more than 70% of the HeLa cells were TUNEL-positive when treated with the A1-1/A2-1 siRNAs. Less than 0.1% of cell treated with the control A1-1M/A2-1 siRNA combination (FIG. 4B) were TUNEL-positive. Thus, apoptotic analyses indicated that a reduction in hnRNP A1 and hnRNP A2 expression in HeLaS3 cells promoted apoptosis.

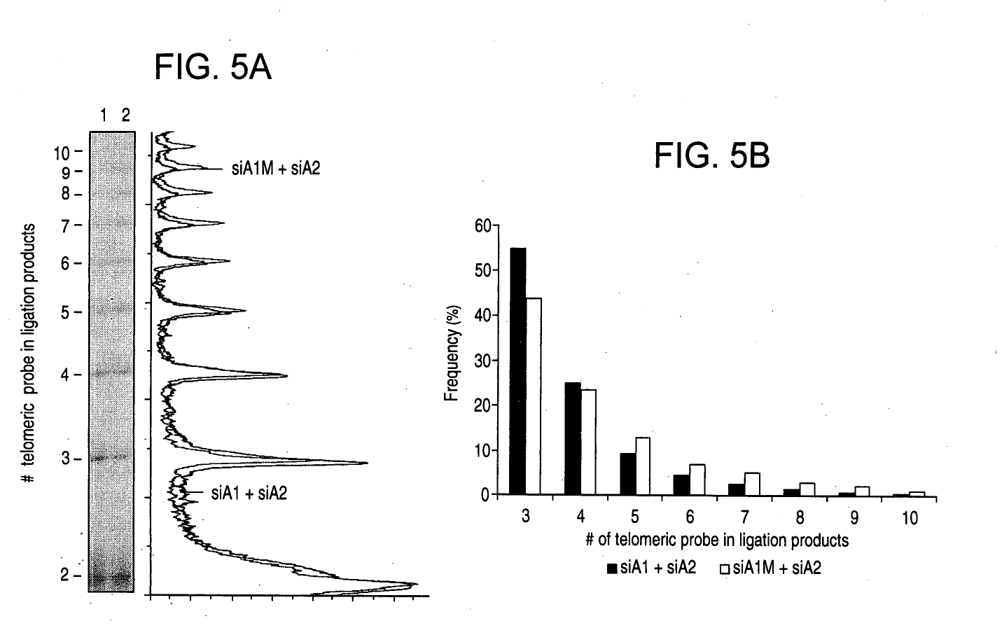

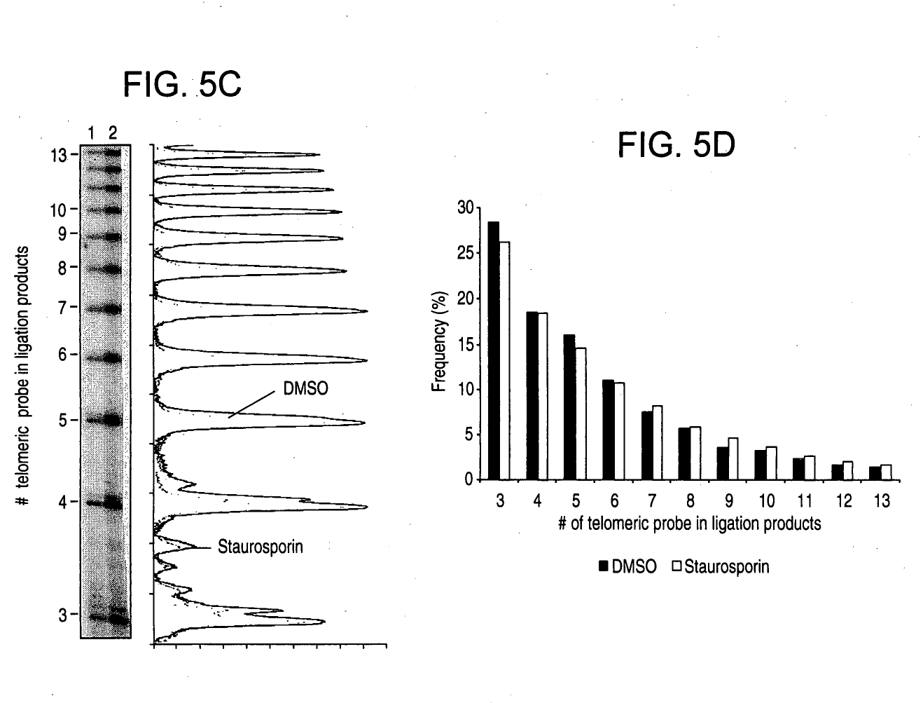

The rapid cell death elicited by siRNAs targeting hnRNP A1 and hnRNP A2 was consistent with these proteins functioning as telomeric capping proteins. If this is the case, one would predict that a reduction in hnRNP A1 and hnRNP A2 levels would result in a decrease in the length of single-stranded G-rich extension on telomeres. To determine whether the single-stranded extensions were shortened when hnRNP A1 and hnRNP A2 levels were reduced by siRNA treatment, we performed a telomere oligonucleotide ligation assay (T-OLA) (FIGS. 5A and 5B). This assay characterized the size distribution of G-rich extensions in HeLaS3 cells treated with siRNAs combinations targeting hnRNP A1 and A2 and control siRNAs. HeLaS3 cells treated for 72 hours with the siRNA combination targeting both hnRNP A1 and hnRNP A2 exhibited a difference in the size distribution of ligated telomeric oligonucleotides (FIG. 5A) relative to cells treated with the control siRNA mismatch control combination (A1-1M/A2-1). A decrease in hnRNP A1 and A2 expression was associated with shorter telomeric extensions (FIGS. 5C and 5D). The same result was observed at 48-hours post-transfection. Most importantly, we did not observe a similar change in the length of the G-rich extensions when HeLaS3 cells were treated for 48-hours with staurosporine, an inducer of apoptosis.

### Example 5

### Comparison of Varying Concentration of siRNA on RNAi Efficacy

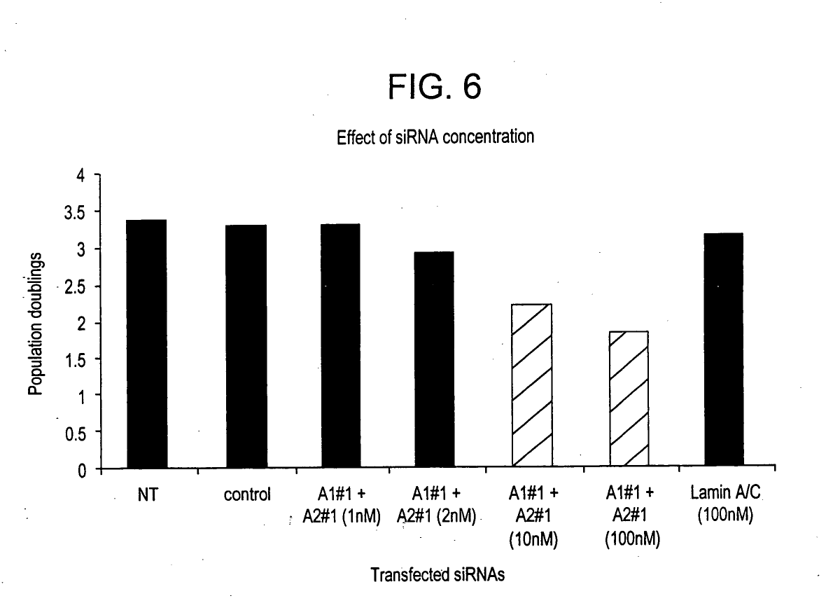

HeLa cells were seeded in 6-well plates (65,000 cells/well) and after 24-hours they were transfected with combinations of siRNA targeting both hnRNP A1 and hnRNP A2 (A1#1 and A2#1 or A1#2 and A2#1) using the methods described below. At 96-hours, Trypan blue dye exclusion assays for cell viability were performed. Final concentrations of siRNA of 1 nM and 2 nM were inefficient at reducing cell viability (FIG. 6). Final concentrations of siRNAs of 100 nM and 10 nM were approximately equivalent in their ability to reduce cell viability (FIG. 6; Note: the hatched area indicates that the cells presented an altered morphology characteristic of apoptotic cells). The 10 nM siRNA combination concentration was slightly less effective.

### Example 6

### Time Course of siRNA Treatment on HeLaS3 Cells

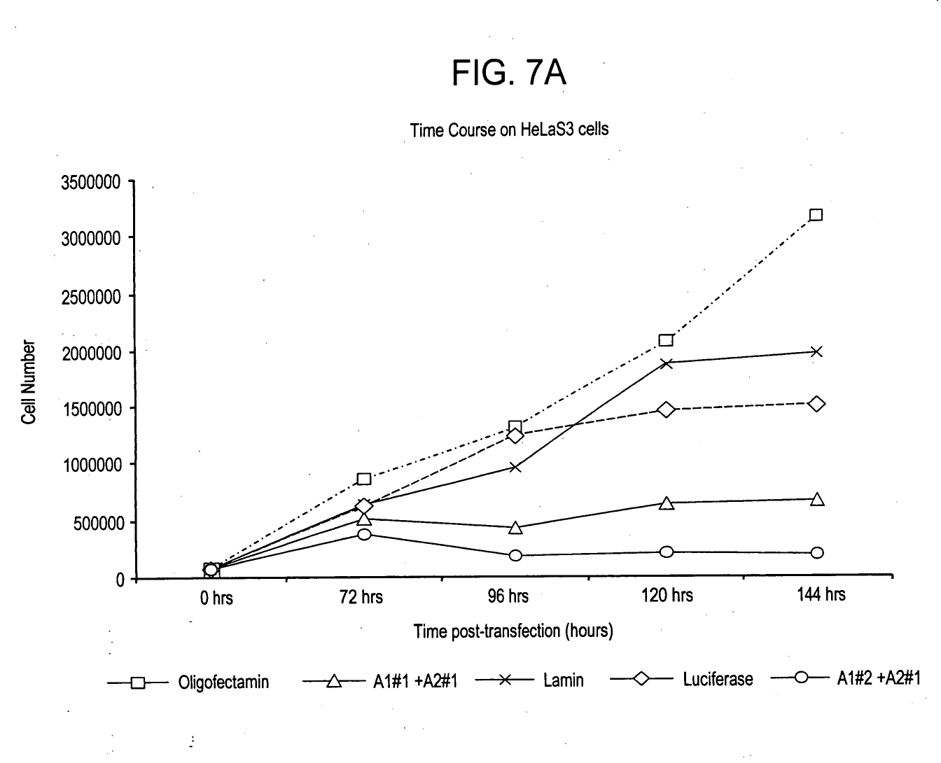

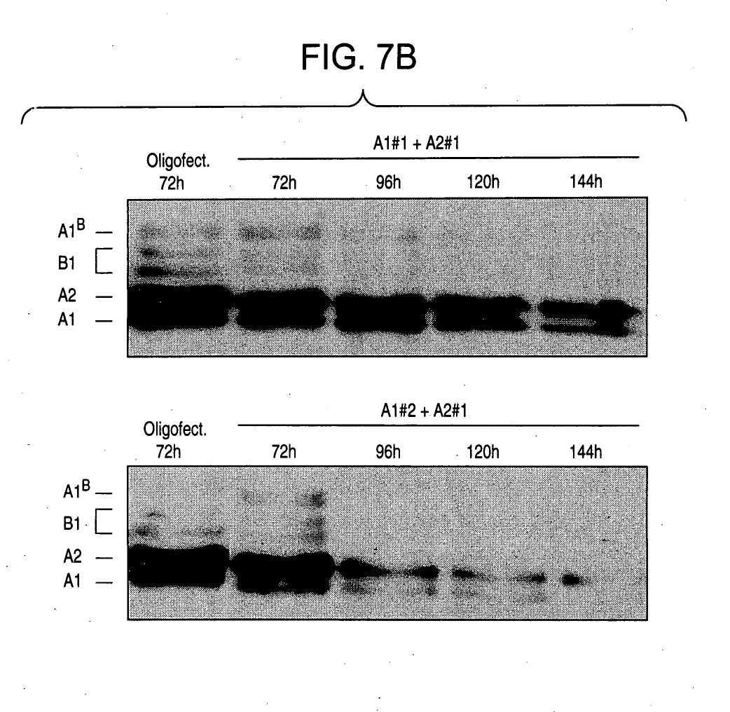

HeLaS3 cells were seeded in 6-well plates (65,000 cells/well) and were transfected at 24 and 48 hours with the indicated combinations of siRNA (80 nM). At each time point indicated, Trypan blue dye exclusion assays for cell viability were performed. Maximal cell death was seen 96 hours after the first transfection (FIG. 7A).

Whole cell extracts from 40,000 cells taken at the indicated time points were analyzed by western blotting using a polyclonal antibody against A1/A2/A1^{B}/B1. The extracts from cells transfected with siRNA combinations that targeted both hnRNP A1 and A2 (A1#1 and A2#1) showed reduced protein expression beginning at 72 hours with a maximal reduction achieved by 144 hours after the first transfection (FIG. 7B, top panel). The extracts from cells transfected with siRNA A1#2 and A2#1 showed reduced protein expression beginning at 72 hours and almost no detectable protein expression at 144 hours (FIG. 7B, lower panel). Ponceau S-staining of the nitrocellulose membrane was used in both conditions to confirm equal protein loading.

### Example 7

### hnRNP A1 and A2-Targeted RNAi Promotes Apoptosis in a Diverse Array of Neoplastic Cell Lines

The effectiveness of RNAi in reducing levels of A1 and A2 in HeLaS3 cells and their effect on cell viability was assayed in cell lines derived from a variety of human cancers.

Colorectal carcinoma

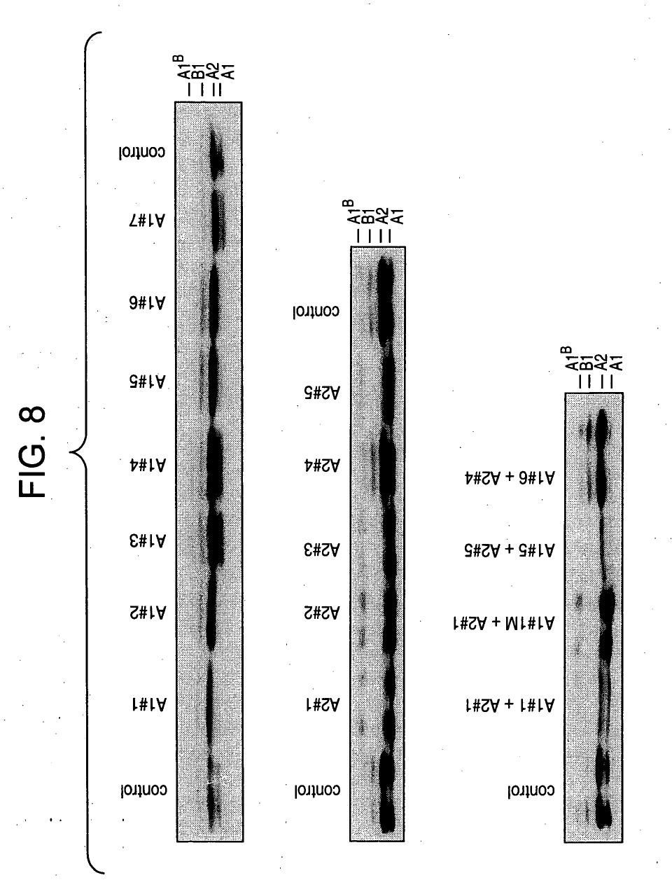

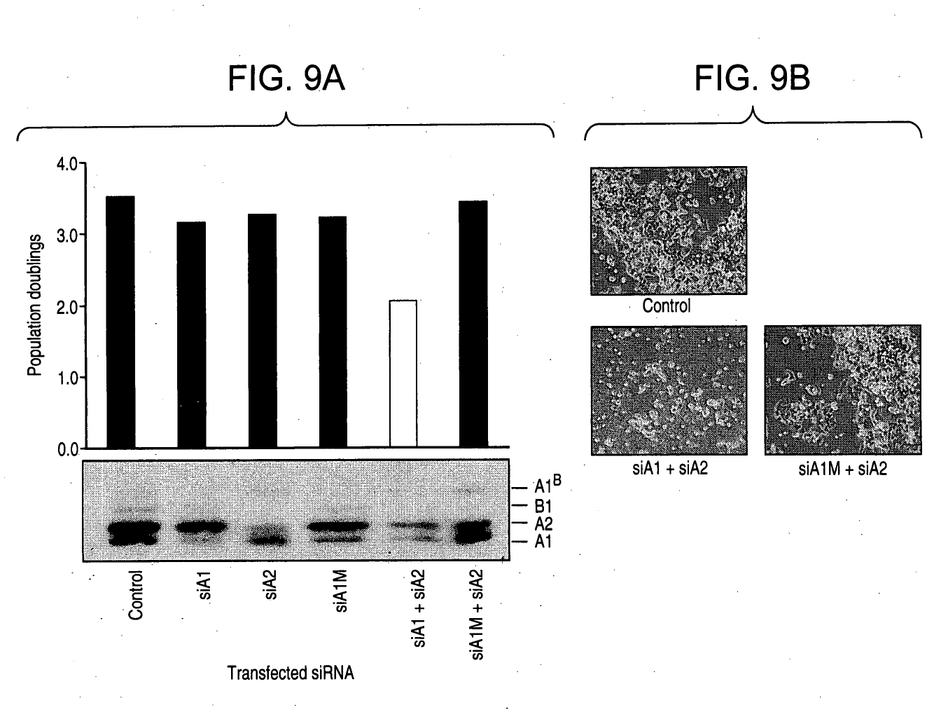

We first tested the effect of individual hnRNP A1 and A2 siRNAs, and combinations thereof, on the colorectal carcinoma cell line HCT116 (FIG. 8). Individual or combinations of siRNAs targeting hnRNP A1 and/or hnRNP A2 were applied twice to HCT116. Cell viability was measured at 72-hours post-transfection. Similar to what was observed for HeLaS3 cells, treatment with individual siRNAs promoted a reduction in the targeted protein (FIG. 8), but only the combinations of siRNAs targeting both hnRNP A1 and A2 reduced the growth and altered the morphology of HCT116 cells (FIGS. 9A and 9B). Cells transfected with the mismatched control combination A1-1M/A2-1, showed a reduction in A2, but they did not change morphology (FIG. 9B). The apoptotic phenotype was confirmed by testing for PARP and pro-caspase-3 cleavage. Thus, specific combinations of siRNAs targeting both hnRNP A1 and hnRNP A2 effectively inhibited A1 and A2 protein expression and promoted the death of HCT116 cells. Similar results were obtained with the colorectal carcinoma cell line HT29, which expresses a mutated p53. These results indicate that the siRNA hnRNP A1 and hnRNP A2-mediated apoptosis occurs independently of p53.

Fibrosarcoma

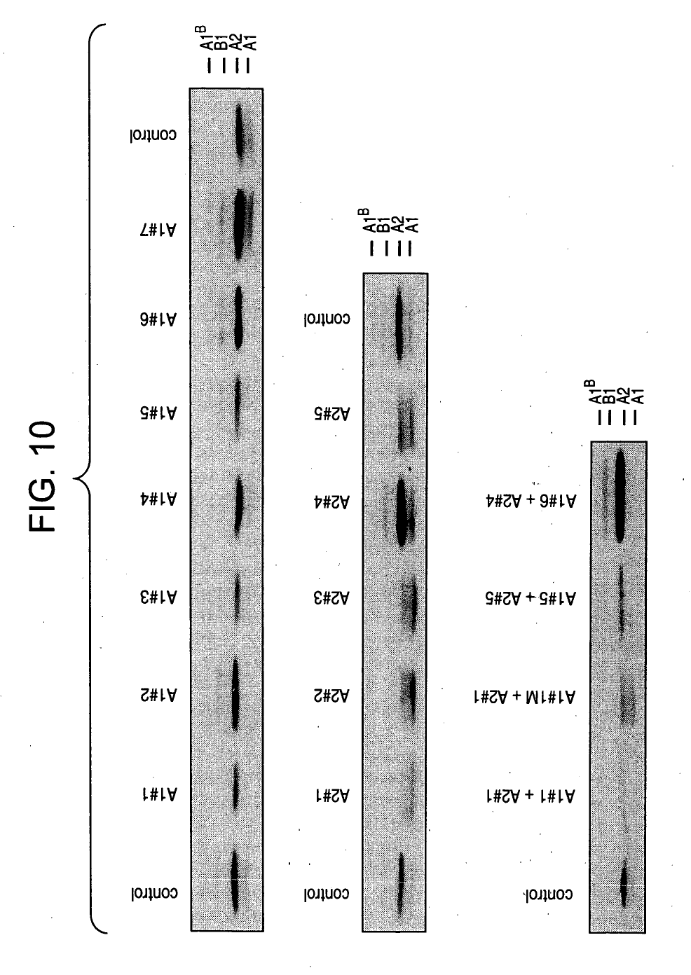

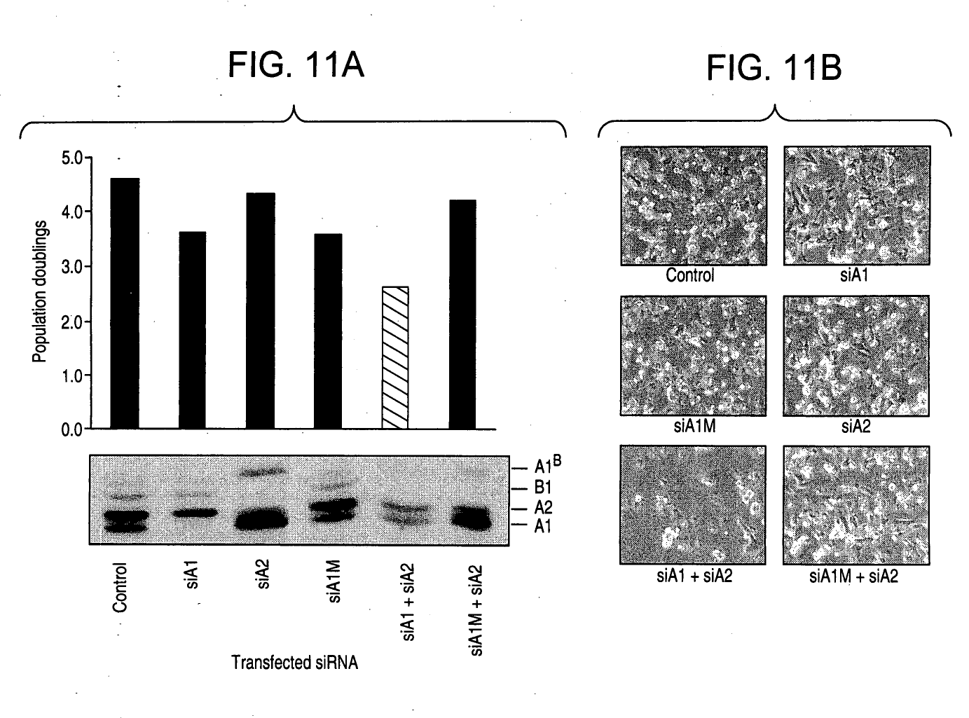

The effect of siRNA-mediated reduction in hnRNP A1 and A2 expression was also tested in the fibrosarcoma cell line HT1080 (FIG. 10). These assays were carried out as described for HCT116. The siRNA-mediated reduction in hnRNP A1 and A2 expression correlated with a reduction in protein expression (FIG. 10), cell growth (FIG. 11A) and a change in cell morphology that is characteristic of apoptosis (FIG. 11B). The DNA content analysis revealed an increase in cells in the subG1 category, indicative of apoptosis-mediated chromatin fractionation.

Breast Carcinoma, Ovarian Carcinoma, and Glioblastoma

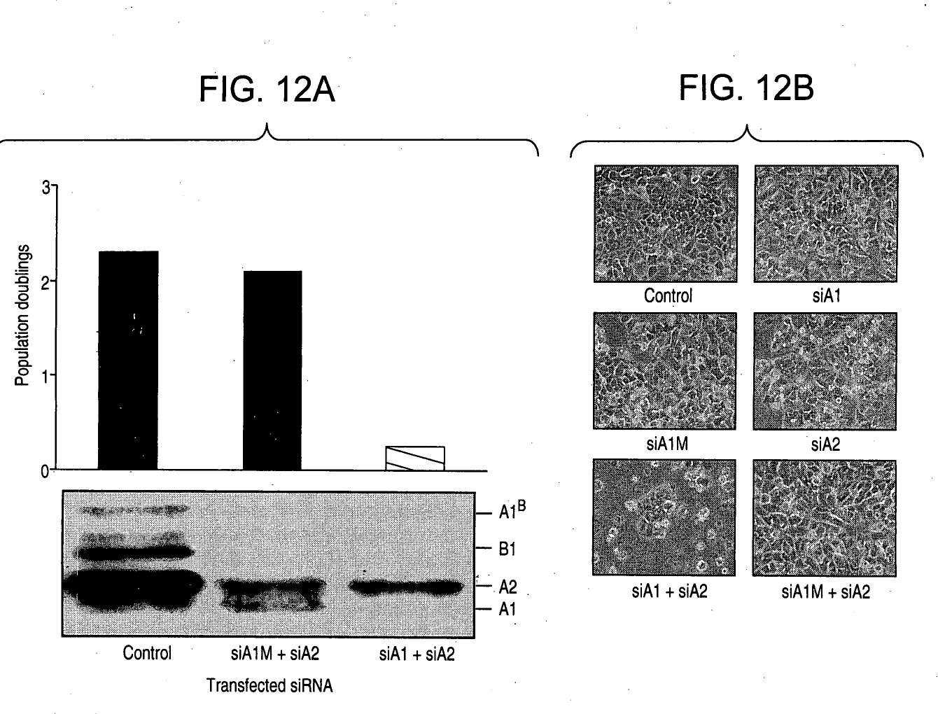

Additional neoplastic cell lines that were tested include the breast carcinoma cell line, MCF-7 (FIG. 12), the ovarian carcinoma cell line, PA-1 and the metastatic ovarian carcinoma SK-OV-3 (ATCC catalog number HTB-77), and the glioblastoma cell line, U373 (see for example, Kanzawa, Cancer Res. 63: 2103-2108, 2003). In all cases, treatment with the hnRNP A1 and A2 combination siRNA pair, A1-1/A2-1, elicited a marked reduction in the expression of hnRNP A1 and A2 polypeptides that was accompanied by a reduction in cell growth and a phenotypic change characteristic of apoptosis. Treatment with individual siRNAs or with the siRNA mismatch control combination (A1-1M/A2-1) displayed no phenotypic changes even when they produced a reduction in hnRNP A1 or A2 expression.

### Example 8

### Reduced Expression of hnRNP A1 and hnRNP A2 does not Affect the Growth of Non-Neoplastic Cell Lines

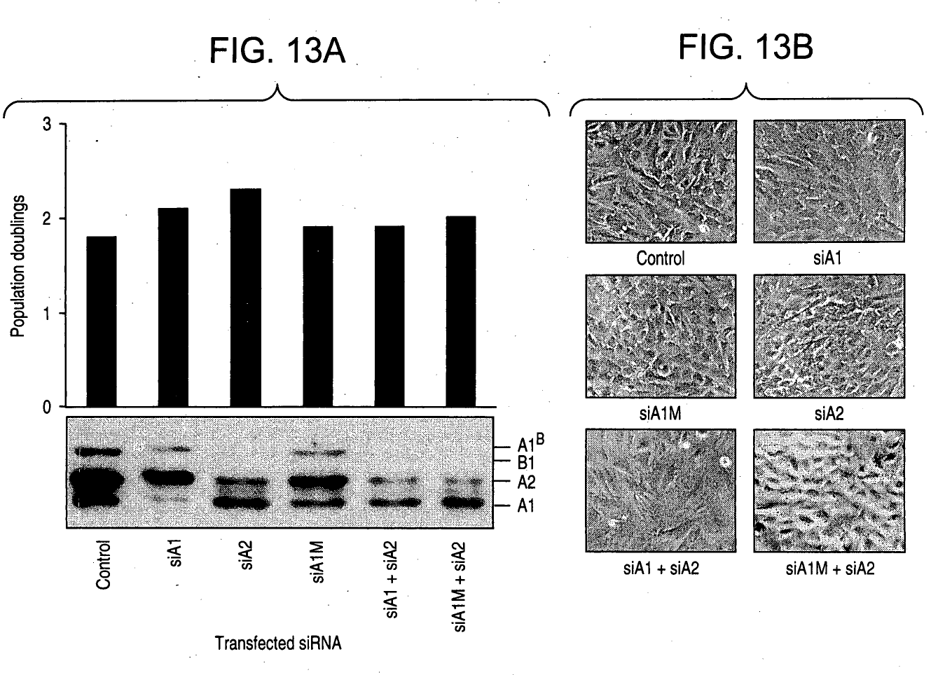

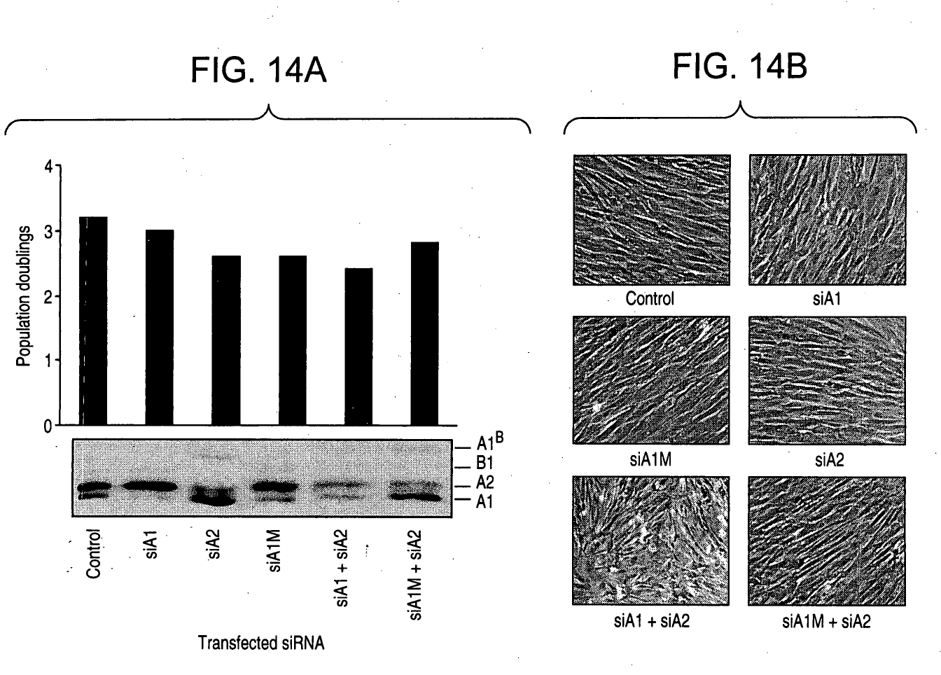

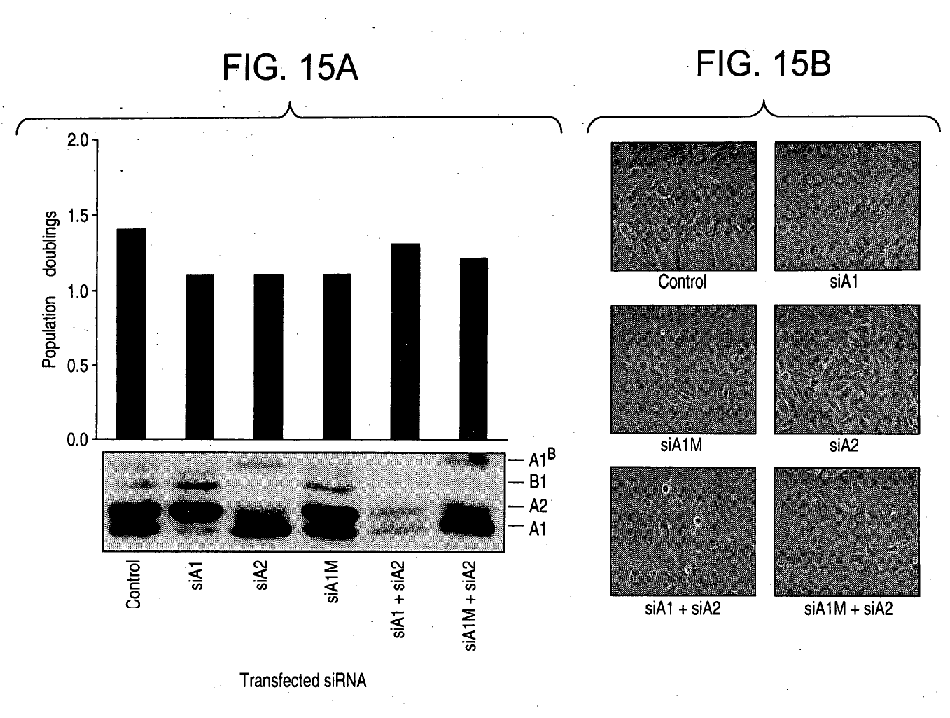

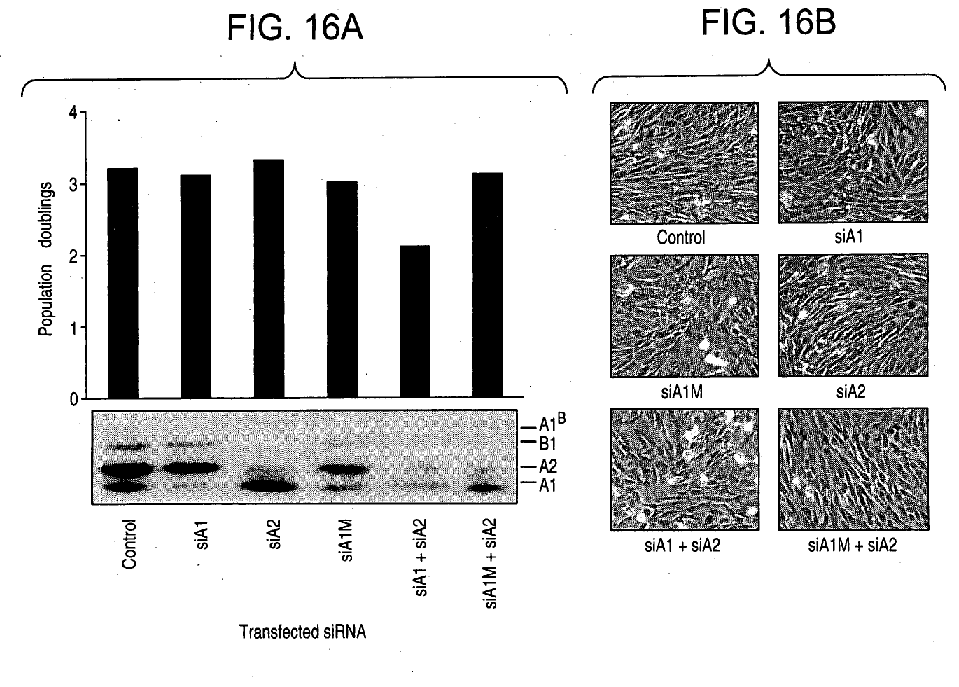

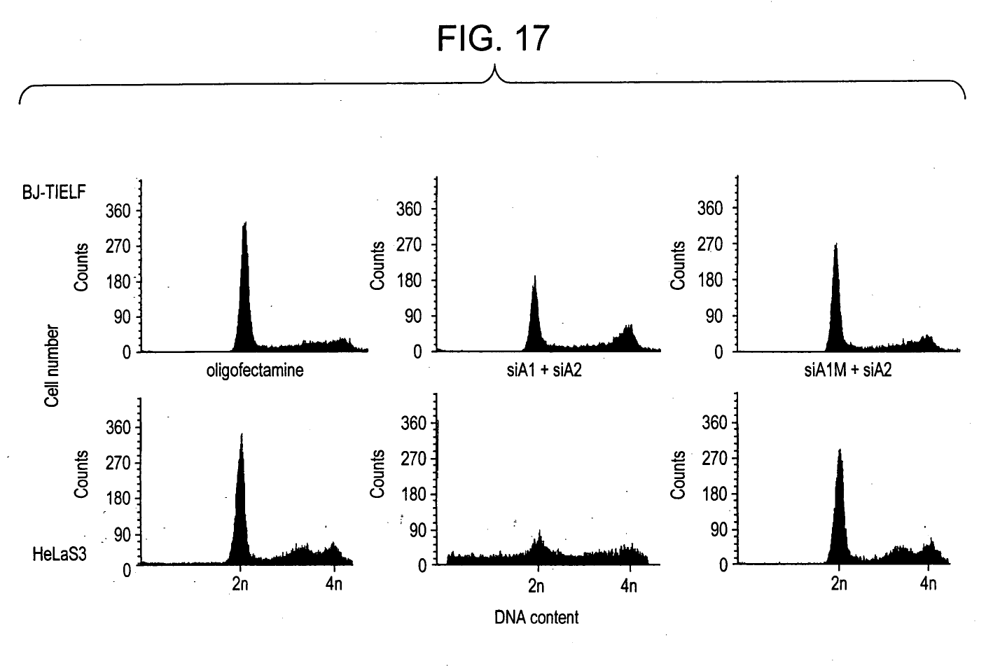

To evaluate the impact of treatment with siRNAs that target hnRNP A1 and hnRNP A2 expression in normal cells, we used three mortal cell lines: colonic myofibroblasts CCD-18Co (FIGS. 13A and 13B), foreskin fibroblasts BJ (FIGS. 14A and 14B), and the epithelial intestinal cell line HIEC (FIGS. 15A and 15B; for cell line see for example, Ruemmele et al., Gut. 51: 842-8, 2002). We also used the BJ-TIELF cell line (FIGS. 16 and 17) that is immortalized, but is an otherwise apparently normal version of the BJ line, expressing the catalytic component (hTERT) of human telomerase (see Patry et al., Cancer Res. 63: 7679-7688, 2003).

Cells were seeded in 6-well were transfected twice with the indicated siRNA alone (80 nM) or with combinations of siRNA (40 nM each siRNA for a total concentration of 80 nM). Control cells were treated with oligofectamine in the absence of siRNA. Trypan blue dye exclusion assays for cell viability were performed 72 hours after the first transfection and cell growth was evaluated (expressed in population doublings). Western analysis was carried out with the polyclonal antibody against A1/A2/A1^{B}/B1. Ponceau S-staining of the nitrocellulose membrane was used to confirm equal protein loading of all lanes (not shown). At 96 hours post-transfection, adherent cells were photographed and both adherent and floating cells were harvested and counted.

Cell viability was evaluated by trypan blue dye exclusion (FIG. 16A) and morphology was evaluated using phase contrast microscopy (200× magnification) (FIG. 16B). DNA content analysis of BJ-TIELF cells treated with siRNA against hnRNP A1 and hnRNP A2 was carried out (FIG. 17). The 96 hour-post transfection profile is compared with a parallel treatment of HeLaS3 cells.

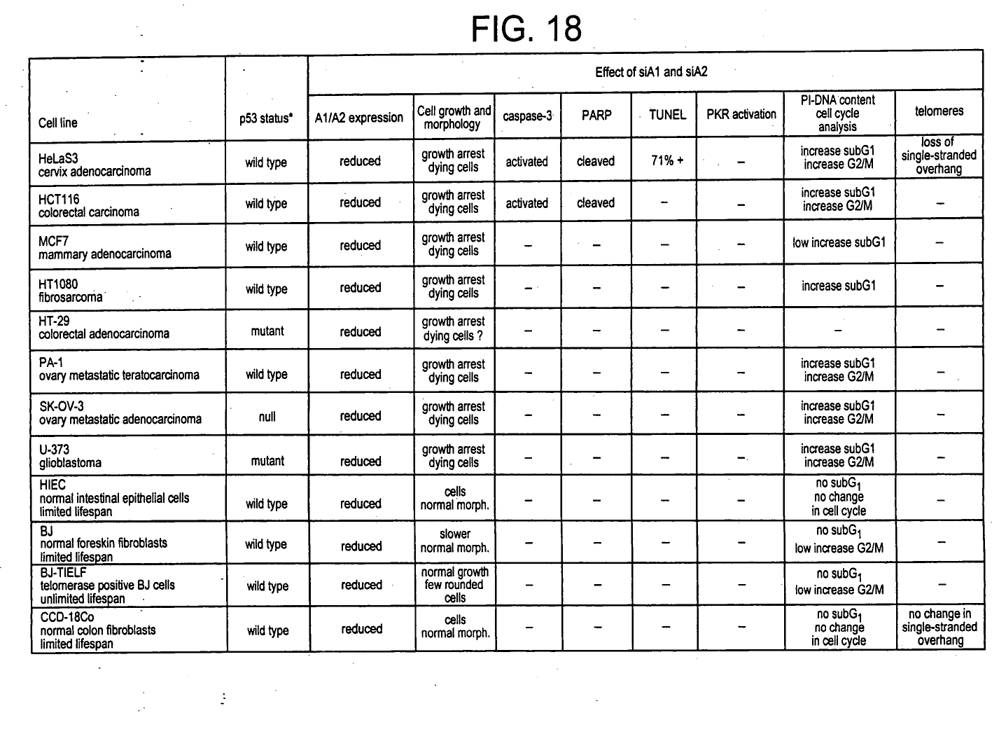

All these mortal cells express hnRNP A1 and A2 proteins (FIG. 18). As noted previously, hnRNP A1 and A2 expression drops when mortal cells approach senescence (Hubbard et al., Exp Cell Res. 218: 241-247, 1995). The immortal BJ-TIELF cell line consistently expressed higher levels of hnRNP A1 and hnRNP A2 proteins than was observed even in early passages of BJ cells.

RNA interference assays with siRNAs targeting hnRNP A1, A2, or both reduced the corresponding protein level. This decrease was comparable to the decrease observed in similarly treated neoplastic cell lines (FIG. 18). In contrast to our results with neoplastic cell lines, described above, the mortal cell lines tolerated a reduction in hnRNP A1 and hnRNP A2 expression, but no significant effects on cell growth and morphology were observed. Even the growth of immortal, but non-transformed, BJ-TIELF cells was not affected by siRNA treatment that decreased hnRNP A1 and A2 expression levels by 50% of the level observed in untreated cells. In all cases examined, cell cycle analysis of the DNA content indicated no subG1 increases. We concluded that mortal human cell lines tolerate a reduction in hnRNP A1 and A2 proteins imposed by RNA interference in contrast to neoplastic cell lines well, with no apparent adverse effects.

### Example 9

### A1 and A2 RNAi Effects on Cell Growth and Protein Expression in Human Cell Lines

A number of normal and cancerous human cell lines were treated with siRNA targeting either hnRNP A1 or hnRNP A2 alone (A1#1, A1#1M, A2#1) or with siRNA combinations targeting both hnRNP A1 and hnRNP A2 (A1#1 and A2#1) or with an A1 mismatch control in combination with an siRNA targeting A2 (A1#1M and A2#1). In each case cells were transfected once at 24 hours and once at 48 hours, and cells were harvested at a timepoint following transfection that allowed for at least 3 to 4 population doublings following the first transfection. Cell growth was measured and protein expression ascertained as described herein. The proportion of apoptotic cells was measured using standard assays. A reduction in hnRNP A1 and A2 expression resulted in extensive cell death in all human neoplastic cell lines tested, independent of their p53 status. Interestingly, in all the normal human cell lines tested, the reduction in A1 and A2 expression never resulted in massive induction of cell death, although in some normal cell lines the siRNA combination that targeted both hnRNP A1 and hnRNP A2 (A1#1 and A2#1) resulted in a slight reduction in cell growth rate and in slight morphological changes.

### Example 10

### hnRNP A1 and hnRNP A2 Expression in Cancer and Normal Tissues

We used rabbit polyclonal antibodies to investigate the expression of hnRNP A1 and A2 in various human cancer biopsies and normal cell types. Immunohistochemistry was performed with an anti-A1 antibody that binds the A1 and A1B proteins, and with an anti-A1/A2 antibody that binds A1/A1^{B}/A2/B1 proteins.

Table I shows hnRNP A1 and hnRNP A2 expression in cancer tissues. The cancer screen was performed on 8 different human cancer types. Three different biopsies per cancer type were analyzed using the rabbit polyclonal anti-A1 and anti A1/A2 sera. The overall result of the nuclear expression of hnRNA A1 and A2 is reported with a note in superscript (^{C}) indicating the status of hnRNP A1 and hnRNP A2 expression in the cytoplasm. Expression levels are reported as follows: Strong: +++, Moderate: ++, Low: +, Very low: +/−, Negative: −.

TABLE I

| | | A1/A2 |
|---|---|---|
| Tumor | Sample | expression¹ |
| Breast cancer | 1 | +++^{c++} |
| | 2 | +++^{c++} |
| | 3 | +++ |
| Colon carcinoma | 1 | +++^{c+++} |
| | 2 | +^{c+++} |
| | 3 | +++^{c++} |
| Lung adenocarcinoma | 1 | +/−^{c+} |
| | 2 | ++^{c++} |
| | 3 | +++ |
| Small Cell Lung carcimoma | 1 | ++^{c++} |
| | 2 | ++^{c++} |
| | 3 | ++ |
| Ovary carcinoma | 1 | ++^{c+} |
| | 2 | ++ |
| | 3 | +++^{c++} |
| Pancreas carcinoma | 1 | +/−^{c+++} |
| | 2 | +++^{c+/-} |
| | 3 | ++^{c++} |
| Prostate carcinoma | 1 | ++^{c++} |
| | 2 | −^{c++} |
| | 3 | ++ |
| Skin melanoma | 1 | ++ |
| | 2 | ++^{c++} |
| | 3 | +/− |

¹Expression levels: Strong: +++, Moderate: ++, Low: +, Very low: +/−, Negative: −.

Table II shows hnRNP A1 and hnRNP A2 expression in normal tissues. The normal tissue screen was performed on 10 different normal human tissues (one sample per tissue) using both an the anti-A1 and an the anti-A1/A2 sera. Two different sections of the same tissue sample were independently treated with each serum. Results are given for the cell types that were observed in each section. The overall results of the nuclear expression of hnRNP A1 and hnRNP A2 is reported with a note in superscript (^{C}) indicating the status of hnRNP A1 and hnRNP A2 expression in the cytoplasm.

TABLE II

| | | A1/A2 |
|---|---|---|
| Tissue | Cell type | expression¹ |
| Brain | neurons (some) | ++ |
| | neutrophils | −^{c+/-} |
| | astrocytes, microglia, oligodendrocytes, | − |
| | endothelium, vascular smooth muscle | |
| Heart | cardiac myocytes, endothelial cells, vascular | − |
| | smooth muscle, fibroblasts | |
| Kidney | endothelium, thick and thin loop of Henle, | +/++ |
| | glomerular capillary and collecting duct | |
| | endothelium, vascular smooth muscle | |
| | Bowman's capsule epithelium, podocytes, | +/++ |
| | proximal and distal convoluted tubules | |
| | mesanglial cells | − |
| Liver | hepatocytes, endothelium, lymphocytes, | + |
| | vascular smooth muscle | |
| | bile duct | ++ |
| | fibroblasts | − |
| | macrophages, Kupffer cells | −^{c+} |
| Lungs | pneumocytes, fibroblasts, endothelium, | + |
| | mesothelium | |
| | alveolar macrophages | −^{c++} |
| Pancreas | endothelium, vascular smooth muscle, | − |
| | fibroblasts, adipocytes | |
| | peripheral islets cells | −^{c+++} |
| | acinar epithelium | +/−^{c++} |
| | pancreatic duct | +/− |
| Skeletal | myocytes | +^{c++} |
| muscle | vascular smooth muscle | −^{c+/-} |
| | endothelium | + |
| | fibroblasts | − |
| Skin | squamous epiuthelium (basal layer) | ++/+++ |
| | squamous epithelium (nucleated layer), | + |
| | superficial dermal fibroblasts, endothelium, | |
| | lymphocytes | |
| | stratum lucidum, eccrine sweet glands | −^{c+/-} |
| | subcutaneous glands | − |
| | vascular smooth muscle | −^{c++} |
| | mast cells | −^{c+} |
| Small | neuroendocrine cells, epithelium (bases of | −^{c+} |
| Intestine | crypts) | |
| | villi columnar epithelium, lymphocytes | + |
| | goblet cells, Schwann cells | − |
| | macrophages | ++^{c+} |
| | smooth muscle | +/−^{c++} |
| | fibroblasts, ganglion cells, endothelium | +/− |
| Spleen | smooth muscle, macrophages | −^{c+} |
| | lymphocytes, mesothelium | +/++ |
| | fibroblasts | − |
| | neutrophils | + |
| | endothelial cells | −^{c++} |

¹Expression levels are reported as follows: Strong: +++, Moderate: ++, Low: +, Very low: +/−, Negative: −.

Most normal tissues examined expressed low or undetectable levels of hnRNP A1 and hnRNP A2 proteins, except for the basal layer of the skin, which expressed high levels of A1. Low, or occasional, A1 expression was observed in some neurons, kidney epithelia and endothelium, liver Kuppfer cells, macrophages, bile duct, neuroendocrine tissue, macrophages, crypt cells of the small intestine, lymphocytes, and mesothelium of the spleen.

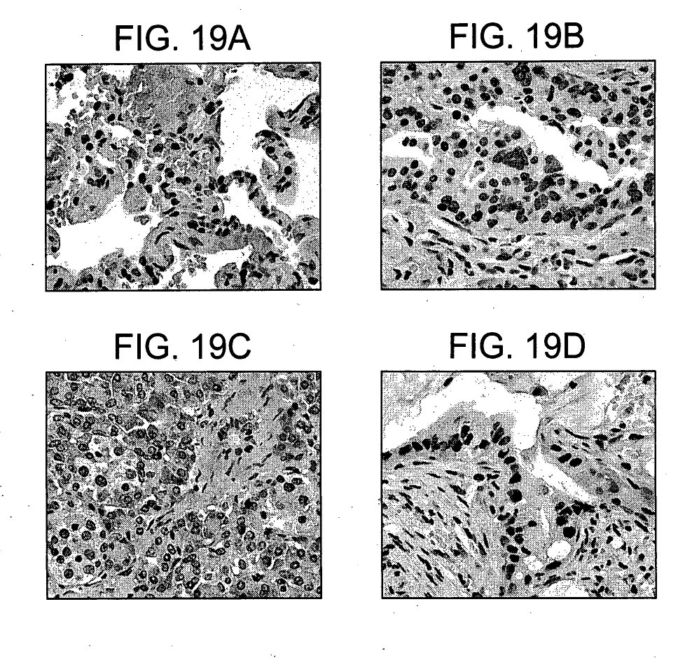

Higher expression of hnRNP A1 and hnRNP A2 proteins was observed in tumor cells relative to normal cells (Table II and FIG. 19). This expression profile identifies A1 and A2 as a useful markers for cancer detection. The functional association that links hnRNP A1 with telomere biogenesis suggests that A1 plays a crucial role in maintaining the transformed state of neoplastic cells, possibly via its role as a telomeric capping factor. Several reports have documented a high level of expression of A2, and its spliced isoform B1, in lung cancer (Zhou et al., J. Biol. Chem. 271: 10760-10766, 1996; Sueoka et al., Cancer Res. 59: 1404-1407, 1999).

Recent studies have also identified A2/B1 as early markers for pancreatic and breast cancers (Yan-Sanders et al., Cancer Lett., 183: 215-220, 2002; Zhou et al., Breast Cancer Res. Treat. 66: 217-224, 2001). Given the amino acid sequence identity between A1 and A2, and the fact that both bind telomeric repeats in vitro, it appeared that these proteins are functional homologues. Consistent with this view, hnRNP A1 and hnRNP A2 control in vitro alternative pre-mRNA splicing in a very similar manner (Hutchison et al., J. Biol. Chem. 277: 29745-52, 2002). Distinct sets of multiple heterogenous nuclear ribonucleoprotein (hnRNP) A1 binding sites control 5′ splice site selection in the hnRNP A1 pre-mRNA.

### Example 10

### hnRNP A1 and hnRNP A2 Expression in Cancerous and Benign Tissues

FIGS. 19A and 19B show an immunohistological analysis using anti-hnRNP A1 or anti-hnRNP A1/A2 antiserum in benign and cancerous breast (FIG. 19A) and pancreatic tissues (FIG. 19B).

### Example 11

### RNAi on hnRNP A1 and A2 in Mouse Cells

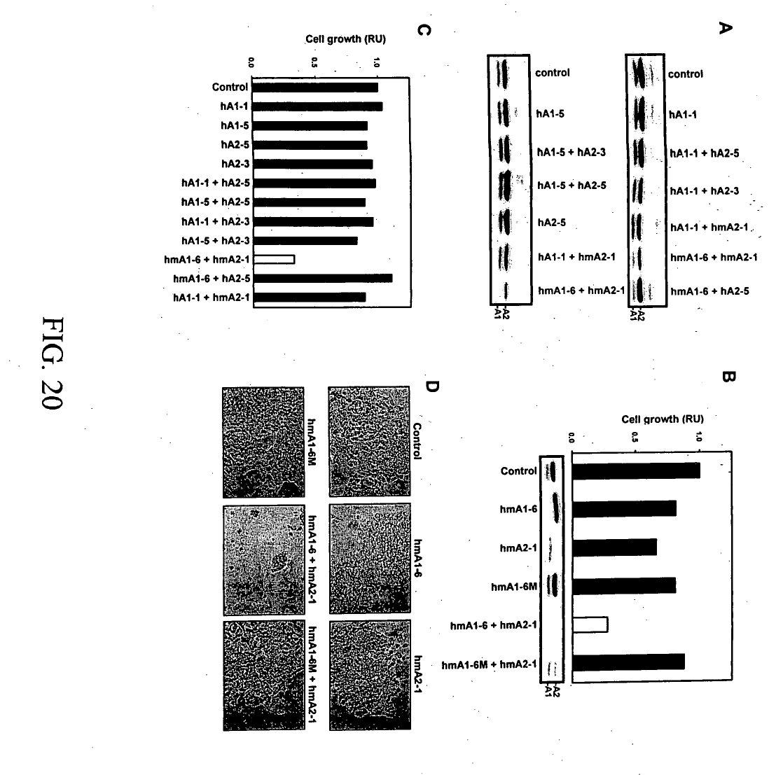

To determine if hnRNP A1 and/or hnRNP A2 expression in mouse cells is essential for cell growth, we used siRNAs to reduce the levels of A1 and/or A2 proteins in mouse neoplastic cell lines. Double-stranded siRNAs against A1 or A2 were introduced into the testicular embryonic carcinoma F9 cell line by performing two successive applications of siRNAs at a 24 hour interval. Control samples were treated with Lipofectamine 2000 in the absence of siRNA. siRNAs hmA1-6 and hmA2-1, which have been shown to be active against human A1 and A2, respectively, also match the sequence of the mouse A1 and A2 cDNAs (see Table III). The combined application of siRNAs hmA1-6 and hmA2-1 promoted a marked reduction in the expression levels of both A1 and A2 proteins in the mouse cells (FIGS. 20A and 20B). In contrast, a set of human-specific siRNAs which contain mismatches when compared to mouse transcripts (hA1-1, hA1-5 and hA2-5) did not elicit a change in the abundance of the mouse A1 or A2 proteins (Table III, FIGS. 20A and 20B). As expected, the mutated siRNA hmA1-6M also did not elicit a reduction in the level of mouse A1 (FIG. 20B). Although the human-specific siRNA hA2-3 harbors one mismatch with the mouse A2 transcript (Table III), it displayed activity in F9 cells on several occasions. This positive result is not unexpected because the mismatched position would create a G•U base-pair with the A2 mRNA and therefore may still permit RNA interference activity.

TABLE III
Activity of siRNA

| | | |
|---|---|---|
| SEQUENCE | A1/A2 mRNA Target | siRNA Activity |

| | | | | | |
|---|---|---|---|---|---|
| siRNA | (AA-N19) mRNA target | Human | Mouse | Human ^{a} | Mouse ^{b} |

| | | | | | |
|---|---|---|---|---|---|
| hA1-1 | 5′-UGG GGA ACG CUC ACG GAC UdTdT-3′ | Yes | No | Yes | No |
| hA1-1M | 5′-UGG GGA ACC GUC ACG GAC UdTdT-3′ | No | No | No | No |
| hmA1-2 | 5′-UGA GAG AUC CAA ACA CCA AdTdT-3′ | Yes | Yes | Yes | Yes |
| hA1-5 | 5′-AUC AUG ACU GAC CGA GGC AdTdT-3′ | Yes | No | Yes | No |
| hmA1-6 | 5′-CUU UGG UGG UGG UCG UGG AdTdT-3′ | Yes | Yes | Yes | Yes |
| hmA1-6M | 5′-CUU UGG UGU GGG UCG UGG AdTdT-3′ | No | No | No | No |
| hmA2-1 | 5′-GCU UUG AAA CCA CAG AAG AdTdT-3′ | Yes | Yes | Yes | Yes |
| hmA2-2 | 5′-CCA CAG AAG AAA GUU UGA GdTdT-3′ | Yes | Yes | Yes | Yes |
| hA2-3 | 5′-GAA GCU GUU UGU UGG CGG AdTdT-3′ | Yes | No | Yes | Yes ^{c} |
| hA2-5 | 5′-CUU UGG UGG UAG CAG GAA CdTdT-3′ | Yes | No | Yes | No |

^{a} HeLaS3 cells,
^{b} F9 cells,
^{c} occasional activity

To assess whether the siRNA treatments affected the growth of F9 cells, we counted the total number of cells 72-hours following the first treatment with various siRNA mixtures (FIGS. 20B and 20C). Cellular morphology was also assessed by microscopic examination (FIG. 20D). Individual or combined siRNA treatments with human-specific siRNAs did not affect cell growth (FIG. 20C). Likewise, mixtures that contained siRNAs targeting the mouse A1 or the A2 transcripts separately did not impact cell growth (FIGS. 20B and 20C). In contrast, the mixture of siRNAs that targeted both mouse A1 and A2 transcripts promoted a considerable drop in cell growth and F9 cells displayed an altered morphology (hmA1-6/hmA2-1; FIGS. 20B, 20C, and 20D).

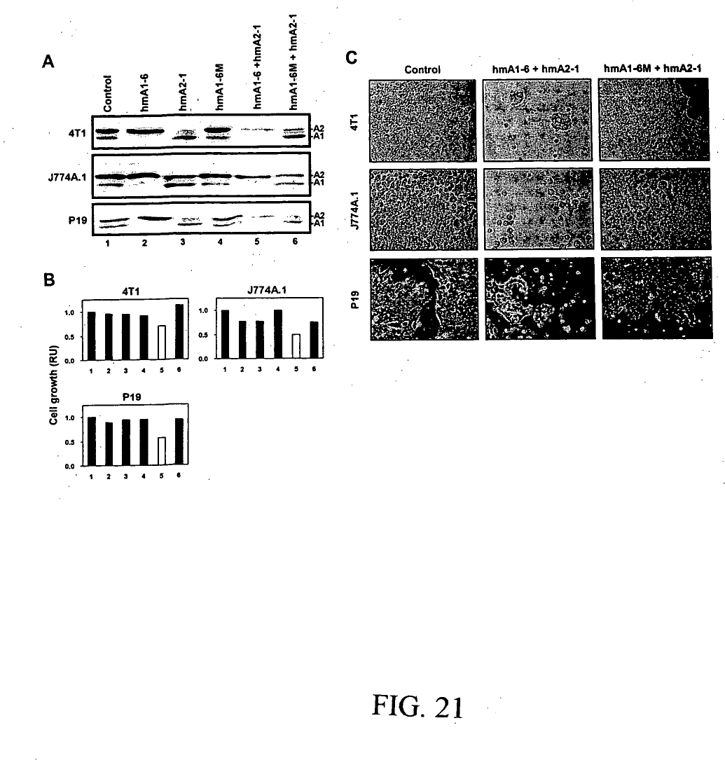

The ability of RNAi to reduce A1 and A2 levels and impede cell growth was also investigated in cell lines derived from other mouse cancers. We tested the mammary metastatic neoplastic cell line 4T1, the macrophage sarcoma cell line J774A.1 and the teratocarcinoma P19 cell line. Cell viability was measured 72 hours post-transfection as before. Similar to what was observed in F9 cells, treatment with individual siRNAs promoted a reduction in the targeted protein(s) (FIG. 21A), but only the combination of siRNAs targeting both mouse A1 and A2 affected the growth of 4T1, J777A.1 and P 19 cells (hmA1-6/hmA2-1; FIGS. 21B and 21C). The mutated hmA1-6 (hmA1-6M) alone or in combination with hmA2-1 did not elicit a reduction in A1, and the cells displayed normal growth and morphology.

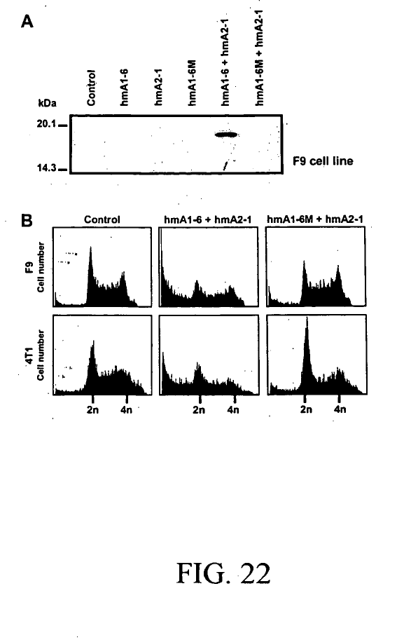

A trypan blue exclusion staining of treated cells indicated that the siRNA mixture that affected cell growth and morphology promoted cell death. To assess whether cell death was occurring by apoptosis, we carried out a procaspase-3 cleavage assay as well as DNA content analysis (FIGS. 22A and 22B). Only the treatment with the pair of active siRNA (hmA1-6/hmA2-1) promoted the detection of the procaspase-3 cleavage product in the F9 cell line (FIG. 22A). The DNA content analysis performed on F9 and 4T1 cells showed the characteristic sub-G1 increase expected for DNA fragmentation (FIG. 22B). These results suggest that the rapid cell death of mouse cells induced by siRNAs against A1/A2 occurs by apoptosis.

### Example 12

### Non-Transformed Mouse Cell Lines are Resistant to the RNAi-Mediated Decrease in A1/A2

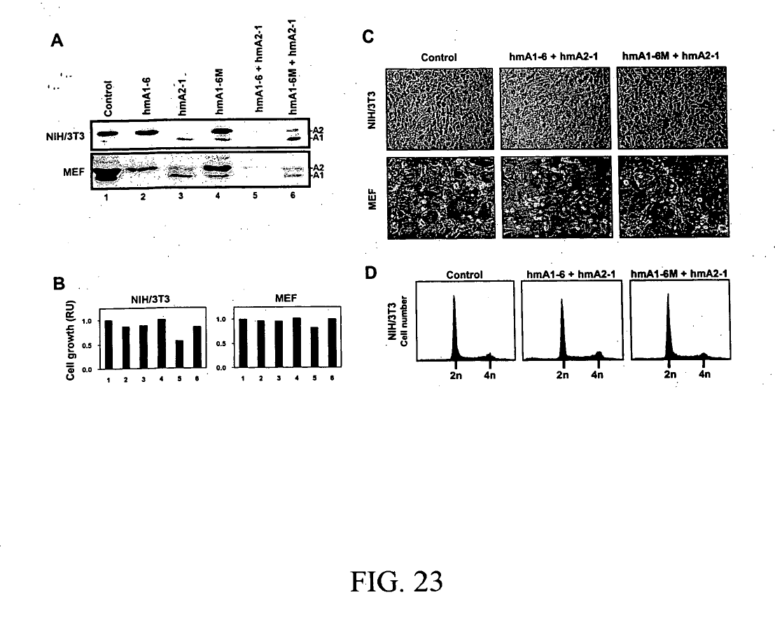

To monitor the impact of a reduction in A1/A2 levels on normal mouse cells, we used two cell lines: NIH3T3 fibroblasts and mortal mouse embryonic fibroblasts (MEF). MEF cells were obtained from 13-15 day old embryos of BALB/c mice. NIH3T3 and MEF cells express A1 and A2 proteins at levels that are comparable to the levels detected in the mouse transformed cell lines used above. RNAi assays with siRNAs against A1, A2, or both, promoted a reduction in the corresponding proteins that was equivalent to the reduction observed in similarly treated transformed cells (FIG. 23A). In contrast to neoplastic cells however, the treatment was well tolerated by both NIH3T3 and MEF cells, and had little effect on cell growth (FIG. 23B) or cell morphology (FIG. 23C). Moreover, abrogation of A1/A2 expression in NIH3T3 cells was not accompanied by an increase in the sub-G1 peak of cells (FIG. 23D).

### Example 13

### Protective Effect of Mouse A1 Expression on RNAi-Treated Human Cells

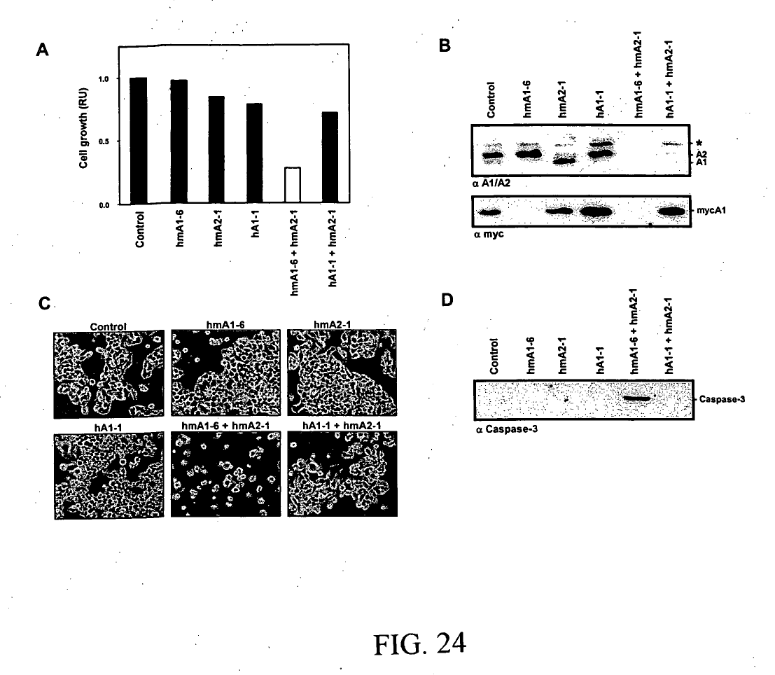

The mouse results paralleled and corroborated results previously obtained with human cells. Taken together, the data suggest a homologous essential function of mouse and human A1/A2 in transformed cells. Although highly homologous in sequence, mouse and human A1 mRNAs do differ at various positions and siRNAs completely complementary only to human, mouse or both can be derived (Table III). Such common or species-specific siRNAs therefore provided the necessary tools to examine the specificity of the siRNA treatments. To assess the cell death phenotypes induced by siRNAs against hA1/hA2 in human cells can be rescued by expressing the mouse A1 protein, we carried out siRNA treatments of HeLa S3 cells engineered to express the mouse A1 cDNA. HeLa S3 cells were transfected with a plasmid expressing a myc-tagged mouse A1 cDNA and a clonal derivative was isolated. Expression of the myc-tagged mouse A1 was confirmed by performing a western blot with anti-myc antibody (FIG. 24B). When this cell line is treated with a mixture of siRNAs that target the mouse and human A1 (siRNA hmA1-6), as well as the human A2 (siRNA hmA2-1), expression of all three proteins (human A1, A2; mouse myc-tagged A1) was markedly decreased (FIG. 24B), cell growth and cell morphology were severely impaired (FIGS. 24A and 24C), and rapid cell death occurred. In contrast, HeLa cells treated with siRNAs that only abrogated human A1 and A2 (hA1-1/hmA2-1) grew almost normally and did not display the phenotypes associated with cell death (FIGS. 24A and 24C). These data demonstrate that expression of the mouse A1 cDNA protects HeLa S3 cells from the deleterious effect of reducing the levels of human A1 and A2 proteins. Therefore, the cell death observed in both the human cells and the mouse cells is caused by the simultaneous reduction in A1 and A2 proteins, and not through some non-specific effects of the active pair of siRNAs or via targeting an unrelated RNA, which would carry a stretch of sequence complementary to the siRNAs. This result confirms that the reduction in A1/A2 is intimately associated with the induction of apoptosis.

Therapeutic Applications

The successful treatment of cancer depends on the identification of therapeutic targets whose expression is restricted to neoplastic cells and which function to promote or permit unlimited cell growth. The ideal target is essential to a broad array of neoplastic cell phenotypes and the ideal therapeutic is one which has no negative effect on the health of the organism being treated. Although varied targets have been identified in different types of cancer, there are very few examples of factors that play a ubiquitous role in virtually all types of cancers. Telomeric factors whose expression is restricted to neoplastic cells represent a major advance towards novel cancer therapeutic strategies because the maintenance of functional telomeres is essential for neoplastic cell division, regardless of the mechanisms leading to the development of a cancer.

In the experiments described herein, we have identified the hnRNP A1 and A2 proteins, and their alternatively spliced isoforms A1^{B} and B1, respectively, as targets for cancer therapeutics. While it was known that hnRNP A1 and A2/B1 proteins were expressed at high levels in colon and lung cancers, respectively, we now show that moderate to high levels of hnRNP A1 proteins are detected in breast, lung, colon, prostate, ovary, pancreas and skin cancers. Levels of hnRNP A1 and hnRNP A2 proteins in normal tissues are generally much lower than that observed in neoplastic cells. Only the basal layer of the skin expressed high levels of hnRNP A1 and A2 proteins.

Remarkably, we find that neoplastic cell lines from many different species and tissue origins were sensitive to decreases in the levels of hnRNP A1 and A2 proteins. The RNAi-mediated reduction in hnRNP A1 and A2 expression levels, as well as A1^{B} and B1 expression levels in some instances, usually elicited the death of neoplastic cells by apoptosis within 96 hours.

A reduction in hnRNP A1 or A2 protein alone did not induce apoptosis, possibly because hnRNP A1 and A2 are functional homologues that can compensate for one another. It is likely that hnRNP A2, which is normally expressed at a slightly higher level in these cells, partially compensated for reductions in A1 function, when A1 alone was targeted, allowing the cells to survive. Thus, in a situation where A1 and A2 are expressed in equimolar amounts, it may be virtually impossible to reduce the global level of hnRNP A1 and hnRNP A2 by 50% by targeting either A1 or A2 alone. Only by targeting A1 and A2 in combination is it possible to achieve a global reduction in both hnRNP A1 and hnRNP A2 levels and to functionally inhibit cell growth. Still, it is possible that in some cell types, hnRNP A1 and A2 expression may be independently controlled. For example, some neoplastic cells may express higher levels of hnRNP A1 and lower levels or no hnRNP A2. In such cell types, targeting either A1 or A2 individually might inhibit cell growth and induce programmed cell death.

The results described above suggest that the reduction in hnRNP A1 and A2 proteins affect a common mechanism in a large variety of different mouse and human neoplastic cells. The rapidity with which neoplastic cells die following treatment with hnRNP A1 and A2-specific siRNAs is consistent with hnRNP A1 and A2 proteins acting as telomeric capping factors. Although not wishing to be bound by theory, there are several factors that support this theory. First, the hnRNP A1 and A2 proteins bind with high affinity to single-stranded telomeric sequences in vitro. Second, both the telomeric repeat sequence TAGGGT and the amino-acid sequence of the hnRNP A1 and A2 proteins are perfectly conserved between mouse and human. Third, a reduction in hnRNP A1 protein alone in mouse cells is associated with telomere shortening and restoring or overexpressing hnRNP A1 in mouse and human neoplastic cells promotes telomere elongation. Fourth, we have now shown that reduced hnRNP A1 and A2 expression is accompanied by a decrease in the length of the telomeric single-stranded G-rich extensions. Because this shortening can be detected between 48 and 72 hours after siRNA treatment, a reduction in the size of telomeric overhangs may be triggering cell growth arrest that would in turn elicit neoplastic cell apoptosis. Most importantly, this decrease in the length of G-rich extensions is not observed when cells are treated with the apoptotic inducer staurosporine, suggesting that the degradation of single-stranded telomeric repeats is not necessary for apoptosis to occur. Fifth, the effects observed are independent of overall telomere tract length. Thus, overall our results are consistent with the view that in both mouse and human transformed cells, hnRNP A1 and A2 are part of the telomeric cap that protects telomeres and prevents them from being recognized as double-stranded breaks. A reduction in hnRNP A1 and A2 may therefore at least partially disrupt the telomeric cap, expose the single-stranded telomeric G-tails to nucleases, ultimately leading to telomere dysfunction and induction of apoptosis. The fact that mouse and human normal cells are resistant to similar reductions in hnRNP A1 and A2 levels suggests that the structure of the telomeric cap in such cells may be different from that of neoplastic cells.

It is also very interesting to note that the apoptosis induction appears independent of the status of p53 expression since p53 null and p53 mutant cell lines were equally sensitive to RNAi against hnRNP A1 and A2. In contrast, apoptosis triggered by a dominant negative mutation in the telomeric factor TRF2 required the presence of wild-type p53 protein. These results suggest that mutated TRF2 and reduced levels of hnRNP A1 and A2 trigger different events that lead to apoptosis.

In sharp contrast, the siRNA-mediated reduction in hnRNP A1 and A2 levels in mortal cell lines did not affect cell division and did not induce cell death. The fact that these “normal” cell lines are resistant to decreases in hnRNP A1 and A2 expression is intriguing and suggests that differences exist in the telomere capping structure of neoplastic cells and normal cells. This is not entirely unexpected given that telomerase is usually expressed in neoplastic cells but is usually not expressed in normal cells. Telomerase expression, and other factors, may lead to differences in the size of the single-stranded G-rich extensions, possibly affecting the identity and function of capping factors. The hPot1 protein has recently been shown to associate with human telomeric single-stranded extensions. Future studies should clarify the expression profile of hPot1 and its contribution to the telomere capping function in normal and neoplastic cells.

In summary, we have demonstrated that decreases in both hnRNP A1 and A2 caused apoptosis in a variety of mouse and human neoplastic cell lines, including p53-compromised cells and that this apoptosis was specific to cancer cells. Our findings establish hnRNP A1 and hnRNP A2 as excellent drug targets in cancer therapeutics and have allowed us to design therapeutics that abrogate the expression and/or function of hnRNP A1 and hnRNP A2 proteins in neoplastic cells. Furthermore, the identical response to RNAi-mediated reduction of A1/A2 protein levels displayed by human and mouse neoplastic cell lines indicates that the mouse is an excellent model organism for testing potential anti-cancer compounds targeting hnRNP A1 and A2. Such approaches are particularly attractive given that hnRNP A1 and hnRNP A2 are expressed at low levels in normal tissues, and that reducing hnRNP A1 and hnRNP A2 levels in mortal cell lines does not significantly affect their cell growth or survival.

Methods

Cell Culture

HeLaS3, HCT 116, HT-1080, MCF-7 and CCD-18Co cells were from the American Type Culture Collection. BJ foreskin normal fibroblasts were kindly provided by James Smith (Baylor College of Medicine, Houston). HIEC cells were from Jean-Francois Beaulieu (Université de Sherbrooke, Québec). PA-1 and SK-OV-3 cells were provided by Claudine Rancourt (Université de Sherbrooke, Québec). U387 were kindly supplied by David Fortin (Université de Sherbrooke, Québec). HeLaS3 and U-373 MG cells were grown in DMEM supplemented with 10% FBS. HCT 116 cells were grown in McCoy's 5A media supplemented with 10% FBS. BJ and BJ-TIELF cells were grown in αMEM supplemented with 10% FBS. HIEC cells were grown in Opti-MEM I supplemented with 5% FBS. PA-1 and SK-OV-3 cells were grown in DMEM-F12 supplemented with 10% FBS. MCF-7 cells were grown in EMEM supplemented with 10% FBS, 0.1 mM non-essential amino acids and 10 μg/ml bovine insulin. HT-1080 and CCD-18Co cells were grown in αMEM supplemented with 10% FBS, Earle's salt, 1 mM sodium pyruvate, 0.1 mM non-essential amino acids.

4T1, F9, P19, J774A.1 and NIH/3T3 cells were obtained from the American Type Culture Collection. MEF were prepared from 13-15 days old embryos of BALB/c mouse obtained from Charles River Laboratories. F9 cells were grown on gelatin-coated dishes in DMEM supplemented with 10% FBS. 4T1 cells were grown in RPMI 1640 supplemented with 10% FBS. P19 cells were grown in alpha-MEM supplemented with 10% FBS. J774A.1, NIH/3T3, and MEF cells were grown in DMEM supplemented with 10% FBS.

Transfection

The day before transfection, exponentially growing cells were trypsinized, counted, and seeded in 6-well plates so that they were 30-50% confluent on the day of transfection. Transfections were performed on cells using Oligofectamine (Invitrogen) or Lipofectamine 2000 (Invitrogen), according to manufacturer's instructions. Concentrations of 80 nM and 40 nM of total siRNAs were used for mouse cell lines and the HeLa S3 clone, respectively.

See below for the appropriate number of cells and media for specific cell lines; note that some cell lines need FBS whereas for other cell lines it is important not to add FBS. Antibiotics were avoided at the time of plating and during transfection; cell cultures below 20 passages were always selected.

| Cell line | Cell number/well | Culture media |
|---|---|---|
| HeLa S3 | 65,000 | DMEM + 10% FBS |
| HCT116 | 65,000 | McCoys5A + 10% FBS |
| HT-29 | 50,000 | McCoys5A + 10% FBS |
| MCF7 | 100,000 | MEM Earle's Salt w/o FBS |
| HT1080 | 50,000 | MEM Earle's Salt w/o FBS |
| HIEC | 100,000 | OPTI-MEM I + 5% FBS |
| BJ | 100,000 | α-MEM w/o FBS |
| BJ-TIELF | 50,000 | α-MEM w/o FBS |
| 18Co | 100,000 | MEM Earle's Salt w/o FBS |

Cells were incubated overnight at 37° C./5% CO₂. On the day of transfection, mix #1 was prepared for each well and incubated at room temperature for 5 to 10 minutes:

Mix #1

10 μl of siRNA (8 μM stock prepared by diluting the 50 μM stock)+175 μl OPTI-MEM I (Invitrogen Cat. #51985-034).

Transfection reagent for each well (mix #2) was also prepared:

Mix #2

4 μl Oligofectamine (Invitrogen Cat. #12252-011)+11 μl OPTI-MEM I.

Mix #2 was added to mix #1, mixed gently, and incubated at room temperature for 20 minutes. The culture media was removed and 800 μl of fresh media was added to each well (use the same media as for the overnight culture). The complex was mixed and overlayed onto the cells. The final concentration of siRNA was 80 nM. The cells were incubated with the mixed compound for 4 h at 37° C./5% CO₂. 1.0 ml of growth media containing 2 times the normal concentration of serum was added without removal of the transfection mixture. The cells were incubated at 37° C./5% CO₂. A second, identical transfection was performed 24 h after the first one.

Cell viability, cell growth and protein expression were assayed 48-144 hours after the first transfection. Depending on the cell line and the analysis, the incubation time varied as described below.

| | Incubation time before | | Incubation time before | |
|---|---|---|---|---|
| Cell line | protein expression assay | | cell viability assay | |
| HeLa S3 | 72-144 | h | 72-144 | h |
| HCT116 | 48-72 | h | 72 | h |
| HT-29 | 48-96 | h | 96 | h |
| MCF7 | 96 | h | 96 | h |
| HT1080 | 96 | h | 96 | h |
| HIEC | 96-168 | h | 96-168 | h |
| BJ | 96-168 | h | 96-168 | h |
| BJ-TIELF | 96-168 | h | 96-168 | h |
| 18Co | 72-168 | h | 72-168 | h |

Measurement of Cell Viability by Trypan Blue Dye Exclusion Assay

For each well of transfected cells, the culture media was transferred into a 2.0 ml microfuge. Cells were centrifuged (quick spin) to recover the cells that were in suspension and the supernatant was discarded. The adherent cells of each well were rinsed with 400 μl of PBS/EDTA (170 mM NaCl, 3.3 mM KCl, 10 mM Na₂HPO₄, 1.8 mM KH₂PO₄, 0.5 mM EDTA, 0.0015% phenol red). PBS/EDTA was transfered to the 2.0 ml micro tube containing the corresponding pellet of floating cells. 300 μl of 0.06% trypsin in PBS/EDTA was added and incubated for 5 minutes. The trypsinized cells were recovered and transferred to the corresponding 2.0 ml microtube. Each well was rinsed with 400 μl PBS/EDTA, and transferred to the cell suspension. Final volume was determined 50 μl of cell suspension was mixed with 50 μl of trypan blue stain. The trypan blue mix was loaded into the chamber of a hemacytometer and the living (unstained) and dead (blue) cells were counted. The number of cells contained in the total recovered volume was determined. The suspensions were combined, centrifuged for 1 min. and the supernatant was discarded. Cell pellets were resuspended in 100 μl Laemmli Buffer, sonicated to reduce viscosity, and incubated for 3 min in a boiling water bath. Protein concentration was measured on a 10 μl aliquot by the method of Lowry. Samples were stored at −20° C. until they were used for Western blot analysis (15 to 25 μg/lane on SDS-PAGE) of protein expression.

Measurement of Cell Growth

Cell growth was measured by calculating the number of population doublings since transfection using the equation: PD=log (N_{f}/N₀)/log2 where:

PD: number of population doublings

N_{f}: Final number of cells (living and dead cells as counted after trypan blue exclusion).

N₀: number of cells at the time of transfection (average number of 110,000 for HeLaS3 cells; 150,000 for HCT116 cells; 80,000 for MCF7 cells).

Cell growth values for siRNA-treated cells were normalized relative to control treatment and were a mean of at least three independent experiments.

Anti-hnRNP Antibodies

Rabbit polyclonal sera raised against either a peptide unique to the hnRNP A1 protein peptide sequence: (ASASSSQRGR) or against a peptide common to both hnRNP A1 and A2 proteins (KEDTEEHHLRDYFE) was used to carry out the immunohistochemical studies. Peptide synthesis and antibody production was carried out by the Eastern Quebec Proteomic Center (Quebec City). The specificity of each serum was confirmed by ELISA and western analyses.

Immunohistochemistry

The normal tissue screen was performed on 10 different normal human tissues (brain, heart, kidney, liver, lung, pancreas, skeletal muscle, skin, small intestine and spleen) using both sera. Two different sections of the same tissue sample were independently treated with each serum. The cancer screen was performed on 8 different human cancer types (breast carcinoma, colon carcinoma, lung adenocarcinoma, lung small cell carcinoma, ovary carcinoma, pancreas carcinoma, prostate carcinoma and skin melanoma). Three different samples per cancer type were screened using the anti-A1 and the anti-A1/A2 sera. Immunohistochemistry was conducted by LifeSpan BioSciences Inc. (Seattle, Wash.).

siRNAs

Oligonucleotides were purchased from Dharmacon Research, Inc. (Lafayette, Colo.). The nucleic acid sequences to be targeted were identified as follows. The mRNA sequence to be targeted was BLAST searched against the human genome to ensure that only one human gene was targeted by each siRNA. Seven siRNAs targeting the human hnRNP A1 mRNA (GenBank accession number NM_{—}002136) were tested. They covered nucleotides 107 to 127 from the start codon (A1-1), 135 to 155 (A1-2), 154 to 174 (A1-3), 217 to 237 (A1-4), 404 to 424 (A1-5), 601 to 621 (A1-6) and 757 to 777 (A1-7). Five siRNAs were directed at the hnRNP A2 mRNA (GenBank accession number NM_{—}002137) and were from nucleotides 48 to 68 (A2-1), 57 to 77 (A2-2), 298 to 318 (A2-3), 615 to 635 (A2-4) and 922 to 942 (A2-5). Prior to transfection, siRNA duplexes were prepared by annealing complementary pairs of oligonucleotides. Duplex formation was verified by fractionating a portion of the mixture on a 2% agarose gel. The final concentration of the siRNA duplex was 50 μM in 20 mM KCl, 6 mM HEPES-KOH pH 7.5 and 0.2 mM MgCl₂. This mixture was stored frozen in aliquots at −80° C.

The sequence of the siRNAs are

A1#1
5′-UGGGGAACGCUCACGGACUdTdT-3′ (sense),
3′-dTdTACCCCUUGCGAGUGCCUGA-5′ (antisense),
A1#1M:
5′-UGGGGAACCGUCACGGACUdTdT-3′ (sense),
3′-dTdTACCCCUUGGCAGUGCCUGA-5′ (antisense),
A1#2:
5′-UGAGAGAUCCAAACACCAAdTdT-3′ (sense),
3′-dTdTACUCUCUAGGUUUGUGGUU-5′ (antisense),
A1#3:
5′-GCGCUCCAGGGGCUUUGGGdTdT-3′ (sense),
3′-dTdTCGCGAGGUCCCCGAAACCC-5′ (antisense),
A1#4:
5′-UCGAAGGCCACACAAGGUGdTdT-3′ (sense)
3′-dTdTAGCUUCCGGUGUGUUCCAC-5′ (antisense),
A1#5:
5′-AUCAUGACUGACCGAGGCAdTdT-3′ (sense),
3′-dTdTUAGUACUGACUGGCUCCGU-5′ (antisense),
A1#6:
5′-CUUUGGUGGUGGUCGUGGAdTdT-3′ (sense),
3′-dTdTGAAACCACCACCAGCACCU-5′ (antisense),
A1#7:
5′-UUUUGGAGGUGGUGGAAGCdTdT-3′ (sense),
3′-dTdTAAAACCUCCACCACCUUCG-5′ (antisense),
A2#1:
5′-GCUUUGAAACCACAGAAGAdTdT-3′ (sense),
3′-dTdTCGAAACUUUGGUGUCUUCU-5′ (antisense),
A2#2:
5′-CCACAGAAGAAAGUUUGAGdTdT-3′ (sense),
3′-dTdTGGUGUCUUCUUUCAAACUC-5′ (antisense),
A2#3:
5′-GAAGCUGUUUGUUGGCGGAdTdT-3′ (sense),
3′-dTdTCUUCGACAAACAACCGCCU-5′ (antisense),
A2#4:
5′-AUUUCGGACCAGGACCAGGdTdT-3′ (sense),
3′-dTdTUAAAGCCUGGUCCUGGUCC-5′ (antisense),
A2#5:
5′-CUUUGGUGGUAGCAGGAACdTdT-3′ (sense),
3′-dTdTGAAACCACCAUCGUCCUUG-5′ (antisense).

Transfection

The day before transfection, exponentially growing cells were trypsinized and seeded into 6-well plates. Transfection was performed on 30 to 50% confluent cells using Oligofectamine™ according to the manufacturer's instructions and at the indicated siRNA concentrations: HeLaS3 (80 nM), HCT 116 (20 or 40 nM), HCT 116 p53-(40 nM), HT-1080 (20 nM), PA-1 (10 nM), U-373 MG (10 nM), SK-OV-3 (20 nM) HIEC (80 nM), BJ (80 nM), BJ-TIELF (80 nM), and CCD-18Co (80 nM). Briefly, the siRNAs (in 10 μl) were mixed with 175 μl of OPTI-MEM-I (Invitrogen) while Oligofectamine™ was mixed with OPTI-MEM-I (4 μl and 11 μl, respectively). The transfection reagent and the siRNAs were then mixed and incubated at room temperature for 20 minutes before being applied to cells. A second transfection at the same concentration of siRNAs was always conducted 24 hours later.

TUNEL Assay and DNA Content Analysis

At the indicated time following the first transfection, both adherent and floating cells were harvested and counted. Cell viability was evaluated by trypan blue dye exclusion. The number of population doublings post-transfection was calculated for each sample using the equation: PD=log(Nf/N0)/log2.

TUNEL labeling was performed using the ApopTag kit™ (Intergen, S7110), according to the manufacturer's instructions. Briefly, adherent cells were fixed with 2% formaldehyde in PBSA for 1 hour at 4° C. and permeabilized in pre-cooled ethanol:acetic acid (2:1) for 5 minutes at −20° C. The reaction buffer containing the TdT enzyme was incubated on cells for 90 minutes at 37° C. in a wet chamber to create tails with digoxigenin-dNTP. The TdT products were detected using anti-digoxigenin conjugated with fluorescein for 30 minutes in a wet chamber at room temperature. Propidium iodide (0.5 μg/ml) was used as a nuclear couterstain to visualize the whole cell population. The cells were visualized by fluorescence microscopy.

For DNA content analysis, both floating and adherent cells were recovered, fixed in 80% cold ethanol, stand at room temperature for 5 minutes and stored at −20° C. (could be stored up to two weeks). The cells were washed with PBSA and treated with RNAse A for 30 minutes at 37° C. (20 μg RNAse A, 5 mM EDTA, 0.5% BSA in PBSA). The cells were stained with propidium iodide (50 μg) for 5 minutes at room temperature and read on a Becton Deckinson FACScan™ using the CellQuest™ software. For each sample, at least 10,000 cells were analyzed for DNA content.

Western Blotting

Whole cell extracts were prepared by lysing cells in Laemmli sample buffer. Equal amounts of each sample (15 to 25 μg) were loaded onto a polyacrylamide gel. A polyclonal rabbit anti-hnRNP A1/A2 antibody was used which preferentially recognizes the hnRNP A2 protein even when the hnRNP A1 and A2 proteins are present in equimolar amounts. Western blotting was performed according to standard protocols using the following dilutions for primary antibodies: 1:5000 for the anti-A1/A2 antibodies; 1:500 for the anti-PARP antibodies (Biosource, AHF0262); 1:100 for the active caspase-3 antibodies (Chemicon, AB3623); and 1:500 for the anti-pro-caspase-3 antibody (Biosource, AHZ52). Ponceau S-staining of the nitrocellulose membrane was used to confirm equal protein loading.

Telomere G-Tail Extension Analysis

The T-OLA assay was carried out as described in Cimino-Reale et al., Nucl. Acids Res. 29:e35, 2001. Briefly, genomic DNA was prepared from by standard cell lysis protocols. Oligonucleotide (CCCTAA)₃ was end-labeled and phosphorylated by T4 polynucleotide kinase in the following reaction mixture: 0.16 μM of oligonucleotide, 1.6 μM of [γ-32P]ATP (3000 Ci/mmole, 10 mCi/ml), 70 mM Tris pH 7.6, 10 mM MgCl2, 5 mM DTT and 20U of T4 polynucleotide kinase in a final volume of 50 μl. The reaction was allowed to proceed for 40 minutes at 37° C., then 1 μl of 0.1 M ATP and a further 10U of kinase were added before another 20 minutes incubation period. The enzyme was then heat-inactivated at 65° C. for 20 minutes. The oligonucleotide was precipitated with ethanol and dissolved in water. Hybridization was conducted in a 20 μl volume containing 10 μg of undenatured DNA, 0.5 pmole of oligonucleotide, 20 mM Tris pH 7.6, 25 mM potassium acetate, 10 mM magnesium acetate, 10 mM DTT, 1 mM nicotinamide adenine dinucleotide (NAD) and 0.1% Triton X-100 in a 0.5 ml PCR tube at 50° C. for 12 to 14 hours. Forty units of thermostable Taq ligase (New England Biolabs) and 2 μl of fresh 10 mM NAD stock were added and the ligation reaction was allowed to proceed for 5 hours at the same temperature. Reactions were ended by adding 30 μl of water and by phenol-chloroform extraction. Samples were ethanol-precipitated and dissolved in 6 μl of TE buffer. Three μl of each reaction was mixed with 4 μl of formamide dye, denatured by heating at 90° C. and quenched on ice before loading onto a 8% acrylamide-urea gel. Gel were exposed to an autoradiography film before the ligation products were scanned and quantified.

Selection of a Human Cell Line that Expresses the Mouse A1 Protein

HeLa S3 cells were transfected with a plasmid encoding a myc-tagged mouse A1 cDNA using DOSPER Liposomal Transfection Reagent (Roche). Two days after transfection, cells were reseeded at a lower density and 400 μg/ml of Geneticin (Invitrogen) was added for positive selection of transfected cells. Clonal zones were individually reseeded in 24-wells plate and screened for myc-A1 protein expression using a anti-myc antibody (FIG. 24B). In addition to the human A2 protein, the resultant positive clones therefore expressed the human and mouse A1 proteins.

Nucleic Acid Molecules of the Invention

A nucleoside is a nucleobase-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; open linear structures are generally preferred. Within the oligonucleotide structure, the phosphate groups are commonly referred to as forming the backbone of the oligonucleotide. The normal linkage or backbone of RNA and DNA is a 3′ to 5′ phosphodiester linkage. As used herein, “nucleic acid molecule” includes any natural or non-natural nucleoside, nucleotide, or nucleobase oligomer.

Specific examples of preferred nucleobase oligomers useful in this invention include oligonucleotides containing modified backbones or non-natural internucleoside linkages. As defined in this specification, nucleobase oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers.

Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity, wherein the adjacent pairs of nucleoside units are linked 3′-5′ to 5′-3′ or 2′-5′ to 5′-2′. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other nucleobase oligomers, both the sugar and the internucleoside linkage, i.e., the backbone, are replaced with novel groups. One such nucleobase oligomer, is referred to as a Peptide Nucleic Acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Methods for making and using these nucleobase oligomers are described, for example, in “Peptide Nucleic Acids: Protocols and Applications” Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

In particular embodiments of the invention, the nucleobase oligomers have phosphorothioate backbones and nucleosides with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂—, and —O—N(CH₃)—CH₂—CH₂—. In other embodiments, the oligonucleotides have morpholino backbone structures described in U.S. Pat. No. 5,034,506.

Nucleobase oligomers may also contain one or more substituted sugar moieties. Nucleobase oligomers comprise one of the following at the 2′ position: OH; F; O-, S-, or N-alkyl; O-, S-, or N-alkenyl; O-, S- or N--alkynyl; or O-alkyl-O-alkyl, wherein the alkyl, alkenyl, and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred nucleobase oligomers include one of the following at the 2′ position: C₁ to C₁₀ lower alkyl, substituted lower alkyl, alkaryl, aralkyl, O-alkaryl, or O-aralkyl, SH, SCH₃, OCN, Cl, Br, CN, CF₃, OCF₃, SOCH₃, SO₂CH₃, ONO₂, NO₂, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleobase oligomer, or a group for improving the pharmacodynamic properties of an nucleobase oligomer, and other substituents having similar properties. Preferred modifications are 2′-O-methyl and 2′-methoxyethoxy (2′-O—CH₂CH₂OCH₃, also known as 2′-O-(2-methoxyethyl) or 2′-MOE). Another desirable modification is 2′-dimethylaminooxyethoxy (i.e., O(CH₂)₂ON(CH₃)₂), also known as 2′-DMAOE. Other modifications include, 2′-aminopropoxy (2′-OCH₂CH₂CH₂NH₂) and 2′-fluoro (2′-F). Similar modifications may also be made at other positions on an oligonucleotide or other nucleobase oligomer, particularly the 3′ position of the sugar on the 3′ terminal nucleotide or in 2′-5′ linked oligonucleotides and the 5′ position of 5′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleobase oligomers may also include nucleobase modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo (e.g., 5-bromo), 5-trifluoromethyl and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of an antisense oligonucleotide of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications, CRC Press, Boca Raton, 1993, pp. 276-278) and are desirable base substitutions, even more particularly when combined with 2′-O-methoxyethyl or 2′-O-methyl sugar modifications. Representative United States patents that teach the preparation of certain of the above noted modified nucleobases as well as other modified nucleobases include U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692, each of which is herein incorporated by reference.

Another modification of a nucleobase oligomer of the invention involves chemically linking to the nucleobase oligomer one or more moieties or conjugates that enhance the activity, cellular distribution, or cellular uptake of the oligonucleotide. Such moieties include but are not limited to lipid moieties such as a cholesterol moiety (Letsinger et al., Proc. Natl. Acad. Sci. USA, 86: 6553-6556, 1989), cholic acid (Manoharan et al., Bioorg. Med. Chem. Let, 4: 1053-1060, 1994), a thioether, e.g., hexyl-S-tritylthiol (Manoharan et al., Ann. N.Y. Acad. Sci., 660: 306-309, 1992; Manoharan et al., Bioorg. Med. Chem. Let., 3: 2765-2770, 1993), a thiocholesterol (Oberhauser et al., Nucl. Acids Res., 20: 533-538: 1992), an aliphatic chain, e.g., dodecandiol or undecyl residues (Saison-Behmoaras et al., EMBO J., 10: 1111-1118, 1991; Kabanov et al., FEBS Lett., 259: 327-330, 1990; Svinarchuk et al., Biochimie, 75: 49-54, 1993), a phospholipid, e.g., di-hexadecyl-rac-glycerol or triethylammonium 1,2-di-O-hexadecyl-rac-glycero-3-H-phosphonate (Manoharan et al., Tetrahedron Lett., 36: 3651-3654, 1995; Shea et al., Nucl. Acids Res., 18: 3777-3783, 1990), a polyamine or a polyethylene glycol chain (Manoharan et al., Nucleosides & Nucleotides, 14: 969-973, 1995), or adamantane acetic acid (Manoharan et al., Tetrahedron Lett., 36: 3651-3654, 1995), a palmityl moiety (Mishra et al., Biochim. Biophys. Acta, 1264: 229-237, 1995), or an octadecylamine or hexylamino-carbonyl-oxycholesterol moiety (Crooke et al., J. Pharmacol. Exp. Ther., 277: 923-937, 1996. Representative United States patents that teach the preparation of such nucleobase oligomer conjugates include U.S. Pat. Nos. 4,587,044; 4,605,735; 4,667,025; 4,762,779; 4,789,737; 4,824,941; 4,828,979; 4,835,263; 4,876,335; 4,904,582; 4,948,882; 4,958,013; 5,082,830; 5,109,124; 5,112,963; 5,118,802; 5,138,045; 5,214,136; 5,218,105; 5,245,022; 5,254,469; 5,258,506; 5,262,536; 5,272,250; 5,292,873; 5,317,098; 5,371,241, 5,391,723; 5,414,077; 5,416,203, 5,451,463; 5,486,603; 5,510,475; 5,512,439; 5,512,667; 5,514,785; 5,525,465; 5,541,313; 5,545,730; 5,552,538; 5,565,552; 5,567,810; 5,574,142; 5,578,717; 5,578,718; 5,580,731; 5,585,481; 5,587,371; 5,591,584; 5,595,726; 5,597,696; 5,599,923; 5,599,928; 5,608,046; and 5,688,941, each of which is herein incorporated by reference.

The present invention also includes nucleobase oligomers that are chimeric compounds. “Chimeric” nucleobase oligomers are nucleobase oligomers, particularly oligonucleotides, that contain two or more chemically distinct regions, each made up of at least one monomer unit, i.e., a nucleotide in the case of an oligonucleotide. These nucleobase oligomers typically contain at least one region where the nucleobase oligomer is modified to confer, upon the nucleobase oligomer, increased resistance to nuclease degradation, increased cellular uptake, and/or increased binding affinity for the target nucleic acid. An additional region of the nucleobase oligomer may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of nucleobase oligomer inhibition of gene expression. Consequently, comparable results can often be obtained with shorter nucleobase oligomers when chimeric nucleobase oligomers are used, compared to phosphorothioate deoxyoligonucleotides hybridizing to the same target region.

Chimeric nucleobase oligomers of the invention may be formed as composite structures of two or more nucleobase oligomers as described above. Such nucleobase oligomers, when oligonucleotides, have also been referred to in the art as hybrids or gapmers. Representative United States patents that teach the preparation of such hybrid structures include U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The nucleobase oligomers used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The nucleobase oligomers of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The nucleobase oligomers of the invention encompass any pharmaceutically acceptable salts, esters, or salts of such esters, or any other compound that, upon administration to an animal, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention can be prepared as SATE [(S-acetyl-2-thioethyl)phosphate] derivatives according to the methods disclosed in PCT publication Nos. WO 93/24510 or WO 94/26764.

The present invention also includes pharmaceutical compositions and formulations that include the nucleobase oligomers of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral, or parenteral. Parenteral administration includes intravenous, intraarterial, subcutaneous, intraperitoneal, or intramuscular injection or infusion; or intracranial, e.g., intrathecal or intraventricular, administration.

Locked Nucleic Acids

Locked nucleic acids (LNAs) are nucleobase oligomers that can be employed in the present invention. LNAs contain a 2′O, 4′-C methylene bridge that restrict the flexibility of the ribofuranose ring of the nucleotide analog and locks it into the rigid bicyclic N-type conformation. LNAs show improved resistance to certain exo- and endonucleases and activate RNAse H, and can be incorporated into almost any nucleobase oligomer. Moreover, LNA-containing nucleobase oligomers can be prepared using standard phosphoramidite synthesis protocols. Additional details regarding LNAs can be found in PCT publication No. WO 99/14226 and U.S. Patent Application Publication No. U.S. 2002/0094555 A1, each of which is hereby incorporated by reference.

Arabinonucleic Acids

Arabinonucleic acids (ANAs) can also be employed in methods and reagents of the present invention. ANAs are nucleobase oligomers based on D-arabinose sugars instead of the natural D-2′-deoxyribose sugars. Underivatized ANA analogs have similar binding affinity for RNA as do phosphorothioates. When the arabinose sugar is derivatized with fluorine (2′ F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars. The use of ANAs in therapy is discussed, for example, in Damha et al., Nucleosides Nucleotides & Nucleic Acids 20: 429-440, 2001.

RNA Interference

RNAi is a form of post-transcriptional gene silencing initiated by the introduction of double-stranded RNA (dsRNA). Short 21 to 25 nucleotide double-stranded RNAs are effective at down-regulating gene expression in nematodes (Zamore et al., Cell 101: 25-33) and in mammalian tissue culture cell lines (Elbashir et al., Nature 411: 494-498, 2001, hereby incorporated by reference). The further therapeutic effectiveness of this approach in mammals was demonstrated in vivo by McCaffrey et al. (Nature 418: 38-39. 2002). The nucleic acid sequence of a mammalian gene, such as A1 or A2, or the alternatively spliced isoforms A1^{B} or B1, can be used to design small interfering RNAs (siRNAs) that will inactivate the A1 or A2, or the alternatively spliced isoforms A1^{B} or B1, to which the siRNAs were directed. In a preferred embodiment, the siRNA to A1 will target a region of the gene that is common to both A1 and the splice variant A1^{B} and therefore reduce expression of both gene products. Similarly, in a preferred embodiment, the siRNA to A2 will target a region of the gene that is common to both A2 and the splice variant B1, and will therefore reduce expression of both gene products.

Provided with the sequence of a mammalian gene, dsRNAs may be designed to inactivate target genes of interest and screened for effective gene silencing, as described herein. In addition to the dsRNAs disclosed herein, additional dsRNAs may be designed using standard methods.

The specific requirements and modifications of dsRNA are described in PCT application number WO 01/75164 (incorporated herein by reference). While dsRNA molecules can vary in length, it is most preferable to use siRNA molecules that are 21- to 23-nucleotide dsRNAs with characteristic 2- to 3-nucleotide 3′ overhanging ends, preferably these are (2′-deoxy)thymidine or uracil. The siRNAs typically comprise a 3′ hydroxyl group. Alternatively, single stranded siRNAs or blunt ended dsRNA are used. In order to further enhance the stability of the RNA, the 3′ overhangs are stabilized against degradation. In one embodiment, the RNA is stabilized by including purine nucleotides, such as adenosine or guanosine. Alternatively, substitution of pyrimidine nucleotides by modified analogs e.g. substitution of uridine 2-nucleotide overhangs by (2′-deoxy)thymide is tolerated and does not affect the efficiency of RNAi. The absence of a 2′ hydroxyl group significantly enhances the nuclease resistance of the overhang in tissue culture medium.

siRNA molecules can be obtained through a variety of protocols including chemical synthesis or recombinant production using a Drosophila in vitro system. They can be commercially obtained from companies such as Dharmacon Research Inc. or Xeragon Inc., or they can be synthesized using commercially available kits such as the Silencer™ siRNA Construction Kit from Ambion (catalog number 1620) or HiScribe™ RNAi Transcription Kit from New England BioLabs (catalog number E2000S).

Alternatively siRNA can be prepared using any of the methods set forth in PCT number WO01/75164 (incorporated herein by reference) or using standard procedures for in vitro transcription of RNA and dsRNA annealing procedures as described in Elbashir S. M. et al. (Genes & Dev., 15: 188-200, 2001). siRNAs are also obtained as described in Elbashir S. M. et al. by incubation of dsRNA that corresponds to a sequence of the target gene in a cell-free Drosophila lysate from syncytial blastoderm Drosophila embryos under conditions in which the dsRNA is processed to generate siRNAs of about 21 to about 23 nucleotides, which are then isolated using techniques known to those of skill in the art. For example, gel electrophoresis can be used to separate the 21-23 nt RNAs and the RNAs can then be eluted from the gel slices. In addition, chromatography (e.g. size exclusion chromatography), glycerol gradient centrifugation, and affinity purification with antibody can be used to isolate the 21 to 23 nucleotide RNAs.

Short hairpin RNAs (shRNAs) can also be used for RNAi as described in Yu et al. or Paddison et al. (Proc. Natl. Acad. Sci USA, 99: 6047-6052, 2002; Genes & Dev, 16: 948-958, 2002; incorporated herein by reference). shRNAs are designed such that both the sense and antisense strands are included within a single RNA molecule and connected by a loop of nucleotides (3 or more). shRNAs can be synthesized and purified using standard in vitro T7 transcription synthesis as described above and in Yu et al. (supra). shRNAs can also be subcloned into an expression vector that has the mouse U6 promoter sequences which can then be transfected into cells and used for in vivo expression of the shRNA.

Introduction of dsRNA into Cells

The success of RNAi depends on a number of factors including dsRNA sequence selection and design, the cells being used, transfection reagents and transfection conditions. A variety of methods are available for transfection, or introduction, of dsRNA into mammalian cells. For example, there are several commercially available transfection reagents including but not limited to: TransIT-TKO™ (Mirus, Cat. # MIR 2150), Transmessenger™ (Qiagen, Cat. # 301525), and Oligofectamine™ (Invitrogen, Cat. # MIR 12252-011). Protocols for each transfection reagent are available from the manufacturer.

The concentration of dsRNA used for each target and each cell line varies but in general ranges from 0.05 nM to 500 nM, more preferably 0.1 nM to 100 nM, and most preferably 1 nM to 50 nM. If desired, cells can be transfected multiple times, using multiple dsRNAs to optimize the gene-silencing effect.

Stable Expression of siRNA

DNA template methods are used to create and deliver siRNA molecules (reviewed in T. Tuschl, Nature Biotechnology, 20: 446-448, 2002). The siRNA template is cloned into RNA polymerase III transcription units, which normally encode the small nuclear RNA U6 or the human RNAse P RNA H1. These expression cassettes allow for the expression of both sense and anti-sense RNA. Expression cassettes are also available for the stable expression of small hairpin RNAs (see Brummelkamp et al., Science 296: 550-553, 2002; Paddison et al., Genes & Dev. 16: 948-958, 2002; Paul et al., Nature Biotechnol. 20: 505-508, 2002; and Yu et al., Proc. Natl. Acad. Sci. USA 99(9): 6047-6052.

The endogenous expression of siRNA or shRNAs from introduced DNA templates is thought to overcome some limitations of exogenous delivery, in particular the transient loss of phenotype. In fact, stable cell lines have been obtained using these expression cassettes allowing for a stable loss of function phenotype (Miyagishi M. and Taira K., Nature Biotech., 20: 497-500, 2002; Brummelkamp T. R. et al., Science, 296: 550-553, 2002). shRNAs can also be expressed stably using a mouse U6 promoter based expression vector. If desired, stable cell lines for RNAi of A1 and/or A2 can be generated using the above techniques.

Assays for Evaluating Gene Silencing Effect

In general, cells are incubated for 5 hours to 7 days after transfection of siRNA and then harvested for analysis. mRNA and protein expression can be analyzed using any of a variety of art known methods including but not limited to northern blot analysis, RNAse protection assays, luciferase or 13-gal reporter assays, and western blots.

Cell Types

RNAi is used to downregulate gene or protein expression of A1, A1^{B}, A2, or B1, or any combination thereof, in virtually any mammalian cell expressing A1, A1^{B}, A2, or B1. In preferred embodiments, one siRNA will downregulate the expression of both A1 and A1^{B}, and another siRNA will downregulate the expression of both A2 and B1. These cells include, but are not limited to, HeLaS3, HCT116, CCD18Co, BJ, BJ-TIELF, HIEC, NIH3T3, BHK-21, 4T1, F9, P19, J774A.1, CHO-K1, primary human mammary epithelial cells, and neoplastic cells, which express higher levels of A1 than differentiating tissues (Biamonti et al. J. Mol. Biol. 230: 77-89, 1993).

Assays for Evaluating Promotion of Cell Death

The effectiveness of A1/A1^{B} and/or A2/B1 targeted RNAi in promoting cell death is assayed using any assay systems known in the art, including but not limited to, standard cell growth assays, trypan blue staining for cell survival, TUNEL assays, flow cytometry analysis, detection of apoptotis markers by western blot, or any other assay for apoptosis.

Assays for Evaluating Telomere Length

The effectiveness of A1/A1^{B} and/or A2/B1 targeted RNAi in modulating telomere length can be assayed using virtually any assay for telomere length known in the art, including, but not limited to, Southern blotting with oligonucleotides that are homologous to telomeric sequences in order to measure telomere restriction fragment (TRF) length or Oligonucleotide Ligation Assays (OLA) to measure the telomeric G-rich strand 3′single-stranded overhang.

Diagnostics

Expression levels of particular nucleic acids or polypeptides may be correlated with a particular disease state, and thus are useful in diagnosis. Oligonucleotides or longer fragments derived from hnRNP A1, A1^{B}, A2, or B1 may be used as probes to assay the expression levels of an endogenous hnRNP A1, A1^{B}, A2, or B1 in a biological sample (e.g., isolated cell, isolated tissue, biopsy specimen, or biological fluid) from a subject (e.g., patient). Biological samples showing increased levels of hnRNP A1, A1^{B}, A2, or B1, or any combination thereof, relative to a corresponding control sample diagnose the patient as having or having a propensity to develop a neoplasia (e.g., lung cancer, colon cancer, kidney cancer, bone cancer, breast cancer, prostate cancer, uterine cancer, ovarian cancer, liver cancer, pancreatic cancer, brain cancer, lymphoma, melanoma, myeloma, adenocarcinoma, thymoma, plasmacytoma, or any other neoplasm). Preferably, a subject having a neoplasia or having a propensity to develop a neoplasia will show an increase in the expression of at least one of hnRNP A1, hnRNP A1^{B}, hnRNP A2, or hnRNP B1.

In another embodiment, an antibody that specifically binds at least one of hnRNP A1, A1^{B}, A2, or B1 polypeptides, may be used for the diagnosis of a neoplasia. A variety of protocols for measuring an alteration in the expression of such polypeptides are known, including immunological methods (such as ELISAs and RIAs), and provide a basis for diagnosing a neoplasia. An increase in the level of at least one of hnRNP A1, A1B, A2, or B1 polypeptide is diagnostic of a patient having a neoplasia.

In yet another embodiment, hybridization with PCR probes that are capable of detecting an hnRNP A1/A1^{B} or hnRNP A2/B1, or both, polynucleotide sequences, including genomic sequences, or closely related molecules, may be used to hybridize to a nucleic acid sequence derived from a patient having a neoplasia. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations indicative of a neoplasia in hnRNP A1/A1^{B} or hnRNP A2/B1 gene or may be used to monitor expression levels of these genes (for example, by Northern analysis (Ausubel et al., Current Protocols in Molecular Biology, Wiley Interscience, New York, 2001).

In yet another approach, a subject may be diagnosed for a propensity to develop a neoplasia by direct analysis of the sequence of an hnRNP A1/A1^{B} or hnRNP A2/B1 nucleic acid molecules.

Screening Assays

As discussed above, the expression of an hnRNP A1/A1^{B} or hnRNP A2/B1 gene is increased in neoplasia. Based on this discovery, compositions of the invention are useful for the high-throughput low-cost screening of candidate compounds to identify those that decrease the expression or biological activity of an hnRNP A1/A1^{B} and/or hnRNP A2/B1 polypeptide whose expression is increased in a patient having a neoplasia.

Any number of methods are available for carrying out screening assays to identify new candidate compounds that inhibit the expression of an hnRNP A1/A1^{B} and/or hnRNP A2/B1 polypeptide. In one working example, candidate compounds are added at varying concentrations to the culture medium of cultured cells expressing an an hnRNP A1/A1^{B} and/or hnRNP A2/B1 nucleic acid sequence. Gene expression is then measured, for example, by microarray analysis, Northern blot analysis (Ausubel et al., supra), or RT-PCR, using any appropriate fragment prepared from the nucleic acid molecule as a hybridization probe. The level of gene expression in the presence of the candidate compound is compared to the level measured in a control culture medium lacking the candidate molecule. A compound which promotes a decrease in the expression of an an hnRNP A1/A1^{B} and/or hnRNP A2/B1 nucleic acid molecule, or a functional equivalent thereof, is considered useful in the invention; such a candidate compound may be used, for example, as a therapeutic to treat a neoplasia in a human patient.

In another working example, the effect of candidate compounds may be measured at the level of polypeptide production using the same general approach and standard immunological techniques, such as Western blotting or immunoprecipitation with an antibody specific for a polypeptide encoded by an hnRNP A1/A1^{B} and/or hnRNP A2/B1 gene. For example, immunoassays may be used to detect or monitor the expression of an hnRNP A1/A1^{B} and/or hnRNP A2/B1 polypeptide in an organism. Polyclonal or monoclonal antibodies that are capable of binding to such a polypeptide may be used in any standard immunoassay format (e.g., ELISA, Western blot, or RIA assay) to measure the level of the polypeptide. Preferably, a candidate compound promotes a decrease in the expression or biological activity of the polypeptide. Again, such a molecule may be used, for example, as a therapeutic to prevent, delay, ameliorate, or treat a neoplasia, or the symptoms of a neoplasia, in a human patient.

In yet another working example, candidate compounds may be screened for those that specifically bind to an hnRNP A1/A1^{B} and/or hnRNP A2/B1 polypeptide. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind an hnRNP A1/A1^{B} and/or hnRNP A2/B1 polypeptide. In another embodiment, a candidate compound is tested for its ability to enhance the biological activity of an hnRNP A1/A1^{B} and/or hnRNP A2/B1 polypeptide. The biological activity of an hnRNP A1/A1^{B} and/or hnRNP A2/B1 is assayed using standard methods as described herein.

In another working example, an hnRNP A1/A1^{B} and/or hnRNP A2/B1 nucleic acid molecule is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that decreases the expression of the detectable reporter is a compound that is useful for the treatment of a neoplasia. In preferred embodiments, the candidate compound decreases the expression of a reporter gene fused to an an hnRNP A1/A1^{B} and/or hnRNP A2/B1 nucleic acid molecule.

In one particular working example, a candidate compound that binds to an an hnRNP A1/A1^{B} and/or hnRNP A2/B1 polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the an hnRNP A1/A1^{B} and/or hnRNP A2/B1 polypeptide is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

Potential antagonists include organic molecules, peptides, peptide mimetics, polypeptides, nucleic acids, and antibodies that bind to an hnRNP A1/A1^{B} and/or hnRNP A2/B1 nucleic acid sequence or polypeptide.

Each of the DNA sequences listed herein may also be used in the discovery and development of a therapeutic compound for the treatment of neoplasia. The encoded protein, upon expression, can be used as a target for the screening of drugs. Additionally, the DNA sequences encoding the amino terminal regions of the encoded protein or Shine-Delgamo or other translation facilitating sequences of the respective mRNA can be used to construct sequences that promote the expression of the coding sequence of interest. Such sequences may be isolated by standard techniques (Ausubel et al., supra).

Small molecules of the invention preferably have a molecular weight below 2,000 daltons, more preferably between 300 and 1,000 daltons, and most preferably between 400 and 700 daltons. It is preferred that these small molecules are organic molecules.

Since we have discovered that siRNA targeted against hnRNP A1/A1^{B} and hnRNP A2/B1 has a similar effect in mouse and human neoplastic cell lines, mouse cells can be used for any of the screening assays described herein or to test any of the therapeutic compounds identified. In addition, a mouse tumor model can be used as a model to test the safety and efficacy of potential therapeutic compounds.

Test Extracts and Compounds

In general, compounds that decrease hnRNP A1/A1^{B} and/or hnRNP A2/B1 expression or biological activity are identified from large libraries of both natural products, synthetic (or semi-synthetic) extracts or chemical libraries, according to methods known in the art. Those skilled in the art will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modifications of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from, for example, Brandon Associates (Merrimack, N.H.), Aldrich Chemical (Milwaukee, Wis.), and Talon Cheminformatics (Acton, Ont.)

Alternatively, libraries of natural compounds in the form of bacterial, fungal, plant, and animal extracts are commercially available from a number of sources, including, but not limited to, Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art (e.g., by combinatorial chemistry methods or standard extraction and fractionation methods). Furthermore, if desired, any library or compound may be readily modified using standard chemical, physical, or biochemical methods.

hnRNP A1/A1B or hnRNP A2/B1 Production

hnRNP A1/A1^{B} or hnRNP A2/B1 polypeptides are useful in screening for candidate compounds that bind to such polypeptides and inhibit their biological activity. In general, polypeptides, such as an hnRNP A1/A1^{B} or hnRNP A2/B1, may be produced by transformation of a suitable host cell, for example, a eukaryotic cell, with all or part of a polypeptide-encoding nucleic acid molecule, or a fragment thereof in a suitable expression vehicle.

Those skilled in the field of molecular biology will understand that any of a wide variety of expression systems may be used to provide the recombinant protein. Eukaryotic hnRNP A1/A1^{B} or hnRNP A2/B1 peptide expression systems may be generated in which an hnRNP A1/A1^{B} or hnRNP A2/B1 gene sequence is introduced into a plasmid or other vector, which is then used to transform living cells. Constructs in which the hnRNP A1/A1^{B} or hnRNP A2/B1 cDNA containing the entire open reading frame inserted in the correct orientation into an expression plasmid may be used for protein expression. Eukaryotic expression systems allow for the expression and recovery of hnRNP A1/A1^{B} or hnRNP A2/B1 peptide fusion proteins in which the hnRNP A1/A1^{B} or hnRNP A2/B1 peptide is covalently linked to a tag molecule that facilitates identification and/or purification. An enzymatic or chemical cleavage site can be engineered between the hnRNP A1/A1^{B} or hnRNP A2/B1 peptide and the tag molecule so that the tag can be removed following purification.

Typical expression vectors contain promoters that direct the synthesis of large amounts of mRNA corresponding to the inserted hnRNP A1/A1^{B} or hnRNP A2/B1 nucleic acid in the plasmid-bearing cells. They may also include an origin of replication sequence allowing for their autonomous replication within the host organism, sequences that encode genetic traits that allow vector-containing cells to be selected for in the presence of otherwise sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

The precise host cell used is not critical to the invention. A hnRNP A1/A1^{B} or hnRNP A2/B1 polypeptide may be produced in any eukaryotic host (e.g., Saccharomyces cerevisiae, insect cells, such as Sf21 cells, or mammalian cells, such as NIH 3T3, HeLa, COS cells, or fibroblasts). Such cells are available from a wide range of sources (e.g., the American Type Culture Collection, Rockland, Md.; also, see, e.g., Ausubel et al., Current Protocols in Molecular Biology, Wiley Interscience, New York, 2001). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

Native hnRNP A1/A1^{B} or hnRNP A2/B1 can be isolated from human cells that produce it naturally, or from transgenic eukaryotic cells that have been engineered to express a recombinant hnRNP A1/A1^{B} or hnRNP A2/B1 gene.

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). The recombinant protein can be purified by any appropriate techniques, including, for example, high performance liquid chromatography chromatography or other chromatographies (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs.

Therapeutic hnRNP A1 or hnRNP A2 RNAi

Neoplasms from any warm-blooded mammal may be treated using the methods of the invention. Neoplasms subject to such therapies include, but are not limited to, lung cancer, colon cancer, kidney cancer, bone cancer, breast cancer, prostate cancer, uterine cancer, ovarian cancer, liver cancer, pancreatic cancer, brain cancer, lymphoma, melanoma, myeloma, adenocarcinoma, thymoma, plasmacytoma, or any other neoplasm, such neoplasms are, preferably, characterized by having increased hnRNP A1/A1^{B} and/or hnRNP A2/B1 expression. Of particular interest for using the dsRNA molecules of the invention are neoplasms associated with increased expression of the hnRNP gene product or expression of an altered gene product. Warm-blooded animals include, but are not limited to, humans, cows, horses, pigs, sheep, birds, mice, rats, dogs, cats, monkies, baboons, or other mammals.

Therapy may be provided wherever cancer therapy is performed: at home, the doctor's office, a clinic, a hospital's outpatient department, or a hospital. Treatment generally begins at a hospital so that the doctor can observe the therapy's effects closely and make any adjustments that are needed. The duration of the therapy depends on the kind of cancer being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods so that the patient's body has a chance to build healthy new cells and regain its strength.

Depending on the type of cancer and its stage of development, the therapy can be used to slow the spreading of the cancer, to slow the cancer's growth, to kill or arrest neoplastic cells that may have spread to other parts of the body from the original tumor, to relieve symptoms caused by the cancer, or to prevent cancer in the first place.

As used herein, the terms “cancer” or “neoplasm” or “neoplastic cells” is meant a collection of cells multiplying in an abnormal manner. Cancer growth is uncontrolled and progressive, and occurs under conditions that would not elicit, or would cause cessation of, multiplication of normal cells.

A nucleic acid molecule of the invention may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracistemal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a nucleic acid molecule of the invention is likely to depend on such variables as the type and extent of the disorder, the overall health status of the particular patient, the formulation of the compound excipients, and its route of administration.

RNAi Therapeutics Directed to hnRNP A1/A1B or hnRNP A2/B1

The administration of hnRNP A1/A1^{B} and/or hnRNP A2/B1 nucleic acid molecules for RNAi therapy (e.g., dsRNA, antisense RNA, or siRNA) may be provided to prevent or treat a neoplasm. Such nucleic acid molecules will downregulate the expression of either hnRNP A1 or A1^{B}, preferably both, and hnRNP A2 or B1, preferably both. Such nucleic acid molecules may be administered directly to a tissue or neoplasm or may be provided within an expression vector, such that the nucleic acid molecule mediating the RNAi is stably expressed.

For direct administration of hnRNP A1/A1^{B} and/or hnRNP A2/B1 nucleic acid molecules for RNAi (e.g., dsRNA, antisense RNA, or siRNA) or mixtures thereof, nucleic acid molecules are provided in a unit dose form, each dose containing a predetermined quantity of such molecules sufficient to silence a target gene in association with a pharmaceutically acceptable diluent or carrier, such as phosphate-buffered saline, to form a pharmaceutical composition. In addition, the hnRNP A1/A1^{B} and/or hnRNP A2/B1 nucleic acid molecules for RNAi may be formulated in a solid form and redissolved or suspended prior to use. The pharmaceutical composition may, optionally, contain other chemotherapeutic agents, antibodies, antivirals, and exogenous immunomodulators.

In providing a mammal with the nucleic acid molecules for RNAi, the dosage of administered nucleic acid molecules will vary depending upon such factors as the mammal's age, weight, height, sex, general medical condition, previous medical history, disease progression, tumor burden, and the like. The dose is administered as indicated. Other therapeutic drugs may be administered in conjunction with the nucleic acid molecules.

The efficacy of treatment using the nucleic acid molecules described herein may be assessed by determination of alterations in the concentration or activity of the DNA, RNA or gene product of A1 or A1^{B} band A2 or B1, tumor regression, or a reduction of the pathology or symptoms associated with the neoplasm.

Antisense Nucleobase Oligomers Directed to hnRNP A1/A1^{B} and hnRNP A2/B1

The present invention also features the use of antisense nucleobase oligomers to downregulate expression of hnRNP A1/A1^{B} and/or hnRNP A2/B1. By binding to the complementary nucleic acid sequence (the sense or coding strand), antisense nucleobase oligomers are able to inhibit protein expression presumably through the enzymatic cleavage of the RNA strand by RNAse H. Preferably a combination of antisense nucleobase oligomers is used to reduce expression of at least one of hnRNP A1 or A1^{B} and at least one of hnRNP A2 or B1 in a cell that expresses increased levels of those proteins. More preferably, a combination of antisense nucleobase oligomers is used to reduce expression of all of hnRNP A1, hnRNP A1^{B}, hnRNP A2, and hnRNP B1 in a cell. Preferably the decrease in protein expression is at least 10% relative to cells treated with a control oligonucleotide, more preferably 25%, and most preferably 50% or greater. Methods for selecting and preparing antisense nucleobase oligomers are well known in the art. Methods for assaying levels of protein expression are also well known in the art and include western blotting, immunoprecipitation, and ELISA.

Nucleic Acid Therapy

Nucleic acid therapy methods, such as those described above, are used to prevent or ameliorate a neoplasia having increased expression of an hnRNP A1/A1^{B} or hnRNP A2/B1 nucleic acid molecule. Expression vectors encoding anti-sense nucleic acid molecules, dsRNAs, siRNAs, or shRNAs can be delivered to cells that overexpress an endogenous an hnRNP A1/A1^{B} and hnRNP A2/B1 nucleic acid molecule. Such delivery results in the sustained expression of the molecules tageting hnRNP A1/A1^{B} and hnRNP A2/B1 nucleic acid molecules for RNAi. The nucleic acid molecules must be delivered to cells in need of RNAi (e.g., neoplastic cells) in a form in which they can be taken up by the cells and so that sufficient levels of RNAi nucleic acid molecules can be produced to decrease hnRNP A1/A1^{B} or hnRNP A2/B1 levels in a patient having a neoplasia.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8: 423-430, 1997; Kido et al., Current Eye Research 15: 833-844, 1996; Bloomer et al., Journal of Virology 71: 6641-6649, 1997; Naldini et al., Science 272: 263-267, 1996; and Miyoshi et al., Proc. Natl. Acad. Sci. U.S.A. 94: 10319, 1997). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244: 1275-1281, 1989; Eglitis et al., BioTechniques 6: 608-614, 1988; Tolstoshev et al., Current Opinion in Biotechnology 1: 55-61, 1990; Sharp, The Lancet 337: 1277-1278, 1991; Cornetta et al., Nucleic Acid Research and Molecular Biology 36: 311-322, 1987; Anderson, Science 226: 401-409, 1984; Moen, Blood Cells 17: 407-416, 1991; Miller et al., Biotechnology 7: 980-990, 1989; Le Gal La Salle et al., Science 259: 988-990, 1993; and Johnson, Chest 107: 77S-83S, 1995). Retroviral vectors are particularly well developed and have been used in clinical settings (Rosenberg et al., N. Engl. J. Med 323: 370, 1990; Anderson et al., U.S. Pat. No. 5,399,346). Most preferably, a viral vector is used to express an hnRNP A1 or hnRNP A2 nucleic acid molecule capable of mediating RNAi.

Non-viral approaches can also be employed for the introduction of an RNAi therapeutic to a cell of a patient having a neoplasia. For example, a nucleic acid molecule can be introduced into a cell by administering the nucleic acid in the presence of lipofection (Felgner et al., Proc. Natl. Acad. Sci. U.S.A. 84: 7413, 1987; Ono et al., Neuroscience Letters 17: 259, 1990; Brigham et al., Am. J. Med. Sci. 298: 278, 1989; Staubinger et al., Methods in Enzymology 101: 512, 1983), asialoorosomucoid-polylysine conjugation (Wu et al., Journal of Biological Chemistry 263: 14621, 1988; Wu et al., Journal of Biological Chemistry 264: 16985, 1989), or by micro-injection under surgical conditions (Wolff et al., Science 247: 1465, 1990). Preferably the nucleic acid molecules are contained within plasmid vectors and are administered in combination with a liposome and protamine.

Nucleic acid molecule expression for use in RNAi gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types, such as tumor cells, can be used to direct the expression of a nucleic acid. The enhancers used can include, without limitation, those that are characterized as tissue- or cell-specific enhancers.

Combination Therapies

hnRNP A1/A1^{B} or hnRNP A2/B1 nucleic acids or polypeptides may be administered in combination with any other standard neoplasia therapy; such methods are known to the skilled artisan (e.g., Wadler et al., Cancer Res. 50: 3473-86, 1990), and include, but are not limited to, chemotherapy, hormone therapy, immunotherapy, radiotherapy, and any other therapeutic method used for the treatment of neoplasia. Combinations of the nucleic acid therapeutics of the invention may also be used. For example, the invention provides for the use of siRNA and antisense nucleobase oligomers targeting hnRNP A1/A1^{B} or hnRNP A2/B1. In one working example, siRNA can be used to downregulate A1/A1^{B} and an antisense nuclebbase oligomer can be used to downregulate hnRNP A2/B1.

### Other Embodiments

From the foregoing description, it is apparent that variations and modifications may be made to the invention described herein to adopt it to various usages and conditions. Such embodiments are also within the scope of the following claims.

All publications, patents, and patent applications mentioned in this specification are herein incorporated by reference to the same extent as if each independent publication or patent application was specifically and individually indicated to be incorporated by reference.

1. A purified siRNA molecule having at least one strand that is at least 95% complementary to at least a portion of any one of the sequences set forth in SEQ ID NOs: 27 (hnRNP A1), 31 (hnRNP A1), and 32 (hnRNP A1^{B}), or a splice variant or isoform thereof, wherein said siRNA can reduce the level of a nucleic acid having at least one of said sequences in a cell in which said nucleic acid is expressed.

2. The purified siRNA molecule of claim 1, wherein the siRNA has at least one strand that is 100% complementary to at least 18 consecutive nucleotides of the sequences set forth in SEQ ID NOs: 27, 31, or 32.

3. The purified siRNA molecule of claim 2, wherein the siRNA has at least one strand that is 100% complementary to at least 19 consecutive nucleotides of the sequences set forth in SEQ ID NOs: 27, 31, or 32.

4. The purified siRNA molecule of claim 3, wherein the siRNA has at least one strand that is 100% complementary to at least 20 consecutive nucleotides of the sequences set forth in SEQ ID NOs: 27, 31, or 32.

5. The purified siRNA molecule of claim 4, wherein the siRNA has at least one strand that is 100% complementary to at least 21 consecutive nucleotides of the sequences set forth in SEQ ID NOs: 27, 31, or 32.

6. The purified siRNA molecule of claim 1, wherein the siRNA has at least one strand that is 100% complementary to at least 18 consecutive nucleotides of both SEQ ID NOs: 27 and 32, or both SEQ ID NOs: 31 and 32.

7. The purified siRNA molecule of claim 1, wherein the siRNA has at least one strand that is 100% complementary to at least a portion of one of the following sequences: nucleotides 1 to 865 of SEQ ID NO: 27 and nucleotides 857 to 1769 of SEQ ID NO: 27.

8. A purified nucleic acid molecule comprising at least one strand that is at least 95% complementary to at least a portion of the sequences set forth in SEQ ID NOs: 28 or 33, or any splice variant or isoform thereof, wherein said nucleic acid molecule can reduce the levels of a nucleic acid having at least one of said sequences in a cell in which said nucleic acid is expressed.

9. The purified nucleic acid molecule of claim 8, wherein said nucleic acid molecule is an siRNA.

10. The purified nucleic acid molecule of claim 9, wherein said siRNA has at least one strand that is 100% complementary to at least 18 consecutive nucleotides of the sequences set forth in SEQ ID NOs: 28 or 33.

11. The purified nucleic acid molecule of claim 10, wherein said siRNA has at least one strand that is 100% complementary to at least 19 consecutive nucleotides of the sequences set forth in SEQ ID NOs: 28 or 33.

12. The purified nucleic acid molecule of claim 11, wherein said siRNA has at least one strand that is at least 100% complementary to at least 20 consecutive nucleotides of the sequences set forth in SEQ ID NOs: 28 or 33.

13. The purified nucleic acid molecule of claim 12, wherein said siRNA has at least one strand that is 100% complementary to at least 21 consecutive nucleotides of the sequences set forth in SEQ ID NOs: 28 or 33.

14. The purified nucleic acid molecule of claim 8, wherein said nucleic acid molecule is an siRNA having at least one strand that is 100% complementary to at least 18 consecutive nucleotides of both SEQ ID NOs: 28 and 33.

15. The purified nucleic acid molecule of claim 14, wherein said siRNA has at least one strand that is 100% complementary to at least 18 consecutive nucleotides of the following sequences: nucleotides 1 to 176 of SEQ ID NO: 28 and nucleotides 177 to 1714 of SEQ ID NO: 28.

16. The purified nucleic acid molecule of claim 8, wherein said nucleic acid molecule is an antisense nucleobase oligomer.

17. The purified nucleic acid molecule of claim 16, wherein said antisense nucleobase oligomer is 100% complementary to at least 10 consecutive nucleotides of any one of the sequences set forth in SEQ ID NOs: 28 and 33, and wherein said antisense nucleobase oligomer can reduce the level of a nucleic acid having at least one of said sequences in a cell in which said nucleic acid is expressed.

18. The purified nucleic acid molecule of claim 17, wherein said antisense nucleobase oligomer is 100% complementary to at least 20 consecutive nucleotides of any one of the sequences set forth in SEQ ID NOs: 28 and 33.

19. The purified nucleic acid molecule of claim 18, wherein said antisense nucleobase oligomers is 100% complementary to at least 30 consecutive nucleotides of any one of the sequences set forth in SEQ ID NOs: 28 and 33.

20. The purified nucleic acid molecule of claim 16, wherein said antisense nucleobase oligomer is 100% complementary to at least 30 consecutive nucleotides of both SEQ ID NOs: 28 and 33.

21. The purified nucleic acid molecule of claim 16, wherein said antisense nucleobase oligomer has at least one strand that is 100% complementary to at least 10 consecutive nucleotides of the following sequences: nucleotides 1 to 176 of SEQ ID NO: 28 and nucleotides 177 to 1714 of SEQ ID NO: 28.

22. A composition comprising (i) a first nucleic acid molecule having at least one strand that is at least 95% complementary to at least a portion of any one of the sequences set forth in SEQ ID NOs: 27, 31, and 32, and (ii) a second nucleic acid molecule having at least one strand that is complementary at least 95% complementary to at least a portion of any one of the sequences set forth in SEQ ID NOs: 28 and 33, wherein said first nucleic acid molecule reduces the level of a nucleic acid having at least one of the sequences set forth in SEQ ID NOs: 27, 31, and 32 in a cell, and said second nucleic acid molecule reduces the level of a nucleic acid having at least one of the sequences set forth in SEQ ID NOs: 28 and 33 in a cell.

23. The composition of claim 22, wherein said first and second nucleic acid molecules are double stranded molecules.

24. The composition of claim 22, wherein said first or said second nucleic acid molecules are siRNA molecules.

25. The composition of claim 24, wherein said first and second nucleic acid molecules are siRNA nucleic acid molecules.

26. The composition of claim 21, wherein said first and second nucleic acid molecules are antisense nucleobase oligomers.

27. The composition of claim 25, wherein said first nucleic acid molecule is an siRNA molecule that is 100% complementary to 18 to 25 consecutive nucleotides of any one of SEQ ID NOs: 27, 31, and 32, and said second nucleic acid molecule is an siRNA molecule that is 100% complementary to 18 to 25 consecutive nucleotides of any one of SEQ ID NOs: 28 and 33.

28. The composition of claim 25, wherein said first nucleic acid molecules is selected from the group consisting of SEQ ID NOs: 1-16 and 29-30, and said second nucleic acid molecule is selected from the group consisting of SEQ ID NOs: 17-26.

29. The composition of claim 25, wherein said first nucleic acid molecule is an siRNA molecule that is 100% complementary to 18 to 25 consecutive nucleotides of both SEQ ID NOs: 27 and 32, or both SEQ ID NOs: 31 and 32, and said second nucleic acid molecule is an siRNA molecule that is 100% complementary to 18 to 25 consecutive nucleotides of both SEQ ID NOs: 28 and 33.

30. The composition of claim 25 wherein said first nucleic acid molecule is an siRNA molecule that is 100% complementary to 18 to 25 consecutive nucleotides of nucleotides 1 to 865 of SEQ ID NO: 27 or nucleotides 857 to 1769 of SEQ ID NO: 27; and said second nucleic acid molecule is an siRNA molecule that is 100% complementary to 18 to 25 consecutive nucleotides of nucleotides 1 to 176 of SEQ ID NO: 28 or nucleotides 177 to 1714 of SEQ ID NO: 28.

31. The composition of claim 26, wherein said first nucleic acid molecule is an antisense nucleobase oligomer that is 100% complementary to at least 10 consecutive nucleotides of any one of SEQ ID NOs: 27, 31, and 32, and said second nucleic acid molecule is an antisense nucleobase oligomer that is complementary to at least 10 consecutive nucleotides of any one of SEQ ID NOs: 28 and 33.

32. The composition of claim 26, wherein said first nucleic acid molecule is an antisense nucleobase oligomer that is 100% complementary to at least 10 consecutive nucleotides of both SEQ ID NOs: 27 and 32 or both SEQ ID NOs: 31 and 32, and said second nucleic acid molecule is an antisense nucleobase oligomer that is complementary to at least 10 consecutive nucleotides of both SEQ ID NOs: 28 and 33.

33. The composition of claim 26, wherein said first nucleic acid molecule is an antisense nucleobase oligomer that is 100% complementary to at least 10 consecutive nucleotides of nucleotides 1 to 865 of SEQ ID NO: 27 or nucleotides 857 to 1769 of SEQ ID NO: 27, and said second nucleic acid molecule is an antisense nucleobase oligomer that is 100% complementary to at least 10 consecutive nucleotides of nucleotides 1 to 176 of SEQ ID NO: 28 or nucleotides 177 to 1714 of SEQ ID NO: 28

34. The composition of claim 22, wherein said composition further comprises a pharmaceutically acceptable carrier.

35. The composition of claim 22, wherein said first nucleic acid molecule induces apoptosis in a cell expressing one of the nucleic acid sequences set forth in SEQ ID NOs: 37, 21, and 32, and said second nucleic acid molecule induces apoptosis in a cell expressing one of the nucleic acid sequences set forth in SEQ ID NOs: 28 and 33.

36. A composition comprising at least one pair of double stranded nucleic acid molecules, wherein said at least one pair is selected from the following pairs of nucleic acid molecules: SEQ ID NOs: 1 and 2; 3 and 4; 5 and 6; 7 and 8; 9 and 10; 11 and 12; 13 and 14; 15 and 16; 17 and 18; 19 and 20; 21 and 22; 23 and 24; 25 and 26; and 29 and 30, and a pharmaceutically acceptable carrier.

37. The composition of claim 36, wherein said pair of nucleic acid molecules further comprises a base linker region.

38. The composition of claim 36, wherein said composition comprises at least two pairs of nucleic acid molecules, wherein the first of said at least two pairs of nucleic acid molecules are selected from the following pairs of nucleic acid molecules: SEQ ID NOs: 1 and 2; 3 and 4; 5 and 6; 7 and 8; 9 and 10; 11 and 12; 13 and 14; 15 and 16; and 29 and 30, and the second of said at least two pairs of nucleic acid molecules are selected from the following pairs of nucleic acid molecules: SEQ ID NOs: 17 and 18; 19 and 20; 21 and 22; 23 and 24; 25 and 26.

39. A pharmaceutical composition comprising at least one nucleic acid molecule selected from the group consisting of SEQ ID NOs: 18, 20, 22, 24, and 26.

40. A kit for the treatment of a neoplasia in a patient comprising a purified siRNA molecule of claim 1.

41. A kit for the treatment of a neoplasia in a patient comprising a purified nucleic acid molecule of claim 7.

42. A kit for the treatment of a neoplasia in a patient comprising (i) a first nucleic acid molecule having at one strand that is at least 95% complementary to at least a portion of any one of the sequences set forth in SEQ ID NOs: 27, 31, and 32, and (ii) a second nucleic acid molecule having at least one strand that is at least 95% complementary to at least a portion of any one of the sequences set forth in SEQ ID NOs: 28 and 33, wherein said first nucleic acid molecule reduces the level of at least one of the nucleic acid sequences set forth in SEQ ID NOs: 27, 31, and 32 in a cell, and said second nucleic acid molecule reduces the level of at least one of said nucleic acid sequences set forth in SEQ ID NOs: 28 and 33 in a cell.

43. The kit of claim 42, wherein said first nucleic acid molecule induces apoptosis in a cell expressing one of the nucleic acid sequences set forth in SEQ ID NOs: 37, 21, and 32, and said second nucleic acid molecule induces apoptosis in a cell expressing one of the nucleic acid sequences set forth in SEQ ID NOs: 28 and 33.

44. The kit of claim 42, wherein said first nucleic acid is selected from the following pairs of nucleic acid molecules: SEQ ID NOs: 1 and 2; 3 and 4; 5 and 6; 7 and 8; 9 and 10; 11 and 12; 13 and 14; 15 and 16; and 29 and 30, and said second nucleic acid molecules are selected from the following pairs of nucleic acid molecules: SEQ ID NOs: 17 and 18; 19 and 20; 21 and 22; 23 and 24; 25 and 26.

45. A kit for the treatment of a neoplasia in a patient comprising at least one nucleic acid molecule selected from the group consisting of SEQ ID NO: 18, 20, 22, 24, and 26.

46. A diagnostic kit for the diagnosis of a neoplasia in a patient comprising a nucleic acid sequence, or fragment thereof, said kit comprising (i) a first nucleic acid molecule having at one strand that is at least 95% complementary to at least a portion of any one of the sequences set forth in SEQ ID NOs: 27, 31, and 32, and (ii) a second nucleic acid molecule having at least one strand that is at least 95% complementary to at least a portion of any one of the sequences set forth in SEQ ID NOs: 28 and 33.

47. A vector comprising a purified siRNA molecule of claim 1.

48. The vector of claim 47, wherein the siRNA is 100% complementary to 18 to 25 consecutive nucleotides of the sequences set forth in SEQ ID NOs: 27, 31, or 32.

49. A vector comprising a purified nucleic acid molecule of claim 7.

50. The vector of claim 49, wherein said nucleic acid molecule is an siRNA molecule.

51. The vector of claim 49, wherein said nucleic acid molecule is an antisense nucleobase oligomer.

52. The vector of claim 50, wherein the siRNA molecule is 100% complementary to 18 to 25 consecutive nucleotides of any one of the sequences set forth in SEQ ID NOs: 28 and 33.

53. The vector of claim 51, wherein said antisense nucleobase oligomer is 100% complementary to at least 10 consecutive nucleotides of any one of the sequences set forth in SEQ ID NOs: 28 and 33.

54. A vector comprising (i) a first nucleic acid molecule positioned for expression and having at least one strand that is at least 95% complementary to at least a portion of any one of the sequences set forth in SEQ ID NOs: 27, 31, and 32, and (ii) a second nucleic acid molecule positioned for expression and having at least one strand that is at least 95% complementary to at least a portion of any one of the sequences set forth in SEQ ID NOs: 28 and 33, wherein said first nucleic acid molecule reduces the level of at least one of the nucleic acid sequences set forth in SEQ ID NOs: 27, 31, and 32 in a cell, and said second nucleic acid molecule reduces the level of at least one of said nucleic acid sequences set forth in SEQ ID NOs: 28 and 33 in a cell.

55. The vector of claim 54, wherein said first or second nucleic acid molecules are siRNA.

56. The vector of claim 55, wherein said first and said second nucleic acid molecules are siRNA.

57. The vector of claim 56, wherein the first siRNA molecule is 100% complementary to 18 to 25 consecutive nucleotides of any one of SEQ ID NOs: 27, 31, and 32, and the second siRNA molecule is 100% complementary to 18 to 25 consecutive nucleotides of any one of SEQ ID NOs: 28 and 33.

58. The vector of claim 56, wherein the first siRNA molecule is 100% complementary to 18 to 25 consecutive nucleotides of both SEQ ID NOs: 27 and 32 or both SEQ ID NOs: 31 and 32, and the second siRNA molecule is 100% complementary to 18 to 25 consecutive nucleotides of both SEQ ID NOs: 28 and 33.

59. The vector of claim 56, wherein said first or said second nucleic acid molecules are antisense nucleobase oligomers.

60. The vector of claim 59, wherein the first and second nucleic acid molecules are antisense nucleobase oligomers.

61. The vector of claim 59, wherein the first antisense nucleobase oligomer is 100% complementary to at least 10 consecutive nucleotides of any one of SEQ ID NOs: 27, 31, and 32, and the second antisense nucleobase oligomer is 100% complementary to at least 10 consecutive nucleotides of any one of SEQ ID NOs: 28 and 33.

62. The vector of claim 61, wherein the first antisense nucleobase oligomer is 100% complementary to at least 10 consecutive nucleotides of both SEQ ID NOs: 27 and 32 or both SEQ ID NOs: 31 and 32, and the second antisense nucleobase oligomer is 100% complementary to at least 10 consecutive nucleotides of both SEQ ID NOs: 28 and 33.


SEQUENCE LISTING (37 sequences)

SEQ ID NO: 1 - DNA, 21 bp, Homo sapiens
  modified_base 20,21: T at positions 20 and 21 are dT
uggggaacgcucacggacutt

SEQ ID NO: 2 - DNA, 21 bp, Homo sapiens
  modified_base 20, 21: T at positions 20 and 21 are dT
aguccgugagcguuccccatt

SEQ ID NO: 3 - DNA, 21 bp, Homo sapiens
  modified_base 20,21: T at positions 20 and 21 are dT
uggggaaccgucacggacutt

SEQ ID NO: 4 - DNA, 21 bp, Homo sapiens
  modified_base 20, 21: T at positions 20 and 21 are dT
aguccgugagcguuccccatt

SEQ ID NO: 5 - DNA, 21 bp, Homo sapiens
  modified_base 20, 21: T at positions 20 and 21 are dT
ugagagauccaaacaccaatt

SEQ ID NO: 6 - DNA, 21 bp, Homo sapiens
  modified_base 20, 21: T at positions 20 and 21 are dT
uugguguuuggaucucucatt

SEQ ID NO: 7 - DNA, 21 bp, Homo sapiens
  modified_base 20,21: T at positions 20 and 21 are dT
gcgcuccaggggcuuugggtt

SEQ ID NO: 8 - DNA, 21 bp, Homo sapiens
  modified_base 20, 21: T at positions 20 and 21 are dT
cccaaagccccuggagcgctt

SEQ ID NO: 9 - DNA, 21 bp, Homo sapiens
  modified_base 20,21: T at positions 20 and 21 are dT
ucgaaggccacacaaggugtt

SEQ ID NO: 10 - DNA, 21 bp, Homo sapiens
  modified_base 20, 21: T at positions 20 and 21 are dT
caccuuguguggccuucgatt

SEQ ID NO: 11 - DNA, 21 bp, Homo sapiens
  modified_base 20,21: T at positions 20 and 21 are dT
aucaugacugaccgaggcatt

SEQ ID NO: 12 - DNA, 21 bp, Homo sapiens
  modified_base 20, 21: T at positions 20 and 21 are dT
ugccucggucagucaugautt

SEQ ID NO: 13 - DNA, 21 bp, Homo sapiens
  modified_base 20,21: T at positions 20 and 21 are dT
cuuuggugguggucguggatt

SEQ ID NO: 14 - DNA, 21 bp, Homo sapiens
  modified_base 20, 21: T at positions 20 and 21 are dT
uccacgaccaccaccaaagtt

SEQ ID NO: 15 - DNA, 21 bp, Homo sapiens
  modified_base 20,21: T at positions 20 and 21 are dT
uuuuggaggugguggaagctt

SEQ ID NO: 16 - DNA, 21 bp, Homo sapiens
  modified_base 20, 21: T at positions 20 and 21 are dT
gcuuccaccaccuccaaaatt

SEQ ID NO: 17 - DNA, 21 bp, Homo sapiens
  modified_base 20,21: T at positions 20 and 21 are dT
gcuuugaaaccacagaagatt

SEQ ID NO: 18 - DNA, 21 bp, Homo sapiens
  modified_base 20, 21: T at positions 20 and 21 are dT
ucuucugugguuucaaagctt

SEQ ID NO: 19 - DNA, 21 bp, Homo sapiens
  modified_base 20,21: T at positions 20 and 21 are dT
ccacagaagaaaguuugagtt

SEQ ID NO: 20 - DNA, 21 bp, Homo sapiens
  modified_base 20, 21: T at positions 20 and 21 are dT
cucaaacuuucuucuguggtt

SEQ ID NO: 21 - DNA, 21 bp, Homo sapiens
  modified_base 20,21: T at positions 20 and 21 are dT
gaagcuguuuguuggcggatt

SEQ ID NO: 22 - DNA, 21 bp, Homo sapiens
  modified_base 20, 21: T at positions 20 and 21 are dT
uccgccaacaaacagcuuctt

SEQ ID NO: 23 - DNA, 21 bp, Homo sapiens
  modified_base 20,21: T at positions 20 and 21 are dT
auuucggaccaggaccaggtt

SEQ ID NO: 24 - DNA, 21 bp, Homo sapiens
  modified_base 20, 21: T at positions 20 and 21 are dT
ccugguccugguccgaaautt

SEQ ID NO: 25 - DNA, 21 bp, Homo sapiens
  modified_base 20, 21: t at positions 20 and 21 are dT
cuuuggugguagcaggaactt

SEQ ID NO: 26 - DNA, 21 bp, Homo sapiens
  modified_base 20, 21: T at positions 20 and 21 are dT
guuccugcuaccaccaaagtt

SEQ ID NO: 27 - DNA, 1769 bp, Homo sapiens
gagagggcgaaggtaggctggcagatacgttcgtcagcttgctcctttctgcccgtggacgccgccgaagaagcatcgttaaagtctctcttcaccctgccgtcatgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctctaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggcaatgatggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaa

SEQ ID NO: 28 - DNA, 1714 bp, Homo sapiens
agtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaaggaaaaaa

SEQ ID NO: 29 - DNA, 21 bp, Homo sapiens
  modified_base 20, 21
cuuuggugugggucguggatt

SEQ ID NO: 30 - DNA, 21 bp, Homo sapiens
  modified_base 20, 21: t at positions 20 and 21 are dt
uccacgacccacaccaaagtt

SEQ ID NO: 31 - DNA, 1702 bp, Mus musculus
tctagctctcatcatcctaccgtcatgtctaagtccgagtctcccaaggagccagaacagctgcggaagctcttcatcggagggctgagcttcgaaacaaccgacgagagtctgaggagccattttgagcaatggggaacactaacagactgtgtggtaatgagagatccaaacaccaagagatccaggggctttgggtttgtcacatatgccactgtggaagaagtggatgctgccatgaatgcaagaccacacaaggtggatggaagagttgtggaacctaagagagctgtctcaagagaagattctcagcgaccaggtgcccacttaactgtgaaaaagatctttgttggtggtattaaagaagacactgaagaacatcacctacgagattattttgagcagtatgggaagattgaagtgatagaaattatgactgacagaggcagtgggaaaaagaggggctttgcttttgttacctttgatgaccatgactctgtggataagattgttattcagaaataccatactgtgaatggccacaactgtgaagtaagaaaggctctgtcgaagcaagagatggctagtgcttcatccagtcagagaggtcgcagtggttctggaaactttggtggtggtcgtggaggcggttttggtggcaatgacaattttggtcgaggagggaacttcagtggtcgtggtggctttggtggcagccgtggtggtggtggatatggtggcagtggggatggctataatggatttggcaatgatggaagcaattttggaggtggtggaagctacaatgattttggcaattacaacaatcagtcttccaattttgggccgatgaagggaggaaactttggaggcaggagctctggcccttatggtggtggaggccagtactttgctaaaccacggaaccaaggtggctatggcggttccagcagcagcagtagctatggcagtggcaggaggttctaattacatacagccaggaaacaaagcttagcaggagaggagagccagagaagtgacagggaagctacaggttacaacagatttgtgaactcagccaagcacagtggtggcagggcctagctgctacaaagaagacatgttttagacaatactcatgtgtgtgggcaaaaactccaggactgtatttgtgactaattgtataacaggttattttagtttctgttctgtggaaagtgtaaagcattccaacaaaaggttttactgtagacctttttcacccatgctgttgattgctaaatgtaatagtctgatcatgacgctgaataaatgtgtcttttttttttttttttttaaatgtgctgtgtaaagttagtctattctgaagccatcttggtaaacttccccaacagtgtgaagttagaattccttcagggtggtgccaagttccatttggaatttatttatggttgcttgggtggagaagccattgtcttcaaaaaccttgatgtcgttaaactgccagttactattgtaacttttaatgagtttcaccattgaaagggtcatccaagcaaggtcacaatttggttataaaatggttgttggcacaccctatgcaatatcaaaattgaataacggtatcagataaaataacagatgggaatgaagcttatgtatccattatcatgtgtactcaataaacgatttaattctcttg

SEQ ID NO: 32 - DNA, 1925 bp, Homo sapiens
gagagggcgaaggtaggctggcagatacgttcgtcagcttgctcctttctgcccgtggacgccgccgaagaagcatcgttaaagtctctcttcaccctgccgtcatgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctctaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggcaatgatggtggttatggaggaggcggccctggttactctggaggaagcagaggctatggaagtggtggacagggttatggaaaccagggcagtggctatggcgggagtggcagctatgacagctataacaacggaggcggaggcggctttggcggtggtagtggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattaggaaacaaagcttagcaggagaggagagccagagaagtgacagggaagctacaggttacaacagatttgtgaactcagccaagcacagtggtggcagggcctagctgctacaaagaagacatgttttagacaaatactcatgtgtatgggcaaaaaactcgaggactgtatttgtgactaattgtataacaggttattttagtttctgttctgtggaaagtgtaaagcattccaacaaagggttttaatgtagatttttttttttgcaccccatgctgttgattgctaaatgtaacagtctgatcgtgacgctgaataaatgtcttttttttaatgtgctgtgtaaagttagtctactcttaagccatcttggtaaatttccccaacagtgtgaagttagaattccttcagggtgatgccaggttctatttggaatttatatacaacctgcttgggtggagaagccattgtcttcggaaaccttggtgtagttgaactgatagttactgttgtgacctgaagttcaccattaaaagggattacccaagcaaaatcatggaatggttataaaagtgattgttggcacatcctatgcaatatatctaaattgaataatggtaccagataaaattatagatgggaatgaagcttgtgtatccattatcatgtgtaatcaataaacgatttaattctcttgaa

SEQ ID NO: 33 - DNA, 1780 bp, Homo sapiens
agtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagaaaactttagaaactgttcctttggagaggaaaaagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaaggaaaaaaaaaaaaaaaaaaaaaaaaaaaaaaaaaaaa

SEQ ID NO: 34 - protein, 372 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRRF

SEQ ID NO: 35 - protein, 353 aa, Homo sapiens
MEKTLETVPLERKKREKEQFRKLFIGGLSFETTEESLRNYYEQWGKLTDCVVMRDPASKRSRGFGFVTFSSMAEVDAAMAARPHSIDGRVVEPKRAVAREESGKPGAHVTVKKLFVGGIKEDTEEHHLRDYFEEYGKIDTIEIITDRQSGKKRGFGFVTFDDHDPVDKIVLQKYHTINGHNAEVRKALSRQEMQEVQSSRSGRGGNFGFGDSRGGGGNFGPGPGSNFRGGSDGYGSGRGFGDGYNGYGGGPGGGNFGGSPGYGGGRGGYGGGGPGYGNQGGGYGGGYDNYGGGNYGSGNYNDFGNYNQQPSNYGPMKSGNFGGSRNMGGPYGGGNYGPGGSGGSGGYGGRSRY

SEQ ID NO: 36 - protein, 10 aa, Homo sapiens
ASASSSQRGR

SEQ ID NO: 37 - protein, 14 aa, Homo sapiens
KEDTEEHHLRDYFE